SEQUENCE LISTING (32 sequences)

SEQ ID NO: 1 - DNA, 609 bp, synthetic construct
  misc_feature 1..609: note = Synthetic Polynucleotide
  source 1..609: mol_type = other DNA
atgaaggtcttggcggcaggagttgtgcccctgctgttggttctgcactggaaacatggggcggggagccccctccccatcacccctgtcaacgccacctgtgccatacgccacccatgtcacaacaacctcatgaaccagatcaggagccaactggcacagctcaatggcagtgccaatgccctctttattctctattacacagcccagggggagccgttccccaacaacctggacaagctatgtggccccaacgtgacggacttcccgcccttccacgccaacggcacggagaaggccaagctggtggagctgtaccgcatagtcgtgtaccttggcacctccctgggcaacatcacccgggaccagaagatcctcaaccccagtgccctcagcctccacagcaagctcaacgccaccgccgacatcctgcgaggcctccttagcaacgtgctgtgccgcctgtgcagcaagtaccacgtgggccatgtggacgtgacctacggccctgacacctcgggtaaggatgtcttccagaagaagaagctgggctgtcaactcctggggaagtataagcagatcatcgccgtgttggcccaggccttctag

SEQ ID NO: 2 - protein, 202 aa, synthetic construct
  REGION 1..202: note = Synthetic Polypeptide
MKVLAAGVVPLLLVLHWKHGAGSPLPITPVNATCAIRHPCHNNLMNQIRSQLAQLNGSANALFILYYTAQGEPFPNNLDKLCGPNVTDFPPFHANGTEKAKLVELYRIVVYLGTSLGNITRDQKILNPSALSLHSKLNATADILRGLLSNVLCRLCSKYHVGHVDVTYGPDTSGKDVFQKKKLGCQLLGKYKQIIAVLAQAF

SEQ ID NO: 3 - DNA, 1143 bp, synthetic construct
  misc_feature 1..1143: note = Synthetic Polynucleotide
  source 1..1143: mol_type = other DNA
atgaaagtgctggctgccggcgtggttcctctgctgctggtacttcactggaagcacggtgccggctcaccactccccatcaccccagtaaacgcgacctgcgctattagacacccatgtcacaacaacctgatgaaccagattcgctcccaactggcccagctgaacgggagtgctaatgctctgttcatcttgtattacacagcccaaggagaaccctttcccaataaccttgataaactgtgcggccccaatgtgaccgatttcccccccttccatgctaacgggacggagaaagctaaactggtcgagctgtaccggatagttgtgtacttgggcacatcactcggcaacattacacgggatcagaagatcctgaacccttctgccctttctttgcatagcaagctgaacgccacggctgacatactgaggggtctgctttctaatgtgttgtgccggctttgctctaagtaccatgtaggccatgtagatgtgacgtacggtccggatacttcagggaaagacgtattccagaaaaagaagttggggtgccagctgctggggaaatataagcagatcatagccgttctggcccaggcttttaccagatctcgcaagaagcgcagcaccagatctcgcaagaagcgcagcggggcccccatgatatctctgatcgctgctcttgctgtggactacgtgattggtatggagaatgctatgccctggaatctgcctgcagacctggcatggtttaagaggaacacccttaataaacctgtaatcatgggacgacatacatgggagagtatcggcaggccattgcccgggaggaagaatataattctgagttcccagccttctactgatgatagggtaacttgggtcaagagcgtcgacgaggccatcgccgcttgcggggatgtgcctgaaatcatggttatcgggggcggacgggtgattgagcaatttctgcccaaggcacagaagctgtatctgacccatatagacgccgaggtggagggagatacacactttcccgactatgagcccgatgattgggagagtgtctttagcgaatttcatgacgccgacgcacagaattcccatagctactgcttcgaaattctcgaacggcgctag

SEQ ID NO: 4 - protein, 380 aa, synthetic construct
  REGION 1..380: note = Synthetic Polypeptide
MKVLAAGVVPLLLVLHWKHGAGSPLPITPVNATCAIRHPCHNNLMNQIRSQLAQLNGSANALFILYYTAQGEPFPNNLDKLCGPNVTDFPPFHANGTEKAKLVELYRIVVYLGTSLGNITRDQKILNPSALSLHSKLNATADILRGLLSNVLCRLCSKYHVGHVDVTYGPDTSGKDVFQKKKLGCQLLGKYKQIIAVLAQAFTRSRKKRSTRSRKKRSGAPMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 5 - protein, 414 aa, synthetic construct
  REGION 1..414: note = Synthetic Polypeptide
MESSAKMESGGAGQQPQPQPQQPFLPPAACFFATAAAAAAAAAAAAAQSAQQQQQQQQQQQQAPQLRPAADGQPSGGGHKSAPKQVKRQRSSSPELMRCKRRLNFSGFGYSLPQQQPAAVARRNERERNRVKLVNLGFATLREHVPNGAANKKMSKVETLRSAVEYIRALQQLLDEHDAVSAAFQAGVLSPTISPNYSNDLNSMAGSPVSSYSSDEGSYDPLSPEEQELLDFTNWFTRSRKKRSTRSRKKRSGAPMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 6 - DNA, 3540 bp, synthetic construct
  misc_feature 1..3540: note = Synthetic Polynucleotide
  source 1..3540: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgaagatctaggcaacgcgtctcgaggcggccgccgccaccatggagtcttctgctaaaatggaaagcggaggagcaggtcagcagccgcagccacagccccaacagccgtttctccctccggccgcatgctttttcgcaacagcagccgcagctgcggccgctgccgctgctgctgccgctcaatcagcccaacagcagcaacaacaacagcaacagcagcaacaggccccacaactgagaccagcagctgacgggcaaccctcagggggtggacataagagtgccccaaagcaagttaagcgccaacgctcaagttccccggagctgatgcggtgtaagagacgcctgaactttagcgggttcggctacagcctgccccaacagcaaccagcagccgtagccaggagaaatgaaagggagcgcaatcgggttaagctcgtcaacctcggcttcgcaactctgcgcgaacacgtcccaaatggagcggctaacaagaaaatgagtaaagttgagactctccgcagtgctgtcgaatatattcgggcgcttcaacaacttctcgatgagcacgatgcagtgtctgccgcgtttcaggccggggtcttgtctccgaccatttccccgaactactccaatgacctgaactctatggcaggttctcccgtctcttcctattcttctgatgaaggctcatacgatcctctgagtcctgaggaacaggaattgttggatttcacgaactggttcaccagatctcgcaagaagcgcagcaccagatctcgcaagaagcgcagcggggcccccatgatatctctgatcgctgctcttgctgtggactacgtgattggtatggagaatgctatgccctggaatctgcctgcagacctggcatggtttaagaggaacacccttaataaacctgtaatcatgggacgacatacatgggagagtatcggcaggccattgcccgggaggaagaatataattctgagttcccagccttctactgatgatagggtaacttgggtcaagagcgtcgacgaggccatcgccgcttgcggggatgtgcctgaaatcatggttatcgggggcggacgggtgattgagcaatttctgcccaaggcacagaagctgtatctgacccatatagacgccgaggtggagggagatacacactttcccgactatgagcccgatgattgggagagtgtctttagcgaatttcatgacgccgacgcacagaattcccatagctactgcttcgaaattctcgaacggcgctaggcggccgcgcggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgactggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 7 - protein, 378 aa, synthetic construct
  REGION 1..378: note = Synthetic Polypeptide
MAFTEHSPLTPHRRDLCSRSIWLARKIRSDLTALTESYVKHQGLNKNINLDSADGMPVASTDQWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGIPARGSHYIANNKKMTRSRKKRSTRSRKKRSGAPMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 8 - DNA, 3432 bp, synthetic construct
  misc_feature 1..3432: note = Synthetic Polynucleotide
  source 1..3432: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgaagatctaggcaacgcgtctcgaggcggccgccgccaccatggcctttactgagcatagccccttgacaccgcacagaagggatctttgctccaggtccatctggctcgcacggaagatcaggtcagacctgacagccctgaccgaatcatatgtaaagcaccagggcctgaataaaaacattaacctcgacagcgcagacggaatgccggtggcctcaacagatcagtggtccgaactgacggaggcggagcggttgcaagaaaacttgcaggcgtatcgaacgtttcacgtcctgcttgcccggcttcttgaagaccagcaggttcatttcacgcctacagagggagatttccatcaagcaatccacactttgctcctgcaagtcgcggcctttgcctatcagattgaggaactcatgattcttctcgaatataagattccacggaatgaggcggatgggatgccgattaacgtgggcgacggagggttgtttgagaaaaaactctggggtttgaaagttctgcaagaattgagtcagtggactgtgcgatctatccacgacttgaggttcatctcatcccatcaaacagggatacctgcaagaggctctcattatatagcgaataataaaaaaatgaccagatctcgcaagaagcgcagcaccagatctcgcaagaagcgcagcggggcccccatgatatctctgatcgctgctcttgctgtggactacgtgattggtatggagaatgctatgccctggaatctgcctgcagacctggcatggtttaagaggaacacccttaataaacctgtaatcatgggacgacatacatgggagagtatcggcaggccattgcccgggaggaagaatataattctgagttcccagccttctactgatgatagggtaacttgggtcaagagcgtcgacgaggccatcgccgcttgcggggatgtgcctgaaatcatggttatcgggggcggacgggtgattgagcaatttctgcccaaggcacagaagctgtatctgacccatatagacgccgaggtggagggagatacacactttcccgactatgagcccgatgattgggagagtgtctttagcgaatttcatgacgccgacgcacagaattcccatagctactgcttcgaaattctcgaacggcgctaggcggccgcgcggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgactggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 9 - protein, 510 aa, synthetic construct
  REGION 1..510: note = Synthetic Polypeptide
MISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGAPKKKRKVSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 10 - DNA, 3838 bp, synthetic construct
  misc_feature 1..3838: note = Synthetic Polynucleotide
  source 1..3838: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgaagatctaggcctgcaggcggccgccgccaccatgatctctctgattgccgctctggccgtggactacgtgatcgggatggaaaacgctatgccatggaatctgcccgccgatctggcttggttcaagaggaacaccctgaacaagccagtgatcatgggcagacacacttgggagtccattggccggcccctgcctggacgcaagaacatcattctgagctcccagccctctaccgacgacagggtgacatgggtgaaaagtgtggacgaagccattgccgcttgcggagatgtgcccgagatcatggtcatcggcggagggagagtgatcgagcagttcctgcctaaggcccagaaactgtacctgactcacattgacgctgaggtggaaggggacacccattttcctgattatgagccagacgattgggaaagcgtgttctccgagtttcacgacgccgatgctcagaattctcatagttattgctttgagatcctggaaaggagaggcgcgcctaagaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaggtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 11 - protein, 356 aa, synthetic construct
  REGION 1..356: note = Synthetic Polypeptide
MVSVPTTWCSVALALLVALHEGKGQAAATLEQPASSSHAQGTHLRLRRCSCSSWLDKECVYFCHLDIIWVNTPEQTAPYGLGNPPRRRRRSLPRRCQCSSARDPACATFCLRRPWTEAGAVPSRKSPADVFQTGKTGATTGELLQRLRDISTVKSLFAKRQQEAMREPRSTHSRWRKRTRSRKKRSTRSRKKRSGAPMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 12 - DNA, 3366 bp, synthetic construct
  misc_feature 1..3366: note = Synthetic Polynucleotide
  source 1..3366: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgaagatctaggcaacgcgtctcgaggcggccgccgccaccatggtgagcgttcctacgacatggtgctcagtagctcttgcgcttctggtagcgctccatgaaggcaaaggccaagcggccgcaaccctcgagcagccggcttcatcctcccacgcccaaggcacgcacctcagactcagaaggtgttcctgttcctcatggctggataaagaatgtgtttatttttgccatttggatattatttgggtcaatacgccagaacaaacggccccttacgggcttggtaatccgccacgacggagaaggaggagtctccctcgcagatgccaatgctcatctgcacgcgaccctgcatgcgctacgttctgcctcagacgaccgtggactgaagctggtgcagtgccaagccgcaaatcccctgccgatgtgttccaaacggggaagaccggggctaccactggagaacttctgcaacgcttgcgggacatttctacagtcaaatccctgtttgcaaaaaggcaacaggaggcgatgcgcgagccccgatctacccatagccggtggcggaaaagaaccagatctcgcaagaagcgcagcaccagatctcgcaagaagcgcagcggggcccccatgatatctctgatcgctgctcttgctgtggactacgtgattggtatggagaatgctatgccctggaatctgcctgcagacctggcatggtttaagaggaacacccttaataaacctgtaatcatgggacgacatacatgggagagtatcggcaggccattgcccgggaggaagaatataattctgagttcccagccttctactgatgatagggtaacttgggtcaagagcgtcgacgaggccatcgccgcttgcggggatgtgcctgaaatcatggttatcgggggcggacgggtgattgagcaatttctgcccaaggcacagaagctgtatctgacccatatagacgccgaggtggagggagatacacactttcccgactatgagcccgatgattgggagagtgtctttagcgaatttcatgacgccgacgcacagaattcccatagctactgcttcgaaattctcgaacggcgctaggcggccgcgcggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgactggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 13 - protein, 386 aa, synthetic construct
  REGION 1..386: note = Synthetic Polypeptide
MKLLPSVVLKLFLAAVLSALVTGESLERLRRGLAAGTSNPDPPTVSTDQLLPLGGGRDRKVRDLQEADLDLLRVTLSSKPQALATPNKEEHGKRKKKGKGLGKKRDPCLRKYKDFCIHGECKYVKELRAPSCICHPGYHGERCHGLSLPVENRLYTYDHTTILAVVAVVLSSVCLLVIVGLLMFRYHRRGGYDVENEEKVKLGMTNSHTRSRKKRSTRSRKKRSGAPMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 14 - DNA, 3456 bp, synthetic construct
  misc_feature 1..3456: note = Synthetic Polynucleotide
  source 1..3456: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgaagatctaggcaacgcgtctcgaggcggccgccgccaccatgaaactcctgccgtctgtagtcctgaaactgtttctggctgctgtgttgagtgctctcgttacgggagagtccttggagaggcttcgacgcggtttggcagcaggtacgtcaaacccagatccccctaccgtgagtacggatcagcttctgccgctcggcggaggaagggaccgcaaggtgcgcgatctgcaggaggcggaccttgacctgcttcgagttacattgtcctctaagcctcaagctctggcgacgccgaacaaagaggagcatggtaagcgaaagaagaaaggcaaagggctgggaaaaaaacgcgacccctgtcttcgcaagtataaggacttctgtattcatggagagtgtaagtatgttaaagagcttcgagctcccagttgcatttgccaccctgggtatcacggggaacgctgtcatggcttgtcattgccagttgaaaatcgcttgtatacgtatgaccataccactatcctcgcagtagtagctgttgtcctttccagcgtttgtttgctggtcatcgtcggcttgctgatgtttcggtaccaccgacggggaggttacgatgtagagaacgaggagaaagtcaagctgggcatgacaaatagccataccagatctcgcaagaagcgcagcaccagatctcgcaagaagcgcagcggggcccccatgatatctctgatcgctgctcttgctgtggactacgtgattggtatggagaatgctatgccctggaatctgcctgcagacctggcatggtttaagaggaacacccttaataaacctgtaatcatgggacgacatacatgggagagtatcggcaggccattgcccgggaggaagaatataattctgagttcccagccttctactgatgatagggtaacttgggtcaagagcgtcgacgaggccatcgccgcttgcggggatgtgcctgaaatcatggttatcgggggcggacgggtgattgagcaatttctgcccaaggcacagaagctgtatctgacccatatagacgccgaggtggagggagatacacactttcccgactatgagcccgatgattgggagagtgtctttagcgaatttcatgacgccgacgcacagaattcccatagctactgcttcgaaattctcgaacggcgctaggcggccgcgcggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgactggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 15 - protein, 783 aa, synthetic construct
  REGION 1..783: note = Synthetic Polypeptide
MMDLELPPPGLPSQQDMDLIDILWRQDIDLGVSREVFDFSQRRKEYELEKQKKLEKERQEQLQKEQEKAFFAQLQLDEETGEFLPIQPAQHIQSETSGSANYSQVAHIPKSDALYFDDCMQLLAQTFPFVDDNEVSSATFQSLVPDIPGHIESPVFIATNQAQSPETSVAQVAPVDLDGMQQDIEQVWEELLSIPELQCLNIENDKLVETTMVPSPEAKLTEVDNYHFYSSIPSMEKEVGNCSPHFLNAFEDSFSSILSTEDPNQLTVNSLNSDATVNTDFGDEFYSAFIAEPSISNSMPSPATLSHSLSELLNGPIDVSDLSLCKAFNQNHPESTAEFNDSDSGISLNTSPSVASPEHSVESSSYGDTLLGLSDSEVEELDSAPGSVKQNGPKTPVHSSGDMVQPLSPSQGQSTHVHDAQCENTPEKELPVSPGHRKTPFTKDKHSSRLEAHLTRDELRAKALHIPFPVEKIINLPVVDFNEMMSKEQFNEAQLALIRDIRRRGKNKVAAQNCRKRKLENIVELEQDLDHLKDEKEKLLKEKGENDKSLHLLKKQLSTLYLEVFSMLRDEDGKPYSPSEYSLQQTRDGNVFLVPKSKKPDVKKNTRSRKKRSTRSRKKRSGAPMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 16 - DNA, 4647 bp, synthetic construct
  misc_feature 1..4647: note = Synthetic Polynucleotide
  source 1..4647: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgaagatctaggcaacgcgtctcgaggcggccgccgccaccatgatggatctcgaacttccccctccaggcctgcctagccagcaagacatggaccttatcgacatcctgtggcgccaggatattgatctcggtgtgtctagggaagtctttgacttttcccaaaggaggaaggaatatgagcttgagaaacaaaagaagcttgaaaaggaacggcaagagcagcttcaaaaggagcaggaaaaagcattctttgcgcaattgcagcttgatgaagaaaccggcgaattccttccaatacaaccagctcaacatatccaatccgagacctcaggttccgcaaattactcccaagtggcacatataccgaaatccgacgcgttgtacttcgacgattgtatgcagctgctcgcacaaacgttccctttcgtggacgataacgaagtgagttctgctactttccagtcacttgttccagacatccccggacatattgaatccccagtgtttatagcgacaaaccaagcccaatcacctgagacgagtgtcgcccaagtagccccagtcgatctggatggtatgcaacaggacattgaacaagtctgggaagagctgcttagcattcctgaactgcagtgcctgaacatcgaaaacgacaaattggtagagacaactatggtaccctcacctgaggccaagttgaccgaggtggacaattatcatttttactcttcaatacctagtatggagaaagaagtcggtaattgttcccctcatttcctgaacgcgttcgaagacagctttagcagcatcttgtccacagaagacccaaatcaattgacggtaaactccttgaactcagacgcgacagtgaataccgattttggtgatgaattttattcagcatttatagccgagccaagcatcagtaattctatgcccagccccgcaactctctcccacagtctttcagaattgctcaacggacccatcgatgtgagtgatctgtccctctgcaaagcgtttaaccaaaaccatccggagagtacggccgagttcaacgacagtgatagtggtatttcactcaatacctcaccctccgtggccagtccggagcacagtgttgaatcaagctcctacggggacacattgctgggcctctcagacagcgaagtcgaagaacttgacagcgcccccggatccgtaaagcaaaatgggccgaaaactccggtgcattcatcaggtgatatggtacagccactttcaccaagtcagggacaaagtacgcacgtccatgacgcgcaatgtgaaaatactcctgagaaagagctcccggtatcaccggggcaccggaagaccccttttactaaggacaaacatagtagtcgcttggaggctcatttgactcgagatgagctccgcgcaaaagcactccatattccatttcccgttgaaaagattattaacctcccggtagtggacttcaacgagatgatgtctaaggagcagtttaatgaggcgcagcttgcactgataagggacatacgacgcagaggtaagaataaggtggctgcccaaaactgcaggaagcgaaagctcgagaacattgtagaacttgagcaggaccttgatcacctgaaagatgagaaggaaaaattgcttaaagagaaaggtgagaatgataaaagcttgcatcttctcaaaaaacagctgagcacattgtatcttgaggtcttcagcatgctcagagatgaagatgggaaaccgtattctccgagcgaatacagtctgcagcagacccgggatggcaatgtgttcctcgtacccaaaagcaaaaaacctgatgttaagaagaataccagatctcgcaagaagcgcagcaccagatctcgcaagaagcgcagcggggcccccatgatatctctgatcgctgctcttgctgtggactacgtgattggtatggagaatgctatgccctggaatctgcctgcagacctggcatggtttaagaggaacacccttaataaacctgtaatcatgggacgacatacatgggagagtatcggcaggccattgcccgggaggaagaatataattctgagttcccagccttctactgatgatagggtaacttgggtcaagagcgtcgacgaggccatcgccgcttgcggggatgtgcctgaaatcatggttatcgggggcggacgggtgattgagcaatttctgcccaaggcacagaagctgtatctgacccatatagacgccgaggtggagggagatacacactttcccgactatgagcccgatgattgggagagtgtctttagcgaatttcatgacgccgacgcacagaattcccatagctactgcttcgaaattctcgaacggcgctaggcggccgcgcggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgactggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 17 - protein, 430 aa, synthetic construct
  REGION 1..430: note = Synthetic Polypeptide
MGVLLTQRTLLSLVLALLFPSMASMAAIGSCSKEYRVLLGQLQKQTDLMQDTSRLLDPYIRIQGLDVPKLREHCRERPGAFPSEETLRGLGRRGFLQTLNATLGCVLHRLADLEQRLPKAQDLERSGLNIEDLEKLQMARPNILGLRNNIYCMAQLLDNSDTAEPTKAGRGASQPPTPTPASDAFQRKLEGCRFLHGYHRFMHSVGRVFSKWGESPNRSRRHSPHQALRKGVRRTRPSRKGKRLMTRGQLPRTRSRKKRSTRSRKKRSGAPMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 18 - DNA, 3588 bp, synthetic construct
  misc_feature 1..3588: note = Synthetic Polynucleotide
  source 1..3588: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgaagatctaggcaacgcgtctcgaggcggccgccgccaccatgggggtgcttttgacacagcgcacactcctgagcttggttctggctctcctgttcccgtcaatggcttctatggcggccataggctcatgttctaaagaataccgagtgcttctgggccaacttcagaagcagacggacctcatgcaggatacaagccgccttttggacccatatataaggatccaagggttggatgtgccgaagctgagggaacactgtcgagaacgacctggagcttttccaagcgaagaaaccctcagaggtcttgggaggaggggctttttgcagacgctcaacgcgaccctggggtgtgtgctccaccgacttgcagatctcgagcagcgcctgccaaaagcacaggatctggagcggagtggacttaacatcgaagacctggaaaaattgcagatggctcgaccgaacattcttggccttcgaaacaacatctactgtatggcacagctgctggataactcagatacagccgaacctacaaaagcagggcggggcgcgtcccaaccacctactcccacaccagcctccgatgcctttcagagaaagcttgaagggtgtaggtttctccacggctatcatcgcttcatgcattcagtagggagggtgttctcaaaatggggagagtcacctaatcgatcaaggaggcactcaccccatcaggctcttcggaagggcgttcggcgcactcgcccgagcaggaagggtaaaaggctgatgactcggggtcaactgccaaggaccagatctcgcaagaagcgcagcaccagatctcgcaagaagcgcagcggggcccccatgatatctctgatcgctgctcttgctgtggactacgtgattggtatggagaatgctatgccctggaatctgcctgcagacctggcatggtttaagaggaacacccttaataaacctgtaatcatgggacgacatacatgggagagtatcggcaggccattgcccgggaggaagaatataattctgagttcccagccttctactgatgatagggtaacttgggtcaagagcgtcgacgaggccatcgccgcttgcggggatgtgcctgaaatcatggttatcgggggcggacgggtgattgagcaatttctgcccaaggcacagaagctgtatctgacccatatagacgccgaggtggagggagatacacactttcccgactatgagcccgatgattgggagagtgtctttagcgaatttcatgacgccgacgcacagaattcccatagctactgcttcgaaattctcgaacggcgctaggcggccgcgcggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgactggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 19 - protein, 415 aa, synthetic construct
  REGION 1..415: note = Synthetic Polypeptide
MGVLLTQRTLLSLVLALLFPSMASMAAIGSCSKEYRVLLGQLQKQTDLMQDTSRLLDPYIRIQGLDVPKLREHCRERPGAFPSEETLRGLGRRGFLQTLNATLGCVLHRLADLEQRLGGGNIEDLEKLQMARPNILGLRNNIYCMAQLLDNSDTAEPTKAGRGASQPPTPTPASDAFQRKLEGCRFLHGYHRFMHSVGRVFSKWGESPNRSRRHSPHQALRKGVRRTRPSRKGKRLMTRSRKKRSTRSRKKRSGAPMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 20 - DNA, 3543 bp, synthetic construct
  misc_feature 1..3543: note = Synthetic Polynucleotide
  source 1..3543: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgaagatctaggcaacgcgtctcgaggcggccgccgccaccatgggtgtgttgctcactcaaaggaccttgttgtccctggtcctggcactcttgttcccatctatggcgagtatggcggctattggctcatgctctaaagagtacagagttctgctcggtcaactgcaaaagcaaacggacctgatgcaagatacgtccaggttgctcgacccttatattaggatccagggactcgacgttccgaagcttagagagcactgtcgcgagcgaccaggtgcctttcccagtgaagaaacactgaggggtttgggcaggcgcggtttcttgcagacccttaatgctacattggggtgcgtattgcatcgcctggcagatctcgagcaaagactcggaggtggcaatatcgaggatcttgaaaagctccagatggctcgcccgaacatactcgggctgaggaataatatatattgtatggctcaactcctcgacaactcagacactgcggagcctacgaaagcaggaaggggcgcatcacaaccacctacacctactcctgccagtgatgcctttcagcgcaagcttgagggctgccgatttctccacggctatcatcggttcatgcactccgttggccgagtgttttcaaaatggggggaatctcctaaccgatctagaaggcactcacctcaccaggcactgcggaaaggcgttaggagaacccggccaagccgcaaaggaaagcgcctgatgaccagatctcgcaagaagcgcagcaccagatctcgcaagaagcgcagcggggcccccatgatatctctgatcgctgctcttgctgtggactacgtgattggtatggagaatgctatgccctggaatctgcctgcagacctggcatggtttaagaggaacacccttaataaacctgtaatcatgggacgacatacatgggagagtatcggcaggccattgcccgggaggaagaatataattctgagttcccagccttctactgatgatagggtaacttgggtcaagagcgtcgacgaggccatcgccgcttgcggggatgtgcctgaaatcatggttatcgggggcggacgggtgattgagcaatttctgcccaaggcacagaagctgtatctgacccatatagacgccgaggtggagggagatacacactttcccgactatgagcccgatgattgggagagtgtctttagcgaatttcatgacgccgacgcacagaattcccatagctactgcttcgaaattctcgaacggcgctaggcggccgcgcggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgactggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 21 - protein, 948 aa, synthetic construct
  REGION 1..948: note = Synthetic Polypeptide
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPTTCSNTIDLPMSPRTLDSLMQFGNNGEGAEPSAGGQFESLTFDMELTSECATSPMTRSRKKRSTRSRKKRSGAPMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 22 - DNA, 5142 bp, synthetic construct
  misc_feature 1..5142: note = Synthetic Polynucleotide
  source 1..5142: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgaagatctaggcaacgcgtctcgaggcggccgccgccaccatggctcaatggaaccaattgcaacaacttgatactcgatacctggagcaactccatcagctgtactcagattcatttccgatggagctccggcagtttctggctccatggatagaatcacaggattgggcctacgcggcttcaaaggaaagtcacgcgaccctcgtatttcacaacctcctgggggaaattgatcagcaatatagtcggtttctgcaagaaagcaacgtcttgtaccaacataacttgaggaggattaagcaatttctccaaagccggtacttggagaagcctatggaaatcgctcgcattgtagcgaggtgcctgtgggaggagagtcgccttctgcagaccgccgccacagccgctcaacaaggtgggcaggctaatcatcccaccgctgctgtggtcaccgaaaagcagcagatgctcgagcaacaccttcaggacgtcagaaagagggttcaggatctggaacaaaagatgaaagtcgtggagaatttgcaggatgattttgattttaactacaagaccttgaaatcccaaggcgatatgcaagatcttaatggaaacaaccagtcagtgaccaggcagaagatgcaacaacttgaacaaatgttgactgccctcgatcaaatgagacgcagtatagtaagcgagttggccggtcttctctcagcgatggagtacgtacagaaaacgctgactgatgaagagctggctgactggaagcggcggcagcagatagcttgcatcggtgggcccccaaatatatgcctggatcgcctggagaactggataacgagtctggccgagagtcagctgcagactcgccagcagatcaaaaaactggaagaactccagcaaaaagtttcatacaagggagatccgattgtacagcatcgccccatgctggaagagcgaattgttgaactctttagaaatttgatgaagagcgcgttcgttgtagagcggcagccgtgtatgcctatgcacccggaccggccacttgtgataaagaccggagttcagtttacaacaaaggtccgactgcttgtcaaatttccggaattgaactatcaattgaagatcaaggtttgcatcgataaggattctggcgatgttgcggcgttgcgcggcagccgcaagtttaacatacttggcactaataccaaggttatgaacatggaggagtccaacaacggctcactcagtgctgaattcaagcaccttacgctgagagaacagagatgcggtaacgggggacgcgccaactgtgatgcctcacttattgtcactgaagagcttcacctgataacgttcgaaactgaggtttaccatcaaggcctgaaaatagatctggagactcattctctgcccgttgtcgttatttctaacatttgccagatgcccaatgcatgggcgagcattctgtggtataatatgctgacaaacaatccgaagaacgtcaacttcttcacgaagccgccgatcggtacctgggatcaggtggcggaagtccttagctggcagttcagctccaccaccaagaggggtttgtcaatagagcaattgactacgttggctgagaagctcctgggacctggagtaaattacagtgggtgtcagatcacatgggctaaattttgcaaggaaaacatggcaggtaagggtttttccttttgggtctggcttgataacataatagatctcgtcaaaaagtacatactcgcgctgtggaacgagggttacataatgggttttatatctaaagaaagggagagagcgattttgtccactaaaccaccgggcacgtttcttctccgattttccgagtcaagcaaagaaggtggcgtaacatttacctgggtcgaaaaggacattagtggaaagacgcaaattcaaagtgtggaaccttatacgaaacagcaactgaataacatgtcctttgcggagataataatggggtacaaaattatggatgctactaatatactcgttagccctctggtctacctttacccagacatccccaaggaggaggcgttcggaaagtattgccgaccggaatcccaagagcatcccgaagcggaccccggtagtgccgcaccctaccttaaaactaagttcatttgcgtcaccccaacgacgtgcagcaacacaatagacttgccgatgagtccccgaacgctcgatagtctcatgcagttcggaaacaatggcgagggagctgagccctccgccggcggacaatttgagagcctcacctttgacatggagcttacatcagagtgcgcgacaagcccgatgaccagatctcgcaagaagcgcagcaccagatctcgcaagaagcgcagcggggcccccatgatatctctgatcgctgctcttgctgtggactacgtgattggtatggagaatgctatgccctggaatctgcctgcagacctggcatggtttaagaggaacacccttaataaacctgtaatcatgggacgacatacatgggagagtatcggcaggccattgcccgggaggaagaatataattctgagttcccagccttctactgatgatagggtaacttgggtcaagagcgtcgacgaggccatcgccgcttgcggggatgtgcctgaaatcatggttatcgggggcggacgggtgattgagcaatttctgcccaaggcacagaagctgtatctgacccatatagacgccgaggtggagggagatacacactttcccgactatgagcccgatgattgggagagtgtctttagcgaatttcatgacgccgacgcacagaattcccatagctactgcttcgaaattctcgaacggcgctaggcggccgcgcggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgactggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 23 - protein, 948 aa, synthetic construct
  REGION 1..948: note = Synthetic Polypeptide
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPFLKTKFICVTPTTCSNTIDLPMSPRTLDSLMQFGNNGEGAEPSAGGQFESLTFDMELTSECATSPMTRSRKKRSTRSRKKRSGAPMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 24 - DNA, 5142 bp, synthetic construct
  misc_feature 1..5142: note = Synthetic Polynucleotide
  source 1..5142: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgaagatctaggcaacgcgtctcgaggcggccgccgccaccatggcccagtggaatcaacttcaacaacttgatacccgctacctggaacaacttcatcaactctacagtgatagctttcctatggagctcagacagttcctcgcgccgtggattgaaagccaggactgggcgtatgctgcttcaaaggagagtcatgcaactctcgtattccataatctgctcggagagatcgatcaacaatacagcaggtttctgcaggaatctaacgtgctttatcaacacaatctgaggcggattaaacaattcctgcaatctcgatatcttgaaaaacccatggagattgcccggatcgtcgcacgctgtctgtgggaagagagtcgactgttgcagacggcagctactgctgcccagcagggcggacaggcgaatcatccgactgccgctgtagtgacggaaaaacaacaaatgctggagcaacatctgcaagacgtcagaaaacgagtacaggacctcgaacagaagatgaaagtggtcgaaaacttgcaagacgacttcgattttaattataaaacgctgaaatcccagggggatatgcaagaccttaacggcaataatcagtctgttaccagacagaaaatgcagcagctcgagcaaatgctgacggctcttgaccagatgagaagaagcatagtctcagaattggcaggcctcctctctgccatggagtacgtacagaagacgttgacagatgaggaactcgctgattggaagagacgccaacagattgcttgcatagggggtccacctaatatatgcctcgaccggttggaaaactggataacatctttggctgaatctcagctccaaacacgccagcaaataaaaaagcttgaggagttgcagcagaaggttagttacaagggagatcccattgtgcagcataggccaatgctggaagagaggattgttgagctgtttaggaatctgatgaaaagtgccttcgtagtggagagacagccatgcatgccaatgcatcctgacagaccactggttataaagactggagttcaatttacaacaaaagttagactcctggtcaagttcccggagctcaactatcaactgaagataaaagtctgtattgacaaggatagtggcgatgttgcagccttgaggggtagccgaaaatttaacattctcggaacgaacacaaaagtgatgaacatggaggaatcaaataatggttctctctccgcagagtttaagcaccttactctgcgcgaacaacgctgcggaaacggcggccgggccaattgcgatgccagccttatagtgacggaagagctgcatctgataactttcgagacggaagtctatcatcaaggcctcaaaattgacttggagactcactccttgcccgtagtggttatttcaaatatatgccaaatgcctaatgcatgggcatcaatcctttggtacaatatgcttactaataacccaaagaacgttaatttcttcactaaacctccaattggtacttgggaccaagttgccgaggtattgagttggcagttttcctcaacaactaagagagggttgagcatagaacagctgaccacactggcggagaaacttctgggacccggcgtcaactattctggatgtcagattacctgggccaaattctgcaaagaaaatatggctggcaagggtttcagcttctgggtatggcttgacaatataattgacctcgtaaaaaagtacatcctcgctctttggaatgaaggttatattatgggctttataagtaaagagcgagagcgggctatcttgtcaacgaagcccccgggtacgttcctgttgcgattctcagagagctctaaggagggtggcgtgactttcacgtgggtagagaaagacatctctggtaagactcaaatccaatcagttgaaccgtacacaaagcaacaactgaataacatgtccttcgcagaaattatcatggggtacaagattatggacgcgaccaacatcctggtcagtccgctcgtttatctctatcccgatatacctaaagaagaagcatttggaaagtactgccggccagaatcacaagagcatcctgaggctgatcctggttccgctgcaccgttcttgaaaacgaaattcatatgtgtcactcctacaacctgttcaaacacgatagaccttccaatgtctcctaggacgttggacagccttatgcagtttggtaataatggggagggggccgagccgtccgcggggggtcagtttgagagccttacattcgatatggagctgacctctgagtgtgcaacttctccgatgaccagatctcgcaagaagcgcagcaccagatctcgcaagaagcgcagcggggcccccatgatatctctgatcgctgctcttgctgtggactacgtgattggtatggagaatgctatgccctggaatctgcctgcagacctggcatggtttaagaggaacacccttaataaacctgtaatcatgggacgacatacatgggagagtatcggcaggccattgcccgggaggaagaatataattctgagttcccagccttctactgatgatagggtaacttgggtcaagagcgtcgacgaggccatcgccgcttgcggggatgtgcctgaaatcatggttatcgggggcggacgggtgattgagcaatttctgcccaaggcacagaagctgtatctgacccatatagacgccgaggtggagggagatacacactttcccgactatgagcccgatgattgggagagtgtctttagcgaatttcatgacgccgacgcacagaattcccatagctactgcttcgaaattctcgaacggcgctaggcggccgcgcggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgactggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 25 - protein, 948 aa, synthetic construct
  REGION 1..948: note = Synthetic Polypeptide
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPTTCSNTIDLPMEPRTLDSLMQFGNNGEGAEPSAGGQFESLTFDMELTSECATSPMTRSRKKRSTRSRKKRSGAPMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 26 - DNA, 5142 bp, synthetic construct
  misc_feature 1..5142: note = Synthetic Polynucleotide
  source 1..5142: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgaagatctaggcaacgcgtctcgaggcggccgccgccaccatggctcaatggaaccagctccaacagctcgacaccaggtacctggagcaattgcatcaactctatagtgatagcttccccatggaactgagacaattccttgcaccatggatagagtcacaggattgggcgtacgctgcaagcaaagaatctcatgcaaccctcgtatttcataatctcctcggtgagatcgaccagcaatactcacggttccttcaggagtctaatgtgctttatcaacacaaccttcgccggattaagcagttcttgcagagccgatatctcgagaagcctatggaaatagctaggatcgttgcaaggtgcttgtgggaagaaagtagacttctccaaactgcagcaacggctgcgcagcagggtggccaggccaaccacccgacagccgccgtggtaacggaaaaacagcagatgcttgaacaacaccttcaggacgtccgcaaaagagtacaggacctggaacaaaaaatgaaggtcgtcgaaaaccttcaggatgacttcgattttaactataaaaccctcaaatctcagggagatatgcaagacctcaacggtaataatcagagcgtcactcgacagaagatgcagcaacttgagcaaatgcttacggcactggatcagatgcgaaggtccattgtctccgaacttgccgggctcctgtcagccatggagtatgtacagaaaactttgaccgacgaggagttggcagactggaagaggaggcaacagatagcctgcattggcgggccccctaacatttgccttgacaggttggaaaactggattacatctctggcggagagtcaactgcagacacggcagcaaataaaaaaactggaagagctccagcagaaggtgagttacaagggggacccaattgtgcagcatcgcccaatgctggaagaacggattgttgaattgtttaggaacctcatgaaatccgcgtttgtcgtagagagacagccctgtatgccgatgcatcctgatcgacccctcgtgatcaaaactggtgtacaattcacaacaaaagtgaggttgctggtcaagttccctgaactcaactaccaactgaagattaaagtgtgcattgacaaggattctggagacgtggcggcacttcgcggttcaagaaagtttaacatactcggcactaatacaaaagttatgaatatggaggagtccaacaacgggtcactgtccgccgaatttaagcacttgacgcttcgcgagcaacgatgtggcaatgggggcagagcaaattgcgacgcttctttgattgtcactgaggaacttcacctgattaccttcgaaaccgaggtataccaccaagggctgaaaattgacttggaaacccatagtctccccgtggtggtcattagtaacatttgccagatgcctaatgcttgggcaagcatcctttggtacaacatgcttacaaacaatccgaagaacgtcaacttctttaccaagccgcccattggaacatgggatcaagtggcggaagtcctcagttggcagttttccagtaccacgaagcgaggactttctatcgagcaactgacgacgctggcggaaaaactcttgggacctggagttaattactccgggtgtcagataacatgggccaaattctgcaaggagaacatggcggggaaaggtttcagcttttgggtttggttggataacataattgatctcgtaaagaaatacatactggccctctggaatgaaggttatatcatgggcttcattagtaaagaacgggagagggccatacttagcactaagcctccaggaacgtttcttctcagattttctgagtcttcaaaggagggaggcgtgaccttcacctgggttgaaaaagatatctcaggaaagacacagattcagtccgtcgaaccatatacgaaacagcaactcaataacatgagtttcgcagaaataatcatgggatataagattatggacgcaactaatatactggtgagtccactcgtttatctctaccccgatatcccgaaggaggaagcattcggaaaatactgcagacctgaatcccaggaacatccagaggccgacccaggctctgccgctccatatttgaaaactaaatttatctgcgtaacacctacaacttgcagcaatacgatcgatttgcccatggaaccacgcaccctcgatagccttatgcaattcgggaataatggggaaggagcagaaccctctgcaggaggacaatttgagagcctcacattcgatatggagttgacgtcagaatgcgcaaccagtccaatgaccagatctcgcaagaagcgcagcaccagatctcgcaagaagcgcagcggggcccccatgatatctctgatcgctgctcttgctgtggactacgtgattggtatggagaatgctatgccctggaatctgcctgcagacctggcatggtttaagaggaacacccttaataaacctgtaatcatgggacgacatacatgggagagtatcggcaggccattgcccgggaggaagaatataattctgagttcccagccttctactgatgatagggtaacttgggtcaagagcgtcgacgaggccatcgccgcttgcggggatgtgcctgaaatcatggttatcgggggcggacgggtgattgagcaatttctgcccaaggcacagaagctgtatctgacccatatagacgccgaggtggagggagatacacactttcccgactatgagcccgatgattgggagagtgtctttagcgaatttcatgacgccgacgcacagaattcccatagctactgcttcgaaattctcgaacggcgctaggcggccgcgcggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgactggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 27 - protein, 498 aa, synthetic construct
  REGION 1..498: note = Synthetic Polypeptide
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTTRSRKKRSTRSRKKRSGAPMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 28 - DNA, 3792 bp, synthetic construct
  misc_feature 1..3792: note = Synthetic Polynucleotide
  source 1..3792: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgaagatctaggcaacgcgtctcgaggcggccgccgccaccatgcaatcaaaggtgttgctggcggtggccctttggttgtgtgtcgagacccgagcggcctccgtgggtcttccctccgtttctctcgatctccctcggttgagcatccaaaaagacatccttaccataaaagcaaacactaccttgcagatcacatgcagaggtcagagagatctcgattggttgtggcccaataatcagagcgggtccgaacagcgggttgaggtgaccgagtgctcagatggacttttctgtaagaccctcacgatacccaaagttataggcaacgatactggtgcctacaagtgcttctaccgagaaacggatttggcctcagttatatacgtttatgtgcaagattaccgctccccattcatagcgtcagtcagcgaccaacacggcgttgtctacattacggaaaacaagaataagacggtcgtgataccgtgtctcggaagcatctcaaatctgaatgtctccctgtgcgctcgctaccctgagaaaaggtttgttcctgacggtaacagaatatcctgggatagtaaaaaaggttttacgattccctcctatatgattagttatgctggcatggtgttttgcgaggctaaaatcaacgatgagtcctaccagtcaataatgtatattgtggtggttgtgggctacaggatctacgatgtggttttgtctcccagccatggaatcgagttgtctgtaggggaaaaactggtgcttaactgcacagcgagaaccgaactcaacgttggaatagacttcaactgggagtacccgagttccaaacaccaacacaaaaaactcgtaaacagggatctgaagacccagtcagggtctgagatgaaaaaatttctttctactctgacaatagatggagtcacccgctccgatcaagggctctacacttgtgcggcttcttcagggctgatgaccaaaaaaaattcaactaccagatctcgcaagaagcgcagcaccagatctcgcaagaagcgcagcggggcccccatgatatctctgatcgctgctcttgctgtggactacgtgattggtatggagaatgctatgccctggaatctgcctgcagacctggcatggtttaagaggaacacccttaataaacctgtaatcatgggacgacatacatgggagagtatcggcaggccattgcccgggaggaagaatataattctgagttcccagccttctactgatgatagggtaacttgggtcaagagcgtcgacgaggccatcgccgcttgcggggatgtgcctgaaatcatggttatcgggggcggacgggtgattgagcaatttctgcccaaggcacagaagctgtatctgacccatatagacgccgaggtggagggagatacacactttcccgactatgagcccgatgattgggagagtgtctttagcgaatttcatgacgccgacgcacagaattcccatagctactgcttcgaaattctcgaacggcgctaggcggccgcgcggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgactggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 29 - protein, 931 aa, synthetic construct
  REGION 1..931: note = Synthetic Polypeptide
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFITRSRKKRSTRSRKKRSGAPMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 30 - DNA, 5091 bp, synthetic construct
  misc_feature 1..5091: note = Synthetic Polynucleotide
  source 1..5091: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgaagatctaggcaacgcgtctcgaggcggccgccgccaccatgcagtcaaaggtccttctcgcggtagctctgtggttgtgcgtagagacacgggccgcttcagtggggctgccttcagtaagcctcgatttgccccggctttcaatacagaaagatattctcacaatcaaggctaataccactctgcaaattacctgtcgcggccaaagggatctggattggctctggccgaataatcaatcagggagtgaacagcgggtcgaagtgaccgagtgttctgacggacttttttgcaaaacacttactatcccaaaagtaatcgggaacgataccggggcttacaagtgtttctacagagagacagatcttgctagtgtgatttacgtatatgtgcaagattaccgcagccctttcatagcatccgtatcagaccaacatggagttgtatacattacagagaacaagaacaaaactgtagttattccctgtttggggtctataagcaacctcaacgtaagcctctgcgctagatacccagagaaacgattcgtcccggatgggaatagaatttcctgggatagcaagaagggcttcactattccgagctacatgatatcatacgcaggtatggtgttctgcgaagcgaagataaacgatgagagttatcagtcaatcatgtacatagtggtggtcgttgggtacagaatttatgatgttgtcctcagtccgtcccatggcattgaactgagtgtcggggaaaaattggtgctgaattgtaccgctaggacagaattgaacgttggcattgactttaactgggagtatccgagctcaaagcaccagcataaaaagttggttaacagagatctgaaaacccagtctggcagtgaaatgaaaaagttcctctctacgctgacaatagacggtgttacgaggtctgatcagggtttgtacacatgtgctgcatcttccggtttgatgacaaagaaaaactccacgtttgttcgcgtgcatgagaagccgtttgtggcgttcggctcaggaatggagtccctcgttgaagcgacggtaggcgagcgggtccggatccccgctaaatacttgggctacccaccaccggagataaaatggtataagaatggtataccgcttgagagtaatcatacgataaaagcgggtcatgtactcaccataatggaagtaagcgagagagatactggaaattacaccgttatcctgacaaatccaatatccaaagagaagcagtcacacgttgtatcactcgtggtttatgtcccccctcaaataggagaaaagtcactcatcagtccggtggatagttatcaatacgggacgacccagactctcacatgcacagtatatgccatccctccaccccaccatattcattggtattggcagcttgaagaggaatgcgccaatgagccttcacaggcagtgtcagttacaaatccttacccttgtgaggaatggaggagcgtcgaggacttccaggggggtaataagatagaagtcaataagaatcaatttgcactcatagaaggaaagaacaagacggtttctactttggtgatacaagccgcaaatgtgtccgcactctataagtgcgaggcagtcaacaaagttggtcgcggcgagcgggtgataagcttccacgtcacgaggggaccggaaatcactcttcaacccgatatgcaacccacggaacaggaatctgtttcactttggtgtacggcggataggagtacgtttgagaacctcacatggtataaattgggaccgcagcccttgcctatccacgtgggggagcttcccactcctgtttgcaagaatctggatacactctggaaactgaatgctactatgttcagcaattctaccaatgacatactcattatggaattgaaaaatgcgtcactccaagatcagggagattacgtatgcctcgctcaggaccgcaagactaaaaagaggcactgtgttgtccggcagcttacagttcttgaacgcgtggcgcctaccattaccggcaatctggagaatcaaactacgtcaatcggggagagcatcgaggtctcatgcacggccagcggcaatcctccgccccagattatgtggtttaaagacaatgagacgctggtggaggatagtgggattgtacttaaggatgggaatcgcaacctcaccattcgaagagtgcgcaaggaggacgaagggttgtacacctgccaagcatgctccgttctgggatgcgcaaaagttgaagcattctttataaccagatctcgcaagaagcgcagcaccagatctcgcaagaagcgcagcggggcccccatgatatctctgatcgctgctcttgctgtggactacgtgattggtatggagaatgctatgccctggaatctgcctgcagacctggcatggtttaagaggaacacccttaataaacctgtaatcatgggacgacatacatgggagagtatcggcaggccattgcccgggaggaagaatataattctgagttcccagccttctactgatgatagggtaacttgggtcaagagcgtcgacgaggccatcgccgcttgcggggatgtgcctgaaatcatggttatcgggggcggacgggtgattgagcaatttctgcccaaggcacagaagctgtatctgacccatatagacgccgaggtggagggagatacacactttcccgactatgagcccgatgattgggagagtgtctttagcgaatttcatgacgccgacgcacagaattcccatagctactgcttcgaaattctcgaacggcgctaggcggccgcgcggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgactggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 31 - protein, 417 aa, synthetic construct
  REGION 1..417: note = Synthetic Polypeptide
MKVLAAGVVPLLLVLHWKHGAGDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMTRSRKKRSTRSRKKRSGAPMISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 32 - DNA, 3549 bp, synthetic construct
  misc_feature 1..3549: note = Synthetic Polynucleotide
  source 1..3549: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgaagatctaggcaacgcgtctcgaggcggccgccgccaccatgaaagtccttgccgccggagttgtccctctcttgcttgtactccactggaagcacggtgcaggagattatcgctcaccatttatagcgagcgtgtcagatcaacatggcgtggtctatataactgagaataagaataaaacggtcgttatcccctgtttgggtagcatatctaacctcaacgttagtttgtgcgctcgataccctgaaaagcggttcgtacccgatggcaatcggatcagttgggacagtaaaaaaggcttcactattccgtcatacatgatcagctatgcagggatggttttctgcgaagctaaaatcaatgacgagagttatcagagcattatgtacatcgtagttgtcgttgggtatcgaatttacgacgttgtgctgtccccttctcacggaattgaactctccgttggagaaaaactcgtattgaactgtacggcacgcacggaattgaatgtagggatagatttcaactgggagtatccatcatctaaacatcaacataaaaaacttgtaaatcgagacctgaagacacagagtgggtccgaaatgaaaaaatttcttagtactctgactatcgacggggtaacaagatctgatcaagggctgtacacctgtgcggccagctcaggtcttatgacaaaaaaaaactccacttttgtcagagtgcatgaaaagccgttcgtggcatttggttcaggtatgaccagatctcgcaagaagcgcagcaccagatctcgcaagaagcgcagcggggcccccatgatatctctgatcgctgctcttgctgtggactacgtgattggtatggagaatgctatgccctggaatctgcctgcagacctggcatggtttaagaggaacacccttaataaacctgtaatcatgggacgacatacatgggagagtatcggcaggccattgcccgggaggaagaatataattctgagttcccagccttctactgatgatagggtaacttgggtcaagagcgtcgacgaggccatcgccgcttgcggggatgtgcctgaaatcatggttatcgggggcggacgggtgattgagcaatttctgcccaaggcacagaagctgtatctgacccatatagacgccgaggtggagggagatacacactttcccgactatgagcccgatgattgggagagtgtctttagcgaatttcatgacgccgacgcacagaattcccatagctactgcttcgaaattctcgaacggcgctaggcggccgcgcggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgactggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

(19) United States
(12) Patent Application Publication
(10) Pub. No.: US 2025/0195696 A1
(43) Pub. Date: Jun. 19, 2025

(54) RETINAL PROTECTIVE FACTOR 2 (RPF2) PROTEIN DELIVERED BY ADENO-ASSOCIATED VIRUS EXPRESSION

(71) Applicant: University of Florida Research Foundation, Incorporated, Gainesville, FL (US)

(72) Inventors: John Ash, Gainesville, FL (US); Clayton Pio Santiago, Gainesville, FL (US)

(73) Assignee: University of Florida Research Foundation, Incorporated, Gainesville, FL (US)

(21) Appl. No.: 18/981,283

(22) Filed: Dec. 13, 2024

Related U.S. Application Data
(62) Division of application No. 16/977,124, filed on Sep. 1, 2020.
(60) Provisional application No. 62/637,485, filed on Mar. 2, 2018.

(51) Int. Cl. A61K 48/00; A61P 27/02; C07K 14/54; C12N 9/06

(52) U.S. Cl. CPC A61K 48/0075; A61P 27/02; C07K 14/5415; C12N 9/003

(57) ABSTRACT

In some aspects, the disclosure relates to compositions and methods useful for maintaining or improving retinal function and/or morphology. The disclosure is based, in part, on isolated nucleic acids encoding certain neurotrophic factors (e.g., leukemia inhibitory factor (LIF), etc.) and gene therapy vectors (e.g., recombinant adeno-associated virus (rAAV) vectors) encoding the same. In some embodiments, isolated nucleic acids and gene therapy vectors described by the disclosure are useful for treatment of certain diseases or disorders of the eye, for example retinal degeneration, retinitis pigmentosa (RP), age-related macular degeneration (AMD), glaucoma, etc.

### RELATED APPLICATIONS

This application claims the benefit under 35 U.S.C. 119(e) of the filing date of U.S. Provisional Application Ser. No. 62/637,485, filed Mar. 2, 2018, entitled “DRUG STABILIZED NEUROPROTECTIVE FACTOR (RPF2) DELIVERED BY ADENO-ASSOCIATED VIRUS EXPRESSION”, the entire contents of which are incorporated herein by reference.

### FEDERALLY SPONSORED RESEARCH

This invention was made with government support under grant number EY016459 awarded by the National Institutes of Health. The government has certain rights in the invention.

### BACKGROUND

Vision loss is often due to the loss of neurons that either initiate the visual response to light (photoreceptors), or the neurons that transmit the signal to the brain (bipolar cells, ganglion cells, etc.). To date, mutations in approximately 250 genes have been identified to cause inherited retinal diseases. For example, retinitis pigmentosa (RP) is a monogenetic disease and patients often have impaired night and peripheral vision beginning from childhood, which progressively worsens until central vision is lost. Other retinal degenerative disorders such as age-related macular degeneration (AMD) are caused by a multitude of factors such as polymorphisms in complement factor H, obesity, smoking and hypertension.

It has been observed that neuroprotective cytokines such as leukemia inhibitory factor (LIF) or ciliary neurotrophic factor (CNTF) can protect photoreceptors from a broad range of insults, including mechanical injury and multiple mutations that cause inherited retinal degeneration. However, long-term effects of highly-expressed neurotrophic factors (e.g., LIF, CNTF, etc.) have been shown to be detrimental to retinal function and may promote inflammation.

### SUMMARY

Aspects of the disclosure elate to compositions and methods for delivering one or more transgenes (e.g., therapeutic proteins) to a subject, for example by adeno-associated virus (AAV)-based expression. The disclosure is based, in part, on expression vectors (e.g., AAV expression vectors) comprising a nucleic acid sequence encoding a fusion protein that includes a therapeutic protein and a drug-reactive domain (e.g., a drug-sensitive destabilisation domain (DD), such as a dihydrofolate reductase (DHFR) protein). Without wishing to be bound by any particular theory, expression of therapeutic proteins comprising a DD may be controlled (e.g., induced or inhibited) by altering the amount of an agent, such as a small molecule, that interacts with the DD and prevents degradation of the therapeutic protein. Accordingly, compositions of the disclosure are useful, in some embodiments, for delivering and/or modulating expression of therapeutic transgenes in a cell or subject.

In some aspects, the disclosure provides an isolated nucleic acid that encodes a transgene comprising a fusion protein that include, a therapeutic protein and a destabilization domain (DD). In some embodiments, the therapeutic protein is selected from a neurotrophic factor (e.g., LIF, CNTF, EDN2, OSM, etc.), transcription factor (e.g., ASCL1, NRF2, STAT3, etc.), growth factor (e.g., HBEGF), protective factor, and a growth factor receptor subunit (e.g., VEGFR2, etc.). In some embodiments, the therapeutic protein is a recombinase (e.g., Cre recombinase).

In some embodiments, a fusion protein encodes an Achaete-scute homolog 1 (ASCL1) protein or a fragment thereof. In some embodiments, an ASCL1 protein comprises the amino acid sequence set forth in SEQ ID NO: 5. In some embodiments, the destabilization domain (DD) is a DHFR protein. In some embodiments, a DD (e.g., DHFR protein or a fragment thereof) is capable of being bound by trimethoprim (TMP). In some embodiments, a DHFR protein or fragment thereof is an E. coli DHFR protein, or a fragment thereof. In some embodiments, a nucleic acid sequence encoding a protein is linked to a nucleic acid sequence encoding a DD by a linker sequence. In some embodiments, a linker sequence encodes one or more furin cleavage sites. In some embodiments, an isolated nucleic acid further comprises adeno-associated virus (AAV) inverted terminal repeats that flank the transgene. In some embodiments, an isolated nucleic acid comprises the nucleic acid sequence set forth in SEQ ID NO: 6.

In some embodiments, a fusion protein encodes a Ciliary neurotrophic factor (CNTF) protein or a fragment thereof. In some embodiments, a CNTF protein comprises the amino acid sequence set forth in SEQ ID NO: 7. In some embodiments, the destabilization domain (DD) is a DHFR protein. In some embodiments, a DD (e.g., DHFR protein or a fragment thereof) is capable of being bound by trimethoprim (TMP). In some embodiments, a DHFR protein or fragment thereof is an E. coli DHFR protein, or a fragment thereof. In some embodiments, a nucleic acid sequence encoding a protein is linked to a nucleic acid sequence encoding a DD by a linker sequence. In some embodiments, a linker sequence encodes one or tore furin cleavage sites. In some embodiments, an isolated nucleic acid further comprises adeno-associated virus (AAV) inverted terminal repeats that flank the transgene. In some embodiments, an isolated nucleic acid comprises the nucleic acid sequence set forth in SEQ ID NO: 8.

In some embodiments, a fusion protein encodes a Cre recombinase (Cre) protein or a fragment thereof. In some embodiments, an Cre protein comprises the amino acid sequence set forth in SEQ ID NO: 9. In some embodiments, the destabilization domain (DD) is a DHFR protein. In some embodiments, a DD (e.g., DHFR protein or a fragment thereof) is capable of being bound by trimethoprim (TMP). In some embodiments, a DHFR protein or fragment thereof is an E. coli DHFR protein, or a fragment thereof. In some embodiments, a nucleic acid sequence encoding a protein is linked to a nucleic acid sequence encoding a DD by a linker sequence. In some embodiments, a linker sequence encodes one or more furin cleavage sites. In some embodiments, an isolated nucleic acid further comprises adeno-associated virus (AAV) inverted terminal repeats that flank the transgene. In some embodiments, an isolated nucleic acid comprises the nucleic acid sequence set forth in SEQ ID NO: 10.

In some embodiments, a fusion protein encodes a Endothelin 2 (EDN2) protein or a fragment thereof. In some embodiments, an EDN2 protein comprises the amino acid sequence set forth in SEQ ID NO: 11. In some embodiments, the destabilization domain (DD) is a DHFR protein. In some embodiments, a DD (e.g., DHFR protein or a fragment thereof) is capable of being bound by trimethoprim (TMP). In some embodiments, a DHFR protein or fragment thereof is an E. coli DHFR protein, or a fragment thereof. In some embodiments, a nucleic acid sequence encoding a protein is linked to a nucleic acid sequence encoding a DD by a linker sequence. In some embodiments, a linker sequence encodes one or more furin cleavage sites. In some embodiments, an isolated nucleic acid further comprises adeno-associated virus (AAV) inverted terminal repeats that flank the transgene. In some embodiments, an isolated nucleic acid comprises the nucleic acid sequence set forth in SEQ ID NO: 12.

In some embodiments, a fusion protein encodes a Heparin-binding EGF-like growth factor (HBEGF) protein or a fragment thereof. In some embodiments, an HBEGF protein comprises the amino acid sequence set forth in SEQ ID NO: 13. In some embodiments, the destabilization domain (DD) is a DHFR protein. In some embodiments, a DD (e.g., DHFR protein or a fragment thereof) is capable of being bound by trimethoprim (TMP). In some embodiments, a DHFR protein or fragment thereof is an E. coli DHFR protein, or a fragment thereof. In some embodiments, a nucleic acid sequence encoding a protein is linked to a nucleic acid sequence encoding a DD by a linker sequence. In some embodiments, a linker sequence encodes one or more furin cleavage sites. In some embodiments, an isolated nucleic acid further comprises adeno-associated virus (AAV) inverted terminal repeats that flank the transgene. In some embodiments, an isolated nucleic acid comprises the nucleic acid sequence set forth in SEQ ID NO: 14.

In some embodiments, a fusion protein encodes a Nuclear factor (erythroid-derived 2)-like 2 growth factor (NRF2) protein or a fragment thereof. In some embodiments, an NRF2 protein comprises the amino acid sequence set forth in SEQ ID NO: 15. In some embodiments, the destabilization domain (DD) is a DHFR protein. In some embodiments, a DD (e.g., DHFR protein or a fragment thereof) is capable of being bound by trimethoprim (TMP). In some embodiments, a DHFR protein or fragment thereof is an E. coli DHFR protein, or a fragment thereof. In some embodiments, a nucleic acid sequence encoding a protein is linked to a nucleic acid sequence encoding a DD by a linker sequence. In some embodiments, a linker sequence encodes one or more furin cleavage sites. In some embodiments, an isolated nucleic acid further comprises adeno-associated virus (AAV) inverted terminal repeats that flank the transgene. In some embodiments, an isolated nucleic acid comprises the nucleic acid sequence set forth in SEQ ID NO: 16.

In some embodiments, a fusion protein encodes an Oncostatin M (OSM) protein or a fragment thereof. In some embodiments, an OSM protein comprises the amino acid sequence set forth in SEQ ID NO: 17. In some embodiments, the destabilization domain (DD) is a DHFR protein. In some embodiments, a DD (e.g., DHFR protein or a fragment thereof) is capable of being bound by trimethoprim (TMP). In some embodiments, a DHFR protein or fragment thereof is an E. coli DHFR protein, or a fragment thereof. In some embodiments, a nucleic acid sequence encoding a protein is linked to a nucleic acid sequence encoding a DD by a linker sequence. In some embodiments, a linker sequence encodes one or more furin cleavage sites. In some embodiments, an isolated nucleic acid further comprises adeno-associated virus (AAV) inverted terminal repeats that flank the transgene. In some embodiments, an isolated nucleic acid comprises the nucleic acid sequence set forth in SEQ ID NO: 18.

In some embodiments, a fusion protein encodes an Oncostatin M (OSM) protein variant (e.g., OSM M2) or a fragment thereof. In some embodiments, an OSM M2 protein comprises the amino acid sequence set forth in SEQ ID NO: 19. In some embodiments, the destabilization domain (DD) is a DHFR protein. In some embodiments, a DD (e.g., DHFR protein or a fragment thereof) is capable of being bound by trimethoprim (TMP). In some embodiments, a DHFR protein or fragment thereof is an E. coli DHFR protein, or a fragment thereof. In some embodiments, a nucleic acid sequence encoding a protein is linked to a nucleic acid sequence encoding a DD by a linker sequence. In some embodiments, a linker sequence encodes one or more furin cleavage sites. In some embodiments, an isolated nucleic acid further comprises adeno-associated virus (AAV) inverted terminal repeats that flank the transgene. In some embodiments, an isolated nucleic acid comprises the nucleic acid sequence set forth in SEQ ID NO: 20.

In some embodiments, a fusion protein encodes a Signal transducer and activator of transcription 3 (STAT3) protein or a fragment thereof. In some embodiments, an STAT3 protein comprises the amino acid sequence set forth in SEQ ID NO: 21. In some embodiments, the destabilization domain (DD) is a DHFR protein. In some embodiments, a DD (e.g., DHFR protein or a fragment thereof) is capable of being bound by trimethoprim (TMP). In some embodiments, a DHFR protein or fragment thereof is an E. coli DHFR protein, or a fragment thereof. In some embodiments, a nucleic acid sequence encoding a protein is linked to a nucleic acid sequence encoding a DD by a linker sequence. In some embodiments, a linker sequence encodes one or more furin cleavage sites. In some embodiments, an isolated nucleic acid further comprises adeno-associated virus (AAV) inverted terminal repeats that flank the transgene. In some embodiments, an isolated nucleic acid comprises the nucleic acid sequence set forth in SEQ ID NO: 22.

In some embodiments, a fusion protein encodes a Signal transducer and activator of transcription 3 (STAT3) protein variant (e.g., STAT3 Y705F, STAT3 S727E, etc.) or a fragment thereof. In some embodiments, an STAT3 protein variant comprises the amino acid sequence set forth in SEQ ID NO: 23 or 25. In some embodiments, the destabilization domain (DD) is a DHFR protein. In some embodiments, a DD (e.g., DHFR protein or a fragment thereof) is capable of being bound by trimethoprim (TMP). In some embodiments, a DHFR protein or fragment thereof is an E. coli DHFR protein, or a fragment thereof. In some embodiments, a nucleic acid sequence encoding a protein is linked to a nucleic acid sequence encoding a DD by a linker sequence. In some embodiments, a linker sequence encodes one or more furin cleavage sites. In some embodiments, an isolated nucleic acid further comprises adeno-associated virus (AAV) inverted terminal repeats that flank the transgene. In some embodiments, an isolated nucleic acid comprises the nucleic acid sequence set forth in SEQ ID NO: 24 or 26.

In some embodiments, a fusion protein encodes a vascular endothelial growth factor receptor 2 (VEGFR2) protein or a fragment thereof. In some embodiments, an VEGFR2 protein comprises one or more Ig-like loop regions, for example Ig-like regions 1-3, Ig-like regions 1-7. Ig-like regions 2-3, etc. In some embodiments, an VEGFR2 protein comprises the amino acid sequence set forth in any one of SEQ ID NOs: 27, 29, and 31. In some embodiments, the destabilization domain (DD) is a DHFR protein. In some embodiments, a DD (e.g., DHFR protein or a fragment thereof) is capable of being bound by trimethoprim (TMP). In some embodiments, a DHFR protein or fragment thereof is an E. coli DHFR protein, or a fragment thereof. In some embodiments, a nucleic acid sequence encoding a protein is linked to a nucleic acid sequence encoding a DD by a linker sequence. In some embodiments, a linker sequence encodes one or more furin cleavage sites. In some embodiments, an isolated nucleic acid further comprises adeno-associated virus (AAV) inverted terminal repeats that flank the transgene. In some embodiments, an isolated nucleic acid comprises the nucleic acid sequence set forth in any one of SEQ ID NOs: 28, 30, or 32.

In some aspects, the disclosure provides recombinant adeno-associated virus (rAAV) particles comprising an isolated nucleic acid as described by the disclosure.

In some aspects, the disclosure provides methods of delivering a transgene to a cell or a subject comprising administering an rAAV comprising an isolated nucleic acid as described by the disclosure to a cell or subject.

In some aspects, the disclosure relates to compositions and methods useful for maintaining or improving retinal function and/or morphology. The disclosure is based, in part, on isolated nucleic acids encoding certain neurotrophic factors (e.g., leukemia inhibitory factor (LIF), etc.) and gene therapy vectors (e.g., recombinant adeno-associated virus (rAAV) vectors) encoding the same. In some embodiments, isolated nucleic acids and gene therapy vectors described by the disclosure are useful for treatment of certain diseases or disorders of the eye, for example retinal degeneration, retinitis pigmentosa (RP), age-related macular degeneration (AMD), glaucoma, etc.

Accordingly, in some aspects, the disclosure provides an isolated nucleic acid comprising a sequence that is at least 75% identical to SEQ ID NO: 1 and encodes a protein, wherein the protein binds to a Leukemia Inhibitory Factor Receptor subunit (LIFR) and/or a Glycoprotein 130 (gp130) subunit.

In some embodiments, the nucleic acid sequence encodes a protein that is at least 95% identical to the sequence set forth in SEQ ID NO: 2.

In some embodiments, the isolated nucleic acid comprises one or more adeno-associated virus (AAV) inverted terminal repeat (ITR) sequences. In some embodiments, the one or more AAV ITR sequences are AAV2 ITR sequences.

In some embodiments, an isolated nucleic acid comprises two AAV ITR sequences, wherein the ITR sequences flank a sequence encoding a protein.

In some embodiments, an isolated nucleic acid further comprises a CBA promoter operably linked to the sequence encoding the protein.

In some embodiments, an isolated nucleic acid further comprises a destabilization domain (DD). In some embodiments, a DD comprises a sequence encoding a dihydrofolate reductase (DHFR) protein, or a fragment thereof. In some embodiments, a DD (e.g., DHFR protein or a fragment thereof) is capable of being bound by trimethoprim (TMP). In some embodiments, a DHFR protein or fragment thereof is an E. coli DHFR protein, or a fragment thereof.

In some embodiments, a nucleic acid sequence encoding a protein is linked to a nucleic acid sequence encoding a DD by a linker sequence. In some embodiments, a linker sequence encodes one or more furin cleavage sites.

In some embodiments, an isolated nucleic acid described by the disclosure comprises the sequence set forth in SEQ ID NO: 3 or encodes the amino acid sequence set forth in SEQ ID NO: 4.

In some aspects, the disclosure provides a recombinant adeno-associated virus (rAAV) comprising: an expression cassette encoding a transgene comprising an isolated nucleic acid as described herein; and one or more adeno-associated virus (AAV) capsid proteins.

In some embodiments, one or more AAV capsid proteins is of a serotype selected from AAV2 or a variant thereof, and AAV6 or a variant thereof. In some embodiments, one or more capsid proteins is of an AAV2* serotype. In some embodiments, one or more capsid proteins is of an AAV6* serotype.

In some aspects, the disclosure provides a composition comprising an isolated nucleic acid as described herein or an rAAV as described herein, and a pharmaceutically acceptable excipient.

In some aspects, the disclosure provides a host cell comprising an isolated nucleic acid as described herein or an rAAV as described herein. In some embodiments, a host cell is a bacterial cell, a mammalian cell, or an insect cell. In some embodiments, a mammalian cell is a HEK293 cell. In some embodiments, an insect cell is an Sf9 cell.

In some aspects, the disclosure provides a method of delivering a transgene to the eye of a subject, the method comprising administering an isolated nucleic acid as described herein, or an rAAV as described herein, or a composition as described herein, to a subject.

In some aspects, the disclosure provides a method for treating a disease or disorder of the eye in a subject in need thereof, the method comprising administering an isolated nucleic acid as described herein, or rAAV as described herein, or composition as described herein, to a subject having or suspected of having a disease or disorder of the eye.

In some embodiments, a subject is a mammal. In some embodiments, a subject is a human or a mouse. In some embodiments, a subject has or is suspected of having retinal degeneration, retinitis pigmentosa (RP), age-related macular degeneration (AMD), or glaucoma.

In some embodiments, administration is intravitreal administration, topical administration, etc.

In some embodiments, methods described herein further comprise the step of administering trimethoprim (TMP) to a subject one or more times. In some embodiments, a subject has been administered TMP prior to administration of an isolated nucleic acid, rAAV, or composition. In some embodiments, a subject has been administered TMP after administration of the isolated nucleic acid, rAAV, or composition. In some embodiments, the concentration of TMP in each administration is between about 1 mg/kg and about 50 mg/kg of a subject.

In some embodiments, administration of an isolated nucleic acid as described herein, or rAAV as described herein, or composition as described herein, results in expression of the transgene in the eye of a subject. In some embodiments, expression of a transgene (e.g., a protein as described by the disclosure) does not result in edema and/or loss of function of retinal cells of the subject.

### BRIEF DESCRIPTION OF DRAWINGS

FIG. 1 shows up-regulation of leukemia inhibitory factor (LIF), Oncostatin M (OSM), and Eosinophil lysophospholipase (CLC), which are all gp130 ligands. Other genes assayed included Receptor-like protein 19 (RLP19), ciliary neurotrophic factor (CNTF). Vascular endothelial growth factor (VEGF), Brain-derived neurotrophic factor (BDNF), and Heparin-binding growth factor 1 (FGF1).

FIG. 2 shows injection of LIF delays photoreceptor degeneration in mice with autosomal dominant RP. On the contrary, injection of LIF05, an antagonist of LIF receptor, accelerates degeneration.

FIGS. 3A-3B show LIF injection protects retinal pigment epithelium (RPE) from degeneration caused by sodium iodate (NAIO₃) injection. FIG. 3A is a schematic depicting experimental design for NAIO₃) injection. FIG. 3B shows data indicating that IF injection increases RPE resistance to NAIO₃.

FIGS. 4A-4B show LIF and STAT3 signaling pathways. FIG. 4A shows a schematic depicting signaling pathways associated with LIF/CNTF receptors. FIG. 4B shows microscopy data indicating loss of LIF-induced phosphorylated STAT3 (pSTST3) in gp130 targeted mice.

FIGS. 5A-5B show the role of gp130 and STAT3 in a genetic model of retinal degeneration. FIG. 5A shows loss of gp130 in rod photoreceptors accelerates photoreceptor cell death in a genetic model of retinal degeneration. FIG. 5B shows loss of STAT3 in rod photoreceptors accelerates photoreceptor cell death in a genetic model of retinal degeneration.

FIG. 6 shows AAV6 (3pMut) has improved targeting of cells in the inner nuclear layer. FIG. 6A (top panel) shows fundus images of the retina comparing mCherry expression in AAV6 and AAV6 (3pMut); (bottom panel) immunohistochemistry cross section of retinas injected intravitreally with AAV6 or AAV6 (3pMut) showing mCherry expression and DAPI stained nuclei. FIG. 6B (top panel) shows fundus images of the retina injected with AAV2-CBA-GFP; (bottom panel): immunohistochemistry cross section of the same retina showing GFP expression. ONL=Outer nuclear layer. INL=Inner nuclear layer and GC=Ganglion cell layer.

FIGS. 7A-7F show long term constitutive expression of AAV-hLIF can have adverse effects on retinal morphology and function. FIG. 7A is a schematic depicting an AAV-hLIF expression construct. FIG. 7B is a schematic depicting a dosing schedule for AAV-hLIF and subsequent data analysis. FIG. 7C shows the OCT scoring system rubric. FIG. 7D shows OCT scores 7-weeks and 14-weeks post-injection of AAV-hLIF. FIG. 7E shows representative data of a-wave and b-wave detection. FIG. 7F shows a-wave amplitude measured 7-weeks and 14-weeks post-injection of AAV-hLIF.

FIG. 8 shows AAV delivery of hLIF protects the retina from light damage. Top: Representative OCT image showing how the outer nuclear layer (ONL) thickness is measured. Thickness was measured from superior to inferior retina 500 μm from the optic nerve. Bottom: Spider plot of the ONL thickness of light damaged retinas treated with AAV-CBA-hLIF. No treatment and mock injected were used as controls.

FIGS. A-9C show characterization of AAV-CBA-hLIF delivery in the mouse retina.

FIG. 9A shows scoring of retinas with high titer AAV-CBA-hLIF. FIG. 9B shows levels of hLIF in the retina determined by ELISA. Between each serotype, levels of hLIF were equivalent. FIG. 9C shows activation of STAT3 as determined by ELISA.

FIGS. 10A-10D show iterations of RPF2 development. FIG. 10A is a schematic of CBA-RPF iterations with no or one furin cleavage site (FCS). FIG. 10B shows levels of cytokine secreted by rMC-1 transfected cells into the conditioned media detected by ELISA. FIG. 10C shows activation of STAT3 in rMC-1 cells treated with conditioned media. FIG. 10D shows dose-dependent stabilization and maturation of RPF2 by TMP in transfected rMC-1 cells.

FIGS. 11A-11D show in vitro characterization of RPF2 TMP-dependent stability. FIG. 11A is a schematic depicting one embodiment of a CBA-RPF2 rAAV vector. FIG. 11B is a schematic depicting stabilization of the RPF2 fusion protein in the presence of TMP. FIG. 11C shows cytokine secretion induced by TMP-stabilization of RPF2. FIG. 11D shows pSTAT3 activation induced by TMP-stabilization of RPF2.

FIGS. 12A-12D show RPF2 is regulated in a dose-dependent manner and demonstrates no toxicity in vivo. FIG. 12A shows cytokine stabilization increases in a TMP-dose-dependent manner. FIG. 12B is a schematic depicting dosing schedule and sample analysis. FIG. 12C shows ONL thickness measured 7-weeks and 14-weeks post-administration of rAAV-CBA-RPF2. FIG. 12D shows a-wave amplitude measured 7-weeks and 14-weeks post-administration of rAAV-CBA-RPF2.

FIG. 13 shows characterization of RPF2 in vivo. Top panel shows levels of RPF2 in the retina of animals injected with the high titer dose. Bottom panel shows levels of RPF2 in the retina when TMP is administered in the food. Cytokine levels were detected by ELISA after one week of TMP treatment.

FIG. 14 shows mouse daily food intake is unchanged with TMP supplemented chow. Food intake was measured for one week for mice on normal and TMP chow.

FIGS. 15A-15C show RPF2 reduces retinal degeneration in an acute light damage mouse model. FIG. 15A is a schematic depicting dosing and data analysis schedules. FIG. 15B shows ONL thickness before and after Light Damage (LD) in control mice and mice administered AAV-CBA-RPF2. FIG. 15C shows a-wave amplitude measured before and after Light Damage (L)) in control mice and mice administered AAV-CBA-RPF2.

FIG. 16 shows RPF2 treated retinas show improved retinal structure and function after bright light exposure. Top panel shows averaged thickness of the retinal outer nuclear layer and retinal function by ERG after RPF2 stabilization in high titer treated animals. Bottom panel shows low titer and high titer RPF2-treated animals showed less signs of edema, rosette formation and infiltrating immune cells compared to the control groups.

FIGS. 17A-17D show RPF2 preserves cone morphology in the inherited rd10 retinal degeneration model. FIG. 17A is a schematic depicting a timeline of photoreceptor death and administration of AAV-CBA-RPF2. FIG. 17B shows ONL thickness measured between 3-weeks and 8-weeks of age. FIG. 17C shows preservation of cones in AAV-CBA-RPF2-injected mice. FIG. 17D shows cone number per 10 μm² in mock and AAV-CBA-RPF2-injected mice.

FIGS. 18A-18C show RPF2-treated rd10 mice maintain long-term cone-dependent vision. FIG. 18A shows b-wave amplitude measured between 3 and 8 weeks of age. FIG. 18B is a schematic depicting an OptoMotry assay. FIG. 18C shows visual acuity measurement in RPF2-treated rd10 mice.

FIG. 19 is a schematic depicting one embodiment of a plasmid comprising an rAAV vector encoding ASCL1 fused to a destabilization domain (DD). Presence of the DD allows for regulation of expression by TMP.

FIG. 20 is a schematic depicting one embodiment of a plasmid comprising an rAAV vector encoding CNTF fused to a destabilization domain (DD). Presence of the DD allows for regulation of expression by TMP.

FIG. 21 is a schematic depicting one embodiment of a plasmid comprising an rAAV vector encoding Cre recombinase fused to a destabilization domain (DD). Presence of the DD allows for regulation of expression by TMP.

FIG. 22 is a schematic depicting one embodiment of a plasmid comprising an rAAV vector encoding EDN2 fused to a destabilization domain (DD). Presence of the DD allows for regulation of expression by TMP.

FIG. 23 is a schematic depicting one embodiment of a plasmid comprising an rAAV vector encoding HBEGF fused to a destabilization domain (DD). Presence of the DD allows for regulation of expression by TMP.

FIG. 24 is a schematic depicting one embodiment of a plasmid comprising an rAAV vector encoding NRF2 fused to a destabilization domain (DD). Presence of the DD allows for regulation of expression by TMP.

FIG. 25 is a schematic depicting one embodiment of a plasmid comprising an rAAV vector encoding OSM fused to a destabilization domain (DD). Presence of the DD allows for regulation of expression by TMP.

FIG. 26 is a schematic depicting one embodiment of a plasmid comprising an rAAV vector encoding OSM-M2 fused to a destabilization domain (DD). Presence of the DD allows for regulation of expression by TMP.

FIG. 27 is a schematic depicting one embodiment of a plasmid comprising an rAAV vector encoding STAT3 fused to a destabilization domain (DD). Presence of the DD allows for regulation of expression by TMP.

FIG. 28 is a schematic depicting one embodiment of a plasmid comprising an rAAV vector encoding STAT3 variant Y705F fused to a destabilization domain (DD). Presence of the DD allows for regulation of expression by TMP.

FIG. 29 is a schematic depicting one embodiment of a plasmid comprising an rAAV vector encoding STAT3 variant S727E fused to a destabilization domain DD). Presence of the DD allows for regulation of expression by TMP.

FIG. 30 is a schematic depicting one embodiment of a plasmid comprising an rAAV vector encoding VEGFR2 IgG 1-3 (e.g., loops 1-3 of VEGFR2) fused to a destabilization domain (DD). Presence of the DD allows for regulation of expression by TMP.

FIG. 31 is a schematic depicting one embodiment of a plasmid comprising an rAAV vector encoding VEGFR2 IgG 1-7 (e.g., loops 1-7 of VEGFR2) fused to a destabilization domain (DD). Presence of the DD allows for regulation of expression by TMP.

FIG. 32 is a schematic depicting one embodiment of a plasmid comprising an rAAV vector encoding VEGFR2 IgG 2-3 (e.g., loops 2-3 of VEGFR2) fused to a destabilization domain (DD). Presence of the DD allows for regulation of expression by TMP.

### DETAILED DESCRIPTION

Aspects of the disclosure relate to compositions useful for delivering one or more transgenes (e.g., proteins, such as therapeutic proteins) to a subject, for example by adeno-associated virus (AAV)-based expression. The disclosure is based, in part, on expression vectors (e.g., AAV expression vectors) encoding fusion proteins comprising a destabilization domain (DD), which allows for control of protein expression in a cell (e.g., induction or inhibition) by altering the amount of an agent, such as a small molecule, that interacts with the DD. The disclosure relates, in some aspects, to methods for delivering and/or modulating expression of therapeutic transgenes in a cell or subject.

### Isolated Nucleic Acids and Transgenes

In some aspects, the disclosure relates to isolated nucleic acids encoding one or more proteins (e.g., therapeutic proteins), or a portion thereof. Generally, an isolated nucleic acid may encode 1, 2, 3, 4, 5, or more than 5 (e.g., 10, 15, etc.) proteins, or portions thereof. A “portion” of a protein refers to a truncated amino acid sequence that retains the intended function of the full-length protein from which it is derived. A portion of a protein can be truncated at the N-terminus. C-terminus, or N- and C-termini relative to the protein from which the portion is derived. In some embodiments, a portion of a protein comprises an amino acid sequence comprising at least 20%, at least 30%, at least 40%, at least 50%, at least 60%, at least 70%, at least 80%, at least 90%, at least 95%, or at least 99% of the amino acids present in the protein from which the portion is derived.

In some embodiments, an isolated nucleic acid encodes a fusion protein. As used here, “fusion protein” refers to a protein that is produced by connecting two or mom polypeptides which are derived from separate proteins to form a single, continuous polypeptide. In some embodiments, a fusion protein is produced by chemical conjugation (e.g., with or without a linker) between two polypeptides derived from separate proteins.

In some aspects, the disclosure relates to isolated nucleic acids comprising a transgene that encodes a protein selected from a neurotrophic factor, transcription factor, growth factor, protective factor, and a growth factor receptor subunit. Examples of neurotrophic factors include leukemia-inhibitory factor (LIF.). Ciliary neurotrophic factor (CNTF), Endothelin 2 (EDN2), and Oncostatin M (OSM). Examples of transcription factors include ASCL1, NRF2. STAT3, etc. Examples of growth factors include VEGF. HBEGF, etc.

In some embodiments, a fusion protein encodes a Ciliary neurotrophic factor (CNTF) protein or a fragment thereof. CNTF is a polypeptide hormone and neurotrophic factor that promotes neurotransmitter synthesis and neurite outgrowth. Human CNTF is encoded by the CNTF gene, for example as set forth in NCBI Reference Sequence No. NM_000614. In some embodiments, a CNTF protein comprises the amino acid sequence set forth in SEQ ID NO: 7. In some embodiments, a CNTF protein comprises an amino acid sequence that is at least 70%, at least 80%, at least 90%, at least 95%, or at least 99% identical to the amino acid sequence set forth in SEQ ID NO: 7. In some embodiments, a CNTF protein is encoded by a nucleic acid sequence that encodes an amino acid sequence that is at least 70%, at least 80%, at least 90%, at least 95%, or at least 99% identical to the amino acid sequence set forth in SEQ ID NO: 7.

In some embodiments, a fusion protein encodes an Oncostatin M (OSM) protein or a fragment thereof. Oncostatin M is a pleiotropic cytokine that is part of the IL6 cytokine group, and signals through gp130 cell surface receptors (e.g., cell surface receptors comprising gp130 and LIFR or OSMR). Human OSM is encoded by the OSM gene, for example as set forth in NCBI Reference Sequence No. NM_020530. In some embodiments, an OSM protein comprises the amino acid sequence set forth in SEQ ID NO: 17. In some embodiments, a fusion protein encodes a variant of OSM protein, for example OSM M2 protein (e.g., as described by Chollangi et al. (2012) J Biol Chem 287(39):32848-32859). In some embodiments, an OSM M2 protein comprises the amino acid sequence set forth in SEQ ID NO: 19. In some embodiments, an OSM protein comprises an amino acid sequence that is at least 70%, at least 80%, at least 90%, at least 95%, or at least 99% identical to the amino acid sequence set forth in SEQ ID NO: 17 or 19. In some embodiments, a OSM protein is encoded by a nucleic acid sequence that encodes an amino acid sequence that is at least 70%, at least 80%, at least 90%, at least 95%, or at least 99% identical to the amino acid sequence set forth in SEQ ID NO: 17 or 19.

In some embodiments, a fusion protein encodes a Endothelin 2 (EDN2) protein or a fragment thereof. Endothelin 2 (EDN2) protein is a member of the endothelin protein family of secretory vasoconstrictive peptides, and functions as a ligand for endothelin receptors that initiate intracellular signaling events. Human EDN2 is encoded by the EDN2 gene, for example as set forth in NCBI Reference Sequence No. NM_001956. In some embodiments, an EDN2 protein comprises the amino acid sequence set forth in SEQ ID NO: 11. In some embodiments, a EDN2 protein comprises an amino acid sequence that is at least 70%, at least 80%, at least 90%, at least 95%, or at least 99% identical to the amino acid sequence set forth in SEQ ID NO: 11. In some embodiments, a EDN2 protein is encoded by a nucleic acid sequence that encodes an amino acid sequence that is at least 70%, at least 80%, at least 90%, at least 95%, or at least 99% identical to the amino acid sequence set forth in SEQ ID NO: 11.

In some embodiments, a fusion protein encodes an Achaete-scute homolog 1 (ASCL1) protein or a fragment thereof. Achaete-scute homolog 1 is protein that is a member of the basic helix-loop-helix (BHLH) family of transcription factors. ASCL1 plays a role in the neuronal commitment and differentiation and in the generation of olfactory and autonomic neurons. Human ASCL1 is encoded by the ASCL1 gene, for example as set forth in NCBI Reference Sequence No. NM_004307. In some embodiments, an ASCL1 protein comprises the amino acid sequence set forth in SEQ ID NO: 5. In some embodiments, a ASCL1 protein comprises an amino acid sequence that is at least 70%, at least 80%, at least 90%, at least 95%, or at least 99% identical to the amino acid sequence set forth in SEQ ID NO: 5. In some embodiments, a ASCL1 protein is encoded by a nucleic acid sequence that encodes an amino acid sequence that at least 70%, at least 80%, at least 90%, at least 95%, or at least 99% identical to the amino acid sequence set forth in SEQ ID NO: 5.

In some embodiments, a fusion protein encodes a Nuclear factor (erythroid-derived 2)-like 2 growth factor (NRF2) protein or a fragment thereof. NFR2 is transcription factor (a basic leucine zipper (bZIP) protein) that regulates the expression of antioxidant proteins that protect against oxidative damage triggered by injury and inflammation. Human NFR2 is encoded by the NFR2 gene, for example as set forth in NCBI Reference Sequence No. NM_006164. In some embodiments, an NRF2 protein comprises the amino acid sequence set forth in SEQ ID NO: 15. In some embodiments, a NRF2 protein comprises an amino acid sequence that is at least 70%, at least 80%, at least 90%, at least 95%, or at least 99% identical to the amino acid sequence set forth in SEQ ID NO: 15. In some embodiments, a NRF2 protein is encoded by a nucleic acid sequence that encodes an amino acid sequence that is at least 70%, at least 80%, at least 90%, at least 95%, or at least 99% identical to the amino acid sequence set forth in SEQ ID NO: 15.

In some embodiments, a fusion protein encodes a Signal transducer and activator of transcription 3 (STAT3) protein or a fragment thereof, STAT3 is a transcription factor that is phosphorylated in response to cytokines and growth factors, and plays important roles in regulation of gene expression during cell growth and apoptosis. Human STAT3 is encoded by the STAT3 gene, for example as set forth in NCBI Reference Sequence No. NM_003150. In some embodiments, an STAT3 protein comprises the amino acid sequence set forth in SEQ ID NO: 21. In some embodiments, a STAT3 protein comprises an amino acid sequence that is at least 70%, at least 80%, at least 90%, at least 95%, or at least 99% identical to the amino acid sequence set forth in SEQ ID NO: 21. In some embodiments, a STAT3 protein is encoded by a nucleic acid sequence that encodes an amino acid sequence that is at least 70%, at least 80%, at least 90%, at least 95%, or at least 99% identical to the amino acid sequence set forth in SEQ ID NO: 21.

In some embodiments, a fusion protein encodes a Signal transducer and activator of transcription 3 (STAT3) protein variant (e.g., STAT3 Y705F, STAT3 S727E, etc.) or a fragment thereof. A variant of STAT3 may have one or more (e.g., 2, 3, 4, 5, 6, 7, 8, 9, 10, or more) amino acid substitutions relative to a wild-type STAT3 protein (e.g., SEQ ID NO: 21). In some embodiments, at least one of the mutations results in the variant functioning as a phosphomimetic of STAT3 (e.g., the variant comprises one or more amino acid substitutions that mimic phosphorylation of the wild-type protein). In some embodiments, an STAT3 protein variant comprises an amino acid mutation at position 705 (e.g., Y705D) or position 772 (e.g., S727E). In some embodiments, a STAT3 protein variant comprises the amino acid sequence set forth in SEQ ID NO: 23 or 25.

In some embodiments, a fusion protein encodes a Heparin-binding EGF-like growth factor (HBEGF) protein or a fragment thereof. Heparin-binding EGF-like growth factor is a membrane-anchored mitogenic and chemotactic glycoprotein, and plays a role in wound healing, cardiac hypertrophy, and heart development and function. Human HBEGF is encoded by the HBEGF gene, for example as set forth in NCBI Reference Sequence No. NM_001945. In some embodiments, an HBEGF protein comprises the amino acid sequence set forth in SEQ ID NO: 13. In some embodiments, a HBEGF protein comprises an amino acid sequence that is at least 70%, at least 80%, at least 90%, at least 95%, or at least 99% identical to the amino acid sequence set forth in SEQ ID NO: 13. In some embodiments, a HBEGF protein is encoded by a nucleic acid sequence that encodes an amino acid sequence that is at least 70%, at least 80%, at least 90%, at least 95%, or at least 99% identical to the amino acid sequence set forth in SEQ ID NO: 13.

In some embodiments, a fusion protein encodes a vascular endothelial growth factor receptor 2 (VEGFR2) protein or a fragment thereof. Vascular endothelial growth factor receptor 2 is a tyrosine kinase receptor that functions on the cell surface, and plays a role in cell mitogenesis and cell migration. Human VEGFR2 is encoded by the VEGFR gene, for example as set forth in NCBI Reference Sequence No. NM_002253. VEGFR2 proteins typically comprise an extracellular portion consisting of 7 immunoglobulin-like (Ig) domains, a single transmembrane spanning region and an intracellular portion containing a split tyrosine-kinase domain. In some embodiments, a fusion protein encodes a truncated VEGFR2 protein, for example a protein that comprises one or more Ig-like loop regions, for example Ig-like regions 1-3, Ig-like regions 1-7. Ig-like regions 2-3, etc. of VEGFR2 but not other regions (e.g., the transmembrane spanning region). In some embodiments, an VEGFR2 protein comprises the amino acid sequence set forth in any one of SEQ ID NOs: 27, 29, and 31. In some embodiments, a VEGFR2 protein comprises an amino acid sequence that is at least 70%, at least 80%, at least 90%, at least 95%, or at least 99% identical to the amino acid sequence set forth in SEQ ID NO: 27. In some embodiments, a VEGFR2 protein is encoded by a nucleic acid sequence that encodes an amino acid sequence that is at least 70%, at least 80%, at least 90%, at least 95%, or at least 99% identical to the amino acid sequence set forth in SEQ ID NO: 27.

In some embodiments, a fusion protein encodes a Cre recombinase (Cre) protein or a fragment thereof. In some embodiments, an Cre protein comprises the amino acid sequence set forth in SEQ ID NO: 9. In some embodiments, a Cre protein comprises an amino acid sequence that is at least 70%, at least 80%, at least 90%, at least 95%, or at least 99% identical to the amino acid sequence set forth in SEQ ID NO: 9. In some embodiments, a Cre protein is encoded by a nucleic acid sequence that encodes an amino acid sequence that is at least 70%, at least 80%, at least 90%, at least 95%, or at least 99% identical to the amino acid sequence set forth in SEQ ID NO: 9.

The disclosure is based, in part, on the discovery that unregulated expression of certain proteins (e.g., growth factors, transcription factors, neurotrophic factors, etc.) in cells leads, in some embodiments, to cellular damage. Accordingly, in some embodiments, isolated nucleic acids described by the disclosure (e.g., isolated nucleic acids encoding proteins described herein) comprise one or more control elements that allow for regulation of gene expression (e.g., expression of ASCL1, CNTF, EDN2, HBEGF, NRF2, OSM, OSM-M2, STAT3, STAT3 Y705F, STAT3 S727E, Cre recombinase, VEGFR2, etc.). Non-limiting examples of control elements include promoters, insulators, silencers, response elements, introns, enhancers, initiation sites, termination signals, and poly(A) tails. Any combination of such control sequences is contemplated herein (e.g., a promoter and an enhancer).

In some embodiments, one or more promoters may be operably linked to a coding nucleotide sequence in the heterologous nucleic acid. A promoter is “operably linked” to a nucleotide sequence when the promoter sequence controls and/or regulates the transcription of the nucleotide sequence. A promoter may be a constitutive promoter, tissue-specific promoter, an inducible promoter, or a synthetic promoter. In some embodiments, a promoter comprises a β-actin promoter (e.g., chicken β-actin promoter. CBA. In some embodiments, a promoter is a chimeric viral/mammalian promoter, for example a chimeric CMV/chicken beta actin (CBA. CB or CAG) promoter.

The disclosure relates, in some aspects, to isolated nucleic acids encoding fusion proteins that comprise a destabilization domain (DD). As used herein, a “destabilization domain” or “DD” refers to a nucleic acid sequence encoding a protein (or a portion thereof) that, when fused to a sequence encoding a second protein, reversibly alters the stability of (e.g., destabilizes) the entire fusion protein (e.g., the DD and the second protein). Generally, the instability of the second protein caused by fusion to a DD can be reversed by addition of a small molecule that binds to the DD and re-stabilizes the fusion protein. The use of DDs to regulate protein expression from transgenes is known and described for example by Iwamoto et al. (2010) Chem Biol. 17(9)981-988. Non-limiting examples of DDs include but are not limited to FK506 binding protein (FKBP) destabilization domains and dihydrofolate reductase (DHFR) destabilization domains. In some embodiments, a DD comprises a sequence encoding a dihydrofolate reductase (DHFR) protein, or a fragment thereof.

Dihydrofolate reductase (DHFR) is an enzyme that catalyzes reduction of dihydrofolate to tetrahydrofolate. It has been observed that, in mammalian cells, fusion proteins containing bacterial DHFR protein (e.g., E. coli DHFR protein) are rapidly ubiquitinated and degraded by the proteasome system. However, binding of certain inhibitors of bacterial DHFR results in stabilization of the molecule and prevents degradation of the protein. Non-limiting examples of DHFR inhibitors include but are not limited to diaminoquinazolines, diaminopyrroloquinazolines, diaminopyrimidines, diaminopteridines, diaminotriazines, JP2056, piritrexim, cycloguanil, trimethoprim (TMP), iclaprim, tetrotoxoprim, etc. In some embodiments, an isolated nucleic acid encoding a fusion protein comprises a sequence encoding a DD (e.g., DHFR protein or a fragment thereof) is capable of being bound by trimethoprim (TMP).

Fusion of a DD directly to a protein, in some embodiments, prevents transit of the protein through cellular secretion pathways. The disclosure is based, in part, on the recognition that inclusion of a linker sequence between a sequence encoding a protein (e.g., a LIF protein) and a DD, in some embodiments, results in improved protein secretion. Thus, in some embodiments, a nucleic acid sequence encoding a protein (e.g., an LIF protein) is linked to a nucleic acid sequence encoding a DD by a linker sequence.

In some embodiments, a linker sequence encodes a peptide spacer, for example a glycine-rich and/or serine-rich peptide (e.g., GGGGS, (GGGGS)ₓ, where x is an integer between 1 and 20, etc.). The length of a linker can vary, for example from about 1 amino acid to about 500 amino acids (e.g., any integer between 1 and 500, inclusive). In some embodiments, a linker sequence comprises a cleavable linker sequence. Non-limiting examples of cleavable linkers include enzyme (e.g., protease) cleavable linkers and photocleavable linkers and are described for example in Leriche et al. (2012) Bioorg Med Chem 20(2):571-82. Examples of protease cleavable linkers include amino acid sequences that are substrates for cathepsin (e.g., cathepsin B), matrix metalloproteases (e.g., MMP1, MMP9, etc.), and furin. Generally, the number of protease cleavage substrates can vary. In some embodiments, a linker sequence encodes 1, 2, 3, 4, 5, 6, 7, 8, 9 or 10 protease cleavage sites. In some embodiments, the one or more protease cleavage sites are furin cleavage sites.

Furin is a member of the subtilisin-like proprotein convertase family and generally functions to process precursor proteins to their active forms by cleaving at a conserved dibasic amino acid consensus motif. In some embodiments, furin cleaves a sequence comprising RX(K/R)R, where X is any amino acid. Furin cleavage sites are known and described, for example by Tian et al. (2011) Int. J Mol Sci. 12(2):1060-1065.

The disclosure relates, in part, to recombinant adeno-associated virus vectors (rAAV vectors) comprising an isolated nucleic acid as described herein. Generally, an rAAV vector is an isolated nucleic acid sequence comprising an expression cassette engineered to express a transgene (e.g., an isolated nucleic acid encoding a LIF protein, and optionally, one or more furin cleavage sites and/or a DD) that is flanked by AAV inverted terminal repeat (ITR) sequences. The ITR sequences can be derived from any AAV serotype, including but not limited to AAV2, AAV3, AAV4, AAV5, AAV6, AAV7, AAV8, AAV9. In some embodiments, the AAV ITR sequences are AAV2 ITR sequences. In some embodiments, one of the ITR sequences in a rAAV vector is a truncated ITR (e.g., a ΔTRS ITR), which in some embodiments are useful for production of self-complementary rAAVs (scAAVs). In some embodiments, an rAAV vector is part of a plasmid (e.g., a bacterial plasmid).

In some embodiments, an isolated nucleic acid comprises AAV ITR sequences flanking a transgene that encodes a fusion protein (e.g., a fusion protein comprising a DD). ITR sequences and plasmids containing ITR sequences are known in the art and are commercially available (see, e.g., products and services available from Vector Biolabs. Philadelphia, PA; Cellbiolabs. San Diego, CA; Agilent Technologies, Santa Clara, Ca; and Addgene, Cambridge. MA; and Gene delivery to skeletal muscle results in sustained expression and systemic delivery of a therapeutic protein. Kessler P D, Podsakoff G M, Chen X, McQuiston S A, Colosi P C, Matelis L A, Kurtzman G J, Byrne BJ, Proc Natl Acad Sci USA. 1996 Nov. 26; 93(24):14082-7; and Curtis A. Machida. Methods in Molecular Medicine™. Viral Vectors for Gene Therapy Methods and Protocols. 10.1385/1-59259-304-6:201® Humana Press Inc. 2003. Chapter 10. Targeted Integration by Adeno-Associated Virus. Matthew D. Weitzman. Samuel M. Young Jr., Toni Cathomen and Richard Jude Samulski; U.S. Pat. Nos. 5,139,941 and 5,962,313, all of which are incorporated herein by reference).

### Retinal Protective Factors

In some aspects, the disclosure relates to compositions and methods useful for maintaining or improving retinal function and/or morphology. The disclosure is based, in part, on isolated nucleic acids encoding certain neurotrophic factors (e.g., leukemia inhibitory factor (LIF), etc.) and gene therapy vectors (e.g., recombinant adeno-associated virus (rAAV) vectors) encoding the same. In some embodiments, isolated nucleic acids and gene therapy vectors described by the disclosure are useful for treatment of certain diseases or disorders of the eye, for example retinal degeneration, retinitis pigmentosa (RP), age-related macular degeneration (AMD), glaucoma, etc.

In some aspects, the disclosure provides isolated nucleic acids encoding transgenes encoding molecules (e.g., proteins, interfering nucleic acids, etc.) that bind to receptors located in the eye of a subject. In some embodiments, an isolated nucleic acid described by the disclosure encodes a protein, for example a protein that binds to a receptor. Generally, a protein that binds to a receptor can bind to a G-protein coupled receptor (GPCR), an ion channel (e.g., a ligand-gated ion channel), a cytokine receptor, an enzyme-linked receptor, an intracellular receptor, etc.

In some embodiments, a protein as described by the disclosure binds to a cytokine receptor. Examples of cytokine receptors include but are not limited to Type I cytokine receptors. Type II cytokine receptors, Immunoglobulin receptors. Tumor Necrosis Factor receptors, chemokine receptors, etc. In some embodiments, nucleic acid encodes a protein that binds to a Type I cytokine receptor, for example a cytokine receptor comprising a gp130 subunit and/or a leukemia inhibitory factor receptor (LIFR) subunit. Type I cytokine receptors comprising LIFR subunits are generally know and am described, for example, in Giese et al. (2005) J. Cell Sci 118:5129-5140.

Typically, proteins that bind to LIFRs are IL-6 type cytokines, including but not limited to Leukemia inhibitory factor (LIF), ciliary neurotrophic factor (CNTF), Cardiotrophin-1 (CT-1). Eosinophil lysophospholipase (CLC), oncostatin M (OSM), etc. Thus, in some embodiments, a nucleic acid described by the disclosure encodes a protein selected from LIF, CNTF, CT-1, CLC, OSM, or a portion thereof.

A “portion” of a protein refers to a truncated amino acid sequence that retains the intended function of the full-length protein from which it is derived. For example, a truncated IL-6 type cytokine, in some embodiments, retains the function of binding to receptors comprising a gp130 subunit and/or a LIFR subunit. A portion of a protein can be truncated at the N-terminus. C-terminus, or N- and C-termini relative to the protein from which the portion is derived. In some embodiments, a portion of a protein comprises an amino acid sequence comprising at least 20%, at least 30%, at least 40%, at least 50%, at least 60%, at least 70%, at least 80%, at least 90%, at least 95%, or at least 99% of the amino acids present in the protein from which the portion is derived.

In some embodiments, an isolated nucleic acid encodes a Leukemia inhibitory factor (LIF) protein, or a portion thereof. LIF is an IL-6 type cytokine that is generally involved with control of cell differentiation and signaling through the Jak/STAT pathway and the MAPK signaling cascade. In some embodiments, the LIF is mammalian LIF, for example human LIF (e.g., hLIF) or mouse LIF. In some embodiments. LIF comprises a sequence set forth in SEQ ID NO: 1. In some embodiments, an isolated nucleic acid described by the disclosure comprises a sequence that is that is at least 75% (e.g., at least 75%, at least 80%, at least 85%, at least 90%, at least 95%, or at least 99%) identical to SEQ ID NO: 1. In some embodiments, an isolated nucleic acid described by the disclosure comprises a sequence that encodes a codon-optimized LIF. Codon optimization is well-known in the art and is described, for example by Quax et al. (2015) Molecular Cell 59:149-161.

The disclosure is based, in part, on the discovery that unregulated expression of LIF proteins in the eye leads, in some embodiments, to cellular damage of retinal cells. Accordingly, in some embodiments, isolated nucleic acids described by the disclosure (e.g., isolated nucleic acids encoding LIF or a portion thereof) comprise one or more control elements that allow for regulation of gene expression (e.g., expression of LIF). Non-limiting examples of control elements include promoters, insulators, silencers, response elements, introns, enhancers, initiation sites, termination signals, and poly(A) tails. Any combination of such control sequences is contemplated herein (e.g., a promoter and an enhancer).

In some embodiments, one or more promoters may be operably linked to a coding nucleotide sequence in the heterologous nucleic acid. A promoter is “operably linked” to a nucleotide sequence when the promoter sequence controls and/or regulates the transcription of the nucleotide sequence. A promoter may be a constitutive promoter, tissue-specific promoter, an inducible promoter, or a synthetic promoter.

For example, constitutive promoters of different strengths can be used. A nucleic acid described herein may include one or more constitutive promoters, such as viral promoters or promoters from mammalian genes that are generally active in promoting transcription. Non-2) limiting examples of constitutive viral promoters include the Herpes Simplex virus (HSV), thymidine kinase (TK). Rous Sarcoma Virus (RSV). Simian Virus 40 (SV40). Mouse Mammary Tumor Virus (MMTV). Ad E1A cytomegalovirus (CMV) promoters. Non-limiting examples of constitutive mammalian promoters include various housekeeping gene promoters, as exemplified by the β-actin promoter (e.g., chicken β-actin promoter) and human elongation factor-1α (EF-1α) promoter. In some embodiments, chimeric viral/mammalian promoters may include a chimeric CMV/chicken beta actin (CBA, CB or CAG) promoters.

Inducible promoters and/or regulatory elements may also be contemplated for achieving appropriate expression levels of the protein or polypeptide of interest. Non-limiting examples of suitable inducible promoters include those from genes such as cytochrome P450 genes, heat shock protein genes, metallothionein genes, and hormone-inducible genes, such as the estrogen gene promoter. Another example of an inducible promoter is the tetVP16 promoter that is responsive to tetracycline.

Tissue-specific promoters and/or regulatory elements are also contemplated herein. In some embodiments, it may be beneficial to combine an isolated nucleic acid (e.g., an isolated nucleic acid encoding LIF protein) as disclosed herein, with a promoter that targets the cells, tissue, or organ where expression of LIF is desired. For example, if LIF expression is desired in the eye of a subject, a nucleic acid may comprise a promoter that targets photoreceptor cells or the retina as a whole. In some embodiments, a cell-type-specific promoter targeting the retina is human rhodopsin kinase promoter (hGRK1). Non-limiting examples of hGRK1 promoter can be found in Beltran et al., 2010, Gene Ther. 17:1162, Zolotukhin et al., 2005. Hum Gene Ther. 16:551, and Jacobson et al., Mol Ther. 13:1074. In some embodiments, a retina-specific promoter is a Pleiades Mini-promoter (for example Ple155). In some embodiments, a retina-specific promoter is glial fibrillary acidic protein promoter. Other non-limiting examples of promoters that can be used as retinal cell-type-specific promoters include red opsin promoter “PR2.1” (which targets M and L cones), chimeric ‘IRBPe-GNAT2’ promoter (which targets all cones), IRBP promoter (which targets rods). Grm6-SV40 enhancer/promoter (which targets bipolar cells). Thy1 (which targets RGCs), other Pleiades promoters, rod opsin promoter (which targets rods), cone arrestin promoters (which targets all cones), VMD2 or Bestrophin promoter (which targets RPE cells).

Several promoters are publically available or described. For example. Ple155 promoter is available through Addgene plasmid repository (Addgene plasmid #29011, addgene.org/29011/) and is described in Scalabrino et al. (Hum Mol Genet. 2015, 24(21):6229-39). Ye et al. (Hum Gene Ther.; 27(1):72-82) describes a shorter version of this promoter called PR1.7. A Thy1 promoter construct is also available through Addgene plasmid repository (Addgene plasmid #20736, addgene.org/20736/). A GRM6 promoter construct is also available through Addgene plasmid repository (Addgene plasmid #66391, addgene.org/66391/). Guziewicz et al. (PLoS One. 2013 Oct. 15; 8(10):e75666) and Esumi et al (J Biol Chem. 2004, 279(18):19064-73) provide examples of the use of VMD2 promoter. Dyka et al. (Adv Exp Med Biol. 2014; 801: 695-701) describes cone specific promoters for use in gene therapy, including IRBP and IRBPe-GNAT2 promoter. The use of PR2.1 promoter has been demonstrated in Komáromy et al. (Gene Ther. 2008 July; 15(14):1049-55) and its characterization in Karim et al. (Tree Physiol. 2015 October; 35(10):1129-39). Aartsen et al. (PLoS One, 5(8):e12387) describes the use of GFAP promoter to drive GFP expression in Muller glial cells. Other examples of Muller glia specific promoters are RLBP1 and GLAST (Vázquez-Chona, Invest Ophthalmol Vis Sci. 2009, 50(8):3996-4003; Regan et al., Journal of Neuroscience, 2007, 27(25): 6607-6619).

Synthetic promoters are also contemplated herein. A synthetic promoter may comprise, for example, regions of known promoters, regulatory elements, transcription factor binding sites, enhancer elements, repressor elements, and the like.

It is to be understood that a promoter may be a fragment of any one of the promoters disclosed herein, or one that retains partial promoter activity (e.g., 10-90, 30-60, 50-80.80-99 or 90-99.9% of the activity) of a whole promoter.

The disclosure relates, in some aspects, to isolated nucleic acids encoding LIF proteins and further comprising a destabilization domain (DD). As used herein, a “destabilization domain” or “DD” refers to a nucleic acid sequence encoding a protein (or a portion thereof) that, when fused to a sequence encoding a second protein, reversibly alters the stability of (e.g., destabilizes) the entire fusion protein (e.g., the DD and the second protein). Generally, the instability of the second protein caused by fusion to a DD can be reversed by addition of a small molecule that binds to the DD and re-stabilizes the fusion protein. The use of DDs to regulate protein expression from transgenes is known and described for example by Iwamoto et al. (2010) Chem Biol. 17(9)981-988. Non-limiting examples of DDs include but are not limited to FK506 binding protein (FKBP) destabilization domains and dihydrofolate reductase (DHFR) destabilization domains. In some embodiments, a DD comprises a sequence encoding a dihydrofolate reductase (DHFR) protein, or a fragment thereof.

Dihydrofolate reductase (DHFR) is an enzyme that catalyzes reduction of dihydrofolate to tetrahydrofolate. It has been observed that, in mammalian cells, fusion proteins containing bacterial DHFR protein (e.g., E. coli DHFR protein) are rapidly ubiquitinated and degraded by the proteasome system. However, binding of certain inhibitors of bacterial DHFR results in stabilization of the molecule and prevents degradation of the protein. Non-limiting examples of DHFR inhibitors include but are not limited to diaminoquinazolines, diaminopyrroloquinazolines, diaminopyrimidines, diaminopteridines, diaminotriazines. JP2056, piritrexim, cycloguanil, trimethoprim (TMP), iclaprim, tetrotoxoprim, etc. In some embodiments, an isolated nucleic acid encoding LIF comprises a sequence encoding a DD (e.g., DHFR protein or a fragment thereof) is capable of being bound by trimethoprim (TMP).

Fusion of a DD directly to a LIF protein, in some embodiments, prevents transit of the LIF protein through cellular secretion pathways. The disclosure is based, in part, on the recognition that inclusion of a linker sequence between a sequence encoding a protein (e.g., a LIF protein) and a DD, in some embodiments, results in improved LIF protein secretion. Thus, in some embodiments, a nucleic acid sequence encoding a protein (e.g., an LF protein) is linked to a nucleic acid sequence encoding a DD by a linker sequence.

In some embodiments, a linker sequence encodes a peptide spacer, for example a glycine-rich and/or serine-rich peptide (e.g., GGGGS, (GGGGS)ₓ, where x is an integer between 1 and 20, etc.). The length of a linker can vary, for example from about 1 amino acid to about 500 amino acids (e.g., any integer between 1 and 500, inclusive). In some embodiments, a linker sequence comprises a cleavable linker sequence. Non-limiting examples of cleavable linkers include enzyme (e.g., protease) cleavable linkers and photocleavable linkers and are described for example in Leriche et al. (2012) Bioorg Med Chem 20(2):571-82. Examples of protease cleavable linkers include amino acid sequences that are substrates for cathepsin (e.g., cathepsin B), matrix metalloproteases (e.g., MMP1, MMP9, etc.), and furin. Generally, the number of protease cleavage substrates can vary. In some embodiments, a linker sequence encodes 1, 2, 3, 4, 5, 6, 7, 8, 9 or 10 protease cleavage sites. In some embodiments, the one or more protease cleavage sites are furin cleavage sites.

Furin is a member of the subtilisin-like proprotein convertase family and generally functions to process precursor proteins to their active forms by cleaving at a conserved dibasic amino acid consensus motif. In some embodiments, furin cleaves a sequence comprising RX(K/R)R, where X is any amino acid. Furin cleavage sites are known and described, for example by Tian et al. (2011) Int. J Mol Sci. 12(2):1060-1065.

The disclosure relates, in part, to recombinant adeno-associated virus vectors (rAAV vectors) comprising an isolated nucleic acid as described herein. Generally, an rAAV vector is an isolated nucleic acid sequence comprising an expression cassette engineered to express a transgene (e.g., an isolated nucleic acid encoding a LIF protein, and optionally, one or more furin cleavage sites and/or a DD) that is flanked by AAV inverted terminal repeat (ITR) sequences. The ITR sequences can be derived from any AAV serotype, including but not limited to AAV2, AAV3, AAV4, AAV5, AAV6, AAV7, AAV8, AAV9. In some embodiments, the AAV ITR sequences are AAV2 ITR sequences. In some embodiments, one of the ITR sequences in a rAAV vector is a truncated ITR (e.g., a ΔTRS ITR), which in some embodiments are useful for production of self-complementary rAAVs (scAAVs). In some embodiments, an rAAV vector is part of a plasmid (e.g., a bacterial plasmid).

In some embodiments, an isolated nucleic acid described by the disclosure comprises the sequence set forth in SEQ ID NO: 3 or encodes the amino acid sequence set forth in SEQ ID NO: 4.

### Recombinant Adeno-Associated Viruses

In some aspects, the disclosure provides recombinant AAVs (rAAVs) comprising an isolated nucleic acid as described herein.

The AAV genome is built of single-stranded deoxyribonucleic acid (ssDNA), which is either positive- or negative-sensed. At each end of the DNA strand is an inverted terminal repeat (ITR). Between the ITRs are two open reading frames (ORFs); rep and cap. The rep ORF is composed of four overlapping genes encoding Rep proteins required for the AAV life cycle. The cap ORF contains overlapping nucleotide sequences of capsid proteins: VP1, VP2 and VP3, which interact together to form a capsid of an icosahedral symmetry.

The capsid proteins, which are controlled by the same promoter, designated p40, are translated from the same mRNA. The molecular weights of VP1, VP2 and VP3 are 87, 72 and 62 kiloDaltons, respectively. The AAV capsid is composed of 60 capsid protein subunits. VP1, VP2, and VP3, that are arranged in an icosahedral symmetry in a ratio of 1:1:10.

In some embodiments, a rAAV particle comprises a rAAV vector comprising an isolated nucleic acid as described herein flanked by ITRs of serotype 2. In some embodiments, a rAAV particle is a pseudotyped rAAV particle, which comprises (a) a capsid comprised of capsid proteins derived from a serotype other than serotype 2 (e.g., serotype 3, 4, 5, 6, 7, 8, 9, etc.), and (b) a rAAV vector comprising ITRs from serotype 2. For example, a particle may have ITRs of serotype 2 and a capsid of serotype 6. Such a pseudotyped rAAV particle would be designated AAV2/6.

In some embodiments, a rAAV particle comprises (a) a capsid comprised of capsid proteins derived from a serotype selected from serotype 2, 3, 4, 5, 6, 7, 8, 9, and 10, and (b) a rAAV vector comprising ITRs from serotype 2 flanking an expression cassette comprising an isolated nucleic acid as described herein.

Generally, a rAAV as described herein may comprise capsid proteins of any serotype (e.g., 2, 3, 4, 5, 6, 7, 8, 9, 10, and variants thereof, or certain non-human capsid protein serotypes, such as rh10, rh39, etc.). In some preferred embodiments, rAAV particles have one or more capsid proteins of serotype 2 or variants thereof. Examples of serotype 2 capsid variants are described, for example by Kay et al. (2013) PLoS ONE 8(4): e62097 and Boye et al. (2016) J Virol. 90(8):4215-31. In some embodiments, an rAAV comprises an AAV2 variant having one or more of the following mutations: Y272F, Y444F, Y500F, Y730F, and T491V. In some embodiments, an rAAV comprises an AAV2 variant having the following mutations: Y272F, Y444F, Y500F, Y730F, and T491V (e.g., AAV2*).

In some preferred embodiments, rAAV particles have one or more capsid proteins of serotype 6 or variants thereof. In some embodiments, an rAAV comprises an AAV6 variant having one or more of the following mutations: Y705F, Y731F, and T492V. In some embodiments, an rAAV comprises an AAV6 variant having the following mutations: Y705F, Y731F, and T492V (e.g., AAV6*).

Various methods of producing rAAV particles and nucleic acid vectors are known (see, e.g., Zolotukhin et al. Production and purification of serotype 1, 2, and 5 recombinant adeno-associated viral vectors. Methods 28 (2002) 158-167; and U.S. Patent Publication Numbers US20070015238 and US20120322861, which are incorporated herein by reference; and plasmids and kits available from ATCC and Cell Biolabs, Inc.). In some embodiments, a vector (e.g., a plasmid) comprising a gene of interest may be combined with one or more helper plasmids, e.g., that contain a rep gene (e.g., encoding Rep78, Rep68, Rep52 and Rep40) and a cap gene (encoding VP1, VP2, and VP3, including a modified VP region as described herein), and transfected into a recombinant cells, called helper or producer cells, such that the nucleic acid vector is packaged or encapsidated inside the capsid and subsequently purified.

Non-limiting examples of mammalian helper cells include HEK293 cells, COS cells. HeLa cells, BHK cells, or CHO cells (see, e.g., ATCC® CRL-1573™, ATCC® CRL-1651™, ATCC® CRL-1650™, ATCC® CCL-2, ATCC® CCL-10™, or ATCC® CCG-61™). A non-limiting example of an insect helper cells is Sf9 cells (see, e.g., ATCC® CRL-1711™). A helper cell may comprises rep and/or cap genes that encode the Rep protein and/or Cap proteins. In some embodiments, the packaging is performed in vitro (e.g., outside of a cell).

In some embodiments, a nucleic acid vector (e.g., a plasmid) containing the gene of interest is combined with one or more helper plasmids. e.g., that contain a rep gene of a first serotype and a cap gene of the same serotype or a different serotype, and transfected into helper cells such that the rAAV particle is packaged. In some embodiments, the one or more helper plasmids include a first helper plasmid comprising a rep gene and a cap gene, and a second helper plasmid comprising one or more of the following helper genes: E1a gene, E1b gene, E4 gene, E2a gene, and VA gene. For clarity, helper genes are genes that encode helper proteins E1a, E1b, E4, E2a, and VA. Helper plasmids, and methods of making such plasmids, are known in the art and commercially available (see. e.g., pDF6, pRep, pDM, pDG, pDP1rs, pDP2rs, pDP3rs, pDP4rs, pDP5rs, pDP6rs, pDG(R484E/R585E), and pDP8.ape plasmids from PlasmidFactory, Bielefeld. Germany; other products and services available from Vector Biolabs, Philadelphia, PA; Cellbiolabs. San Diego, CA; Agilent Technologies, Santa Clara, Ca; and Addgene. Cambridge. MA; pxx6; Grimm et al. (1998). Novel Tools for Production and Purification of Recombinant Adeno associated Virus Vectors, Human Gene Therapy, Vol. 9, 2745-2760; Kern, A. et al. (2003). Identification of a Heparin-Binding Motif on Adeno-Associated Virus Type 2 Capsids. Journal of Virology, Vol. 77, 11072-11081.; Grimm et al. (2003), Helper Virus-Free. Optically Controllable, and Two-Plasmid-Based Production of Adeno-associated Virus Vectors of Serotypes 1 to 6, Molecular Therapy, Vol. 7, 839-850; Kronenberg et al. (2005). A Conformational Change in the Adeno-Associated Virus Type 2 Capsid Leads to the Exposure of Hidden VP1 N Termini, Journal of Virology, Vol. 79, 5296-5303; and Moullier. P. and Snyder, R. O. (2008), International efforts for recombinant adeno-associated viral vector reference standards, Molecular Therapy, Vol. 16, 1185-1188). Plasmids that encode wild-type AAV coding regions for specific serotypes are also know and available. For example pSub201 is a plasmid that comprises the coding regions of the wild-type AAV2 genome (Samulski et al. (1987), J Virology, 6:3096-3101).

ITR sequences and plasmids containing ITR sequences are known in the art and are commercially available (see. e.g., products and services available from Vector Biolabs, Philadelphia, PA; Cellbiolabs. San Diego, CA; Agilent Technologies, Santa Clara, Ca; and Addgene, Cambridge, MA; and Gene delivery to skeletal muscle results in sustained expression and systemic delivery of a therapeutic protein. Kessler P D, Podsakoff G M, Chen X, McQuiston S A, Colosi P C, Matelis L A, Kurtzman G J, Byrne B J, Proc Natl Acad Sci USA. 1996 Nov. 26; 93(24):14082-7; and Curtis A. Machida. Methods in Molecular Medicine™. Viral Vectors for Gene Therapy Methods and Protocols. 10.1385/1-59259-304-6:201 © Humana Press Inc. 2003. Chapter 10. Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference).

Genebank reference numbers for sequences of AAV serotypes 1, 2, 3, 3B, 4, 5, 6, 7, 8, 9, 10, 11, 12, and 13 are listed in patent publication WO2012064960, which is incorporated herein by reference in its entirety.

A non-limiting method of rAAV particle production method is described next. One or more helper plasmids are produced or obtained, which comprise rep and cap ORFs for the desired AAV serotype and the adenoviral VA. E2A (DBP), and E4 genes under the transcriptional control of their native promoters. In some embodiments, the one or more helper plasmids comprise rep genes, cap genes, and optionally one or more of the adenoviral VA, E2A (DBP), and E4 genes under the transcriptional control of their native promoters. In some embodiments, the one or more helper plasmids comprise cap ORFs (and optionally rep ORFs) for the desired AAV serotype and the adenoviral VA, E2A (DBP), and E4 genes under the transcriptional control of their native promoters. The cap ORF may also comprise one or more modifications to produce a modified capsid protein as described herein. As an example, HEK293 cells (available from ATCC®) are transfected via CaPO4-mediated transfection, lipids or polymeric molecules such as Polyethylenimine (PEI) with the helper plasmid(s) and a plasmid containing a nucleic acid vector. The HEK293 cells are then incubated for at least 60 hours to allow for rAAV particle production. Alternatively, the HEK293 cells are transfected via methods described above with AAV-ITR containing one or more genes of interest, a helper plasmid comprising genes encoding Rep and Cap proteins, and co-infected with a helper virus. Helper viruses are viruses that allow the replication of AAV. Examples of helper virus are adenovirus and herpesvirus.

Alternatively, in another example, Sf9-based producer stable cell lines are infected with a single recombinant baculovirus containing the nucleic acid vector. As a further alternative, in another example HEK293 or BHK cell lines are infected with a HSV containing the nucleic acid vector and optionally one or more helper HSVs containing rep and cap ORFs as described herein and the adenoviral VA, E2A (DBP), and E4 genes under the transcriptional control of their native promoters. The HEK293, BHK, or Sf9 cells are then incubated for at least 60 hours to allow for rAAV particle production. The rAAV particles can then be purified using any method known in the art or described herein, e.g., by iodixanol step gradient, CsCl gradient, chromatography, or polyethylene glycol (PEG) precipitation.

Methods for large-scale production of AAV using a herpesvirus-based system are also known. See for example. Clement et al. (Hum Gene Ther. 2009, 20(8):796-806). Methods of producing exosome-associated AAV, which can be more resistant to neutralizing anti-AAV antibodies, are also known (Hudry et al., Gene Ther. 2016, 23(4):380-92; Macguire et al., Mol Ther. 2012, 20(5):960-71).

Methods for producing and using pseudotyped rAAV vectors are also known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

### Compositions

Various formulations have been developed to facilitate rAAV particle use. For example, for administration of an injectable aqueous solution of rAAV particles, the solution may be suitably buffered, if necessary, and the liquid diluent first rendered isotonic with sufficient saline or glucose. In some embodiments, a composition as provided herein comprises a plurality of any one of the rAAV particles disclosed herein. In some embodiments, a composition comprises pluralities of more than one of the rAAV particles disclosed herein. In some embodiments. “administering” or “administration” means providing a material to a subject in a manner that is pharmacologically useful.

Accordingly, in some embodiments, a composition of variant rAAV particles comprises a pharmaceutically acceptable carrier. The term “carrier” refers to a diluent, adjuvant, excipient, or vehicle with which the rAAV particle is administered. Such pharmaceutical carriers can be sterile liquids (e.g., water, oils, saline solutions, aqueous dextrose and glycerol solutions), suspending agents, preserving agents (e.g., methyl-, ethyl-, and propyl-hydroxy-benzoates), and pH adjusting agents (such as inorganic and organic acids and bases). In some embodiments, carriers include buffered saline solutions (e.g., phosphate buffered saline. HEPES-buffered saline). USP grade carriers and excipients are particularly useful for delivery of rAAV particles to human subjects. Such compositions may further optionally comprise a liposome, a lipid, a lipid complex, a microsphere, a microparticle, a nanosphere, or a nanoparticle, or may be otherwise formulated for administration to the cells, tissues, organs, or body of a subject in need thereof. Methods for making such compositions are well known and can be found in, for example, Remington: The Science and Practice of Pharmacy, 22nd edition, Pharmaceutical Press, 2012.

In some embodiments, a composition comprising any one of the rAAV particles disclosed herein comprises Balanced Salt Solution (BSS) supplemented with 0.014% Tween 20 (polysorbate 20).

Typically, compositions may contain at least about 0.1% of the therapeutic agent (e.g., rAAV particle) or more, although the percentage of the active ingredient(s) may be varied and may conveniently be between about 1 or 2% and about 70% or 80% or more of the weight or volume of the total formulation. Naturally, the amount of therapeutic agent(s) (e.g., rAAV particle) in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the an of preparing such pharmaceutical formulations, and as such, a variety of dosages and treatment regimens may be desirable.

The pharmaceutical forms of rAAV particle compositions suitable for injectable use include sterile aqueous solutions or dispersions. In some embodiments, the form is sterile and fluid to the extent that easy syringability exists. In some embodiments, the form is stable under the conditions of manufacture and storage and is preserved against the contaminating action of microorganisms, such as bacteria and fungi. In some embodiments, the form is sterile. The carrier can be a solvent or dispersion medium containing, for example, water, saline, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants.

Preparation of compositions for administration to a subject are known in the art. For example, a dosage may be dissolved in 1 ml of isotonic NaCl solution and either added to 1000 ml of hypodermoclysis fluid or injected at the proposed site of infusion. (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by, e.g., FDA Office of Biologics standards.

### Methods

In some aspects, the disclosure relates to methods for delivering an isolated nucleic acid or rAAV to a cell or cells (e.g., a cell or cells of a subject). In some aspects, the disclosure relates to the recognition that incorporation of a DD into certain fusion proteins, in some embodiments, prevents transit of the protein through cellular secretion pathways. The disclosure is based, in part, on the recognition that inclusion of a linker sequence between a sequence encoding a protein (e.g., a LIF protein) and a DD, in some embodiments, results in improved protein secretion.

Accordingly, in some embodiments the disclosure relates to methods of increasing secretion of a transgene product from a cell. In some embodiments, the method comprises introducing into a cell an isolated nucleic acid or rAAV as described by the disclosure (e.g., encoding a fusion protein comprising a protein coding sequence linked to a DD by a linker sequence), and an agent that binds to the DD. In some embodiments, the linker sequence comprises a furin cleavage sequence. In some embodiments, the agent is TMP.

The amount of increased secretion (e.g., increased secretion of the protein relative to secretion from a cell transfected with a sequence coding for the protein that does not include a linker sequence and/or DD) may vary. In some embodiments, secretion of the protein is increased 2-fold, 3-fold, 5-fold, 10-fold, 20-fold, 50-fold, 100-fold, 200-fold, 500-fold, 1000-fold, or more than 1000-fold.

Methods described by the disclosure are useful, in some embodiments, for the treatment of certain diseases or disorders (e.g., diseases and disorders of the eye, for example, retinal degeneration, retinitis pigmentosa (RP), age-related macular degeneration (AMD), glaucoma, etc.). In some embodiments, the disclosure provides a method for treating a disease or disorder in a subject in need thereof, the method comprising administering to the subject an rAAV encoding an isolated nucleic acid as described by the disclosure (e.g., an isolated nucleic acid encoding one or more of LIF, ASCL1, CNTF, EDN2, HBEGF, NRF2, OSM, OSM-M2, STAT3, STAT3 Y705F, STAT3 S727E, Cre recombinase, VEGFR2 IgG loops 1-3, VEGFR2 IgG loops 1-7, and VEGFR2 IgG loops 2-3 of VEGFR2.

As shown in the Examples, a neurotrophic factor-DD fusion protein (e.g., Retinal Protective Factor 2 (RPF2)) was produced. It was observed that, in some embodiments, TMP can be used to regulate RPF2 production in vitro and in vivo in a dose-dependent manner, and that expression was reversible by TMP withdrawal. It was also observed that, in some embodiments, long-term expression of RPF2 did not alter retinal function or morphology and rescued photoreceptors from an acute light-induced degeneration model as well as preserved cone vision in an rd10 inherited retinal degeneration model.

Accordingly, in some aspects, the disclosure provides a method for delivering a transgene to the eye of a subject, the method comprising administering an isolated nucleic acid as described herein, or rAAV as described herein, or composition as described herein, to the eye of a subject (e.g., a subject having or suspected of having a disease or disorder of the eye).

A subject is generally a mammal, for example a human, mouse, dog, cat, pig, horse, or non-human primate. In some embodiments, a subject is a human. In some embodiments, a subject in which a cell, tissue or organ is transduced is a vertebrate animal (e.g., a mammal or reptile). In some embodiments, a mammalian subject is a human, a non-human primate, a dog, a cat, a hamster, a mouse, a rat, a pig, a horse, a cow, a donkey or a rabbit. Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. In some embodiments, a subject is a model for a particular disease or used to study the pharmacokinetics and/or pharmacokinetics of a protein or siRNA encoded by a gene of interest.

A subject “having” a disease generally refers to a subject who exhibits one or more signs and/or symptoms of a particular disease. For example, a subject “having retinitis pigmentosa” may be a subject who exhibits one or more of the following signs and/or symptoms: trouble seeing at night, decreased peripheral vision, tunnel vision, photophobia, development of bone spicules in the fundus, blurring of vision, etc. In some embodiments, a subject “having” a disease has been diagnosed as having the disease by a medical professional (e.g., a medical doctor). In some embodiments, the diagnosis has been confirmed by a laboratory assay.

In some embodiments, a subject “suspected of having” a disease refers to a subject who is genetically predisposed (e.g., has one or more genetic factors, such as mutations) associated with increased risk of developing a particular disease. A subject “suspected of having” a disease may or may not exhibit one or more signs and/or symptoms of that disease. For example, a subject “suspected having retinitis pigmentosa” may be a subject who does not exhibit the following signs and/or symptoms—trouble seeing at night, decreased peripheral vision, tunnel vision, photophobia, development of bone spicules in the fundus, blurring of vision, etc.—but does have a mutation associated with development of RP in one of the following genes: RP1, RP2, RPGR, PRPH2, RP9, IMPDH1, PRPF31, CRB1, PRPF8, TULP1, CA4, HPRPF3, ABCA4, EYS, CERKL, FSCN2, TOPORS, SNRNP200, SEMA4A, PRCD, NR2E3, MERTK, USH2A, PROM1, KLHL7, CNGB1, BEST1, TTC8, C2Orf71, ARL6, ZFN516, DHDDS, LRAT, SPATA7, CRX, PAP1, etc.

In some embodiments, a subject “having” a disease has been diagnosed as having the disease by a medical professional (e.g., a medical doctor). In some embodiments, the diagnosis has been confirmed by a laboratory assay. In some embodiments, a subject “suspected of having” a disease has been diagnosed as having the disease by a medical professional but the diagnosis has not been confirmed by a laboratory assay.

To “treat” a disease as the term is used herein, means to reduce the frequency or severity of at least one sign or symptom of a disease or disorder experienced by a subject. The compositions described above or elsewhere herein are typically administered to a subject in an effective amount, that is, an amount capable of producing a desirable result. The desirable result will depend upon the active agent being administered. For example, an effective amount of rAAV particles may be an amount of the particles that are capable of transferring an expression construct to a host cell, tissue or organ. A therapeutically acceptable amount may be an amount that is capable of treating a disease, e.g., retinal degeneration or retinitis pigmentosa (RP). As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

In some embodiments, a specific tissue in the eye is targeted. For example, the retina or one or more cell type of the retina may be targeted (e.g., photoreceptors (PR), retinal ganglion cells (RGC), bipolar cells, retinal pigment epithelium (RPE) cells, amacrine cells, astrocytes, horizontal cell, microglia. Muller glia, etc.).

Some non-limiting examples of retinal diseases that may be treated using any one of the compositions provided herein include age-related macular degeneration, choroidermia, color blindness, Leber's congenital amaurosis, reitinitis pigmentosa, Stargardt's disease. Acromatopsia, Blue cone monochromacy. Cone-rod dystrophy, congenital stationary night-blindness. Leber's hereditary Optic Neuropathy. Glaucoma, and retinal degeneration (e.g., retinal degeneration due to light damage).

In some embodiments, a composition comprising any one or more of the variant rAAV particles disclosed herein is provided to photoreceptor cells (PRs). In some embodiments, a composition comprising any one or more of the rAAV particles disclosed herein is provided to retinal ganglion cells (RGCs). In some embodiments, a composition comprising rAAV particles is provided to a PR and/or RGC via an intravitreal injection to the subject carrying the PR and/or ROC. In some embodiments, a composition is provided via subretinal injection. Other non-limiting examples of routes to administrate a composition as disclosed herein to the eye include intracameral, periocular and subconjunctival injections. In some embodiments, a composition may be injected into the lateral geniculate nucleus of a subject. Such a method may be used to target RGCs. In some embodiments, a composition may be administered topically to an eye (e.g., in eye drops).

### EXAMPLES

### Example 1: Role of gp130 Ligands in Autosomal Retinitis Pigmentosa (RP)

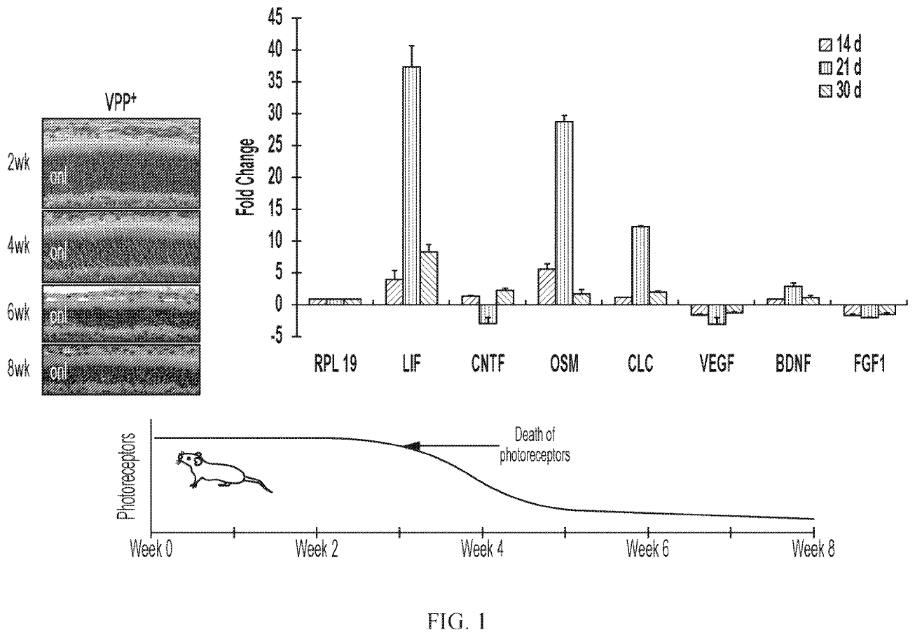

Gene expression in retinas with autosomal dominant retinitis pigmentosa (RP) was investigated in a VPP transgenic mouse model. FIG. 1 shows up-regulation of leukemia inhibitory factor (LIF). Oncostatin M (OSM), and Eosinophil lysophospholipase (CLC), which are all gp130 ligands. Other genes assayed included Receptor-like protein 19 (RLP19), ciliary neurotrophic factor (CNTF). Vascular endothelial growth factor (VEGF), Brain-derived neurotrophic factor (BDNF), and Heparin-binding growth factor 1 (FGF1).

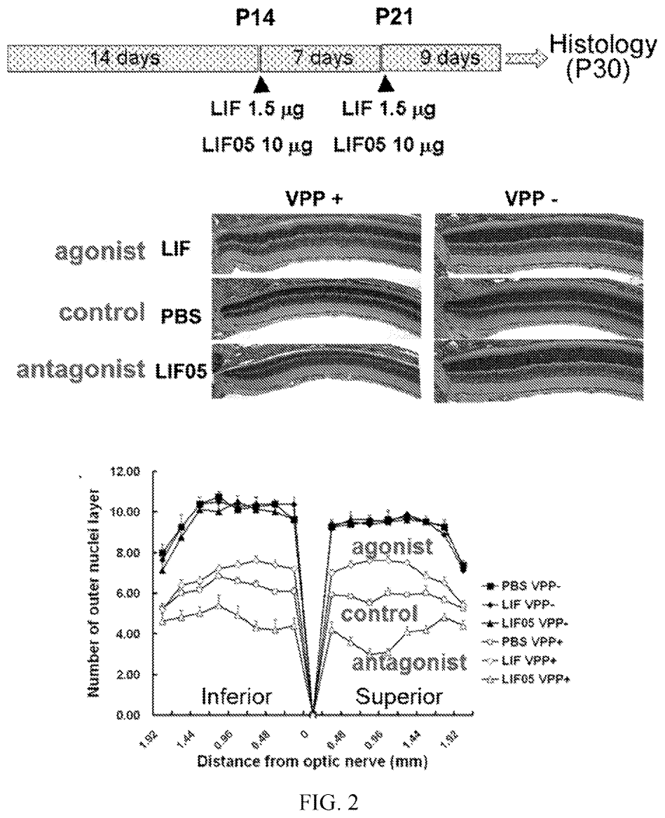

FIG. 2 shows injection of LIF delays photoreceptor degeneration in mice with autosomal dominant RP. On the contrary, injection of LIF05, an antagonist of LIF receptor, accelerates degeneration.

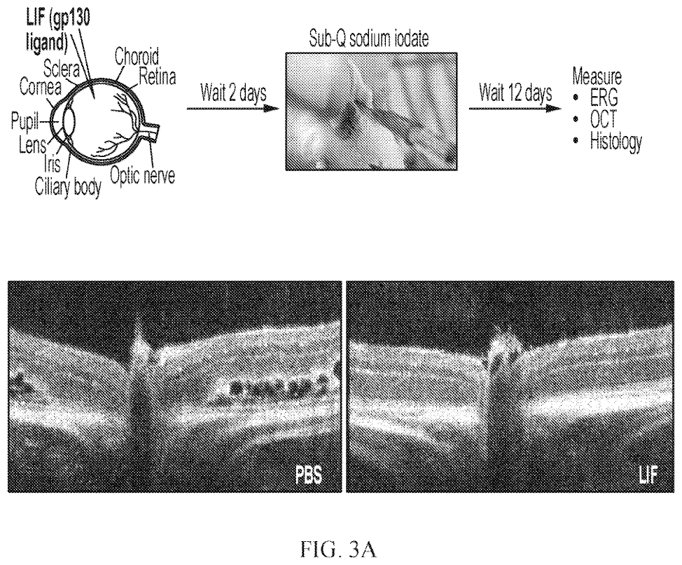

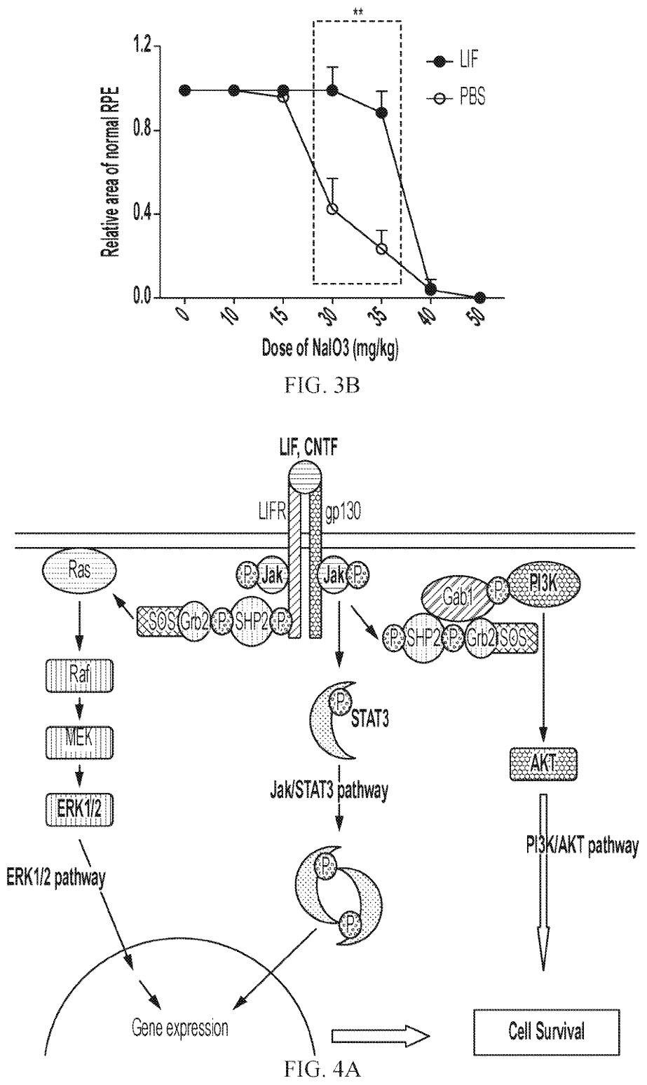

FIG. 3A shows LIF injection protects retinal pigment epithelium (RPE) from degeneration caused by sodium iodate (NAIO₃) injection. FIG. 3B shows data indicating that LIF injection increases RPE resistance to NAIO₃.

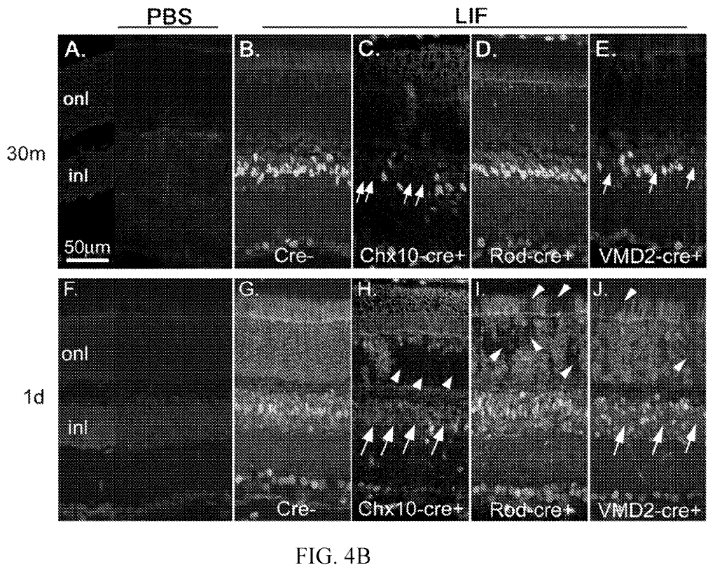

FIG. 4A shows a schematic depicting signaling pathways associated with LIF/CNTF receptors. FIG. 4B shows microscopy data indicating loss of LIF-induced phosphorylated STAT3 (pSTST3) in gp130 targeted mice.

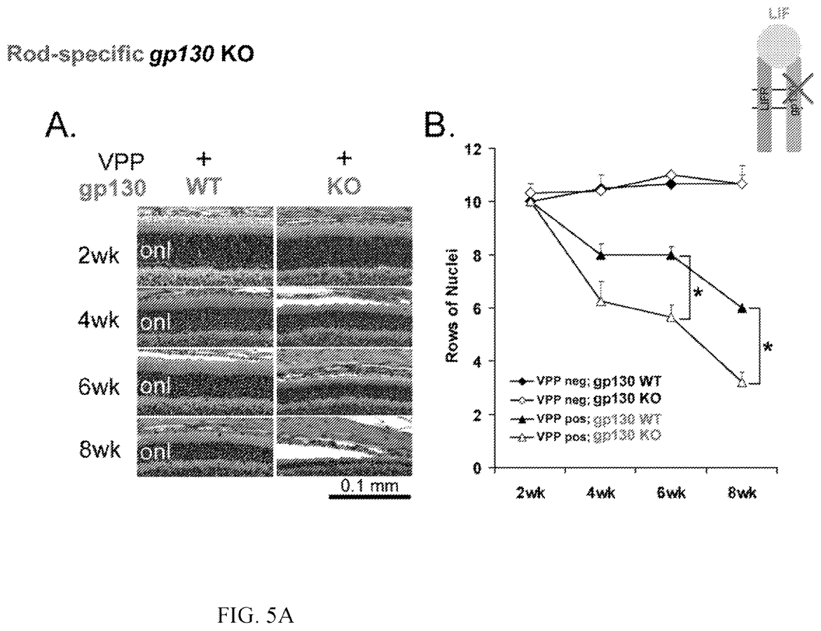

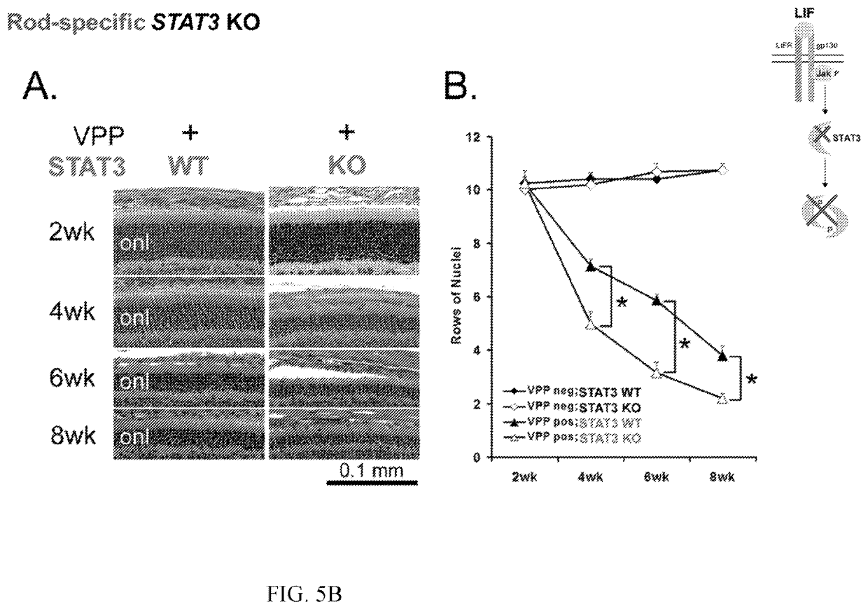

FIG. 5A shows loss of gp130 in rod photoreceptors accelerates photoreceptor cell death in a genetic model of retinal degeneration. FIG. 5B shows loss of STAT3 in rod photoreceptors accelerates photoreceptor cell death in a genetic model of retinal degeneration.

In summary, the data described in this example indicate the following regarding protection induced by LIF:
1. In some embodiments, cytokines protect photoreceptors from LD and inherited mutations (Opsin^{VPP} and PDE6B^{RD10}).
2. In some embodiments, cytokines protect RPE from sodium iodate.
3. In some embodiments. LIF-induced protection requires GP130 and STAT3 expression in protected cells.
4. In some embodiments, part of LIF-induced protection is mediated by downregulation of phototransduction in PR and the visual cycle in RPE.
5. In some embodiments, part of LIF-induced protection is mediated by protecting mitochondria.
6. In some embodiments, STAT3 regulates NRF2 pathway including oxidative defense. DNA repair, and increasing proteostasis.

### Example 2: LIF Expression Vectors

### Materials and Methods

### Mice

BALB/cJ, C57BL/6, and rd10 breeders were obtained and colonies were established. All mice were reared in 12 hour cyclic dim light (<100 lux))/dark conditions (6 AM-6 PM) in the University of Florida animal housing facility and provided water and food ad libitum.

### Vector Production

The AAV vector plasmid, ‘Sc-smCBA-mCherry’, contains a chimeric CMV/chicken beta-actin promoter driving expression of the mCherry cDNA. The AAV terminal repeat (ITR) contained a mutated terminal resolution site to enable packaging of a self-complementary vector genome. The AAV vector plasmid, ‘pTR-UF11’ contains the full chimeric CMV/chicken beta-actin promoter (CBA) driving green fluorescent protein (GFP) cDNA. The AAV vector plasmid ‘pTR-CBA-hLIF’ contains the CBA promoter driving human LIF cDNA. The AAV vector plasmid ‘pTR-CBA-RPF2’ contains the CBA promoter driving the RPF2 cDNA. The RPF2 cDNA was created by de-novo synthesis from Genscript (Piscataway, NJ). For packaging of AAV6 (trp Mut) an AAV2 rep-AAV6 cap plasmid containing substitutions resulting in a tyrosine to phenylalanine substitution at amino acid position 705 and 731 (Y705+731F) and threonine to valine substitution at position 492 (T492V). The rep-cap plasmid contains AAV2 cap with Y272F, Y444F, Y500F, Y730F and T491V mutations. Vector plasmids, AAV rep-cap and Ad helper plasmid were co-transfected into HEK293T cells and the virus was harvested and purified. Purified viruses were resuspended in balanced sail solution (BSS) (Alcon, Ft. Worth TX) supplemented with Tween-20 (0.014%) and stored at −80° C. until use. All viruses were titered by qPCR and tested for endotoxin.

### Cell Culture Transfections, Trimethoprim Treatment, and Conditioned Media Experiments

The rat miller glial-like cell line (rMC-1) was used in this example. The cells were cultured in high glucose Dulbecco's minimal essential medium (DMEM) supplemented with 10% fetal bovine serum 100 U/mL penicillin, and 100 μg/mL streptomycin and 1× GlutaMax (Thermo Fisher Scientific Inc., Waltham, MA). At 80% confluence, the cells were transfected with a purified plasmid using the standard Lipofectamine 3000 protocol (Thermo Fisher Scientific Inc., Waltham, MA). 24 hours after transfection, the media was replaced with serum-free DMEM. The cells were then dosed overnight with varying concentrations of trimethoprim or control vehicle (20% DMSO). The conditioned media was then collected for ELISA or STAT3 activation studies. For conditioned media experiments, rMC-1 cells were grown in 6-well plates. The serum-deprived cells were incubated for 30 minutes with conditioned media collected from transfected rMC-1 cells treated with trimethoprim. The cells were then washed with cold PBS and harvested for analysis by immunoblotting.

### Immunoblotting

Retinal tissue or rMC-1 cells were homogenized in RIPA buffer (Sigma-Aldrich, St. Louis. MO) containing a protease inhibitor cocktail (Merck Millipore. Billerica. MA) supplemented with sodium fluoride and sodium orthovanadate (New England BioLabs Inc., Ipswich, MA). Protein lysate concentration was quantified by BCA assay (Thermo Fisher Scientific Inc., Waltham, MA) before boiling and subjecting to SDS-PAGE. Lysates were run on 4%-20 k polyacrylamide gel (Thermo Fisher Scientific Inc., Waltham, MA) and transferred onto polyvinylidene fluoride membranes (Merck Millipore, Billerica, MA). Membranes were blocked with Odyssey blocking buffer (LI-COR Biosciences. Lincoln, NE) and incubated overnight with primary antibodies. Secondary antibodies conjugated with IRDyes were used. Signals were detected using the Odyssey CLx Imaging System (LI-COR Biosciences. Lincoln, NE). Intensities of the protein bands were quantified using Image Studio 5 (LI-COR Biosciences. Lincoln, NE).

### Enzyme-Linked Immunosorbent Assay (ELISA)

LIF and RPF-2 levels were detected in retinal tissue lysates and rMC-1 conditioned media. For tissue lysates, the retinas were prepared in an ELISA lysis buffer (1% IGEPAL—CA 630, 135 mM NaCl, 20 mM Tris and 2 mM EDTA). Lysates or conditioned media was incubated for 2 hours on plates containing pre-blocked capture antibodies followed by 1-hour incubations with biotinylated detection antibody and streptavidin conjugated to horseradish peroxidase. Signals were developed using the Substrate Reagent Pack (R&D Systems, Inc., Minneapolis, MN) and read at 450 nm wavelength using the CLARIOstar microplate reader (BMG LABTECH GmbH. Ortenberg, Germany).

### Trimethoprim (TMP) Delivery

For the in vivo dose response, varying concentrations of TMP was resuspended in a solution of 20% DMSO. The mice were intraperitoneally injected once per day for seven days with equal volumes of the varying TMP dosage or vehicle. For the reversal study, mice were injected once per day for seven days followed by no administration of the drug for a subsequent seven days before the retinas were harvested for ELISA analysis. For the TMP food supplement, 600 ppm of trimethoprim was added to the Teklad LM-485 mouse diet (Envigo, Huntingdon. United Kingdom). The TMP food was provided ad libitum to the mice.

### Intravitreal Injections

Mice were anesthetized with ketamine (70-100 mg/kg) and xylazine (5-10 mg/kg). The eyes were dilated with a single drop of tropicamide and phenylephrine (Alcon, Ft. Worth TX). 1 μl of viral vector or Balanced Saline Solution (Alcon, Ft. Worth TX) were separately injected into the vitreous through the temporal limbus of the eye using a syringe with a 33-gauge needle (Hamilton Company, Reno. NV) AK-Poly-Bac ophthalmic medication (Alcon, Ft. Worth TX) was used to prevent post-surgical infections and suppress inflammation. Eyes were evaluated by OCT one week post-injection, and any animals with unresolved surgical trauma were excluded from the study.

### Electroretinography (ERG)

Mice were anesthetized with ketamine (70-100 mg/kg) and xylazine (5-10 mg/kg). The eyes were dilated with a single drop of tropicamide and phenylephrine (Alcon. Ft. Worth TX). Retinal function was measured using the Colordome ERG system (Diagnosys, Dorset. United Kingdom). To record electrical responses from both eyes, gold wire electrodes were placed on the corneas, the platinum reference electrode was placed in the mouth and the platinum ground electrode was attached to the tail. The body temperature was maintained at 37° C. throughout the experiment using the built-in heating pad of the ERG system. For scotopic ERGs, the animals were dark adapted for at least 0.12 hours. A series of increasing light flash intensities ranging from 0.0001-180 cd·s/m² were used. The a-wave was determined as the trough of the negative deflection from the baseline while the b-wave was measured from the a-wave to the peak of the positive deflection. For photopic ERGs, to prevent rod photoreceptor contribution to the recording, a background illumination of 30 cd·s/m² was used to bleach the rods. A series of increasing light flash intensities above the background illumination (photopic 1.875-60 cd·s/m²) were used to generate a cone response.

### Spectral Domain-Optical Coherence Tomography (OCT) and Scoring

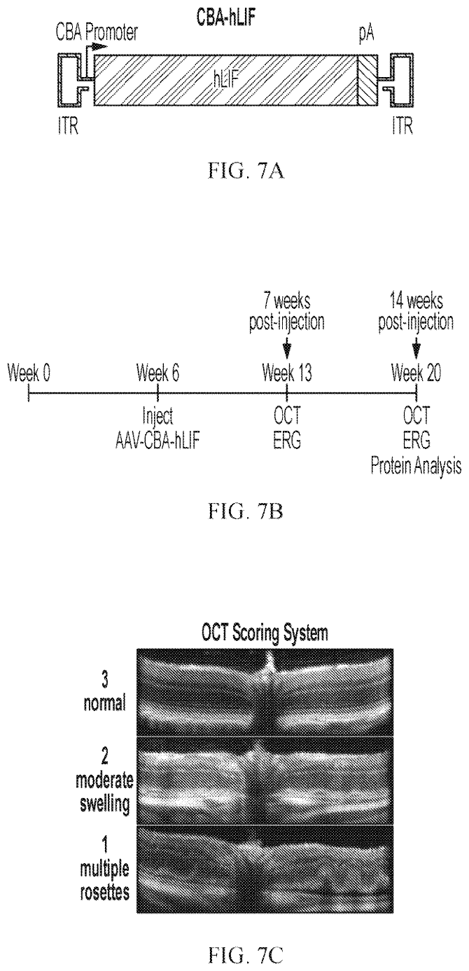

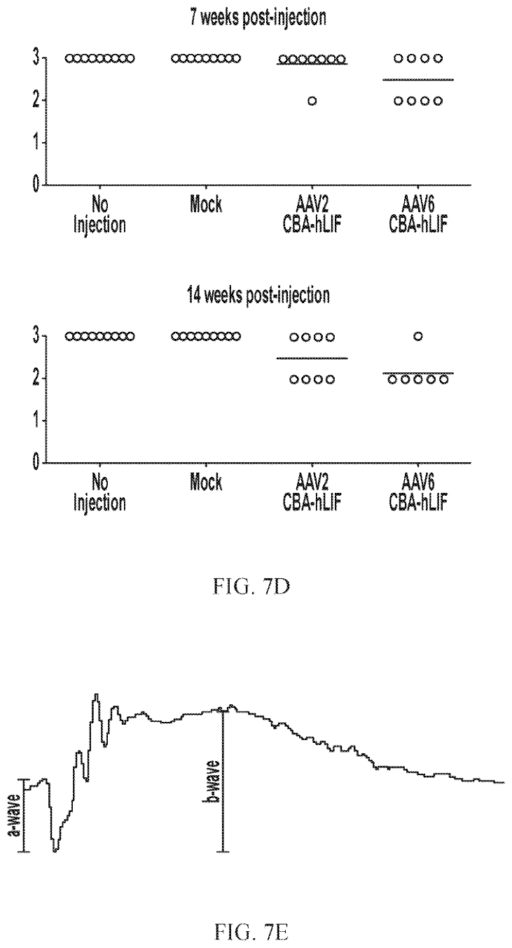

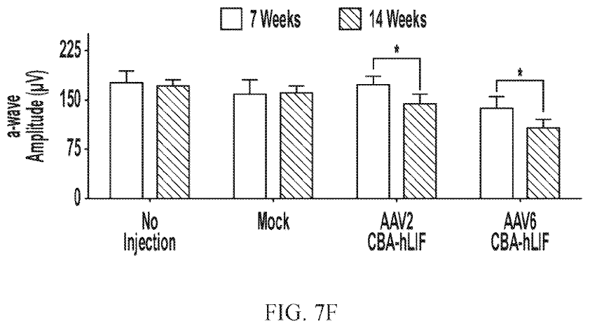

For in vivo retinal imaging. Spectral Domain-OCT images were obtained using the Envisu C-Class OCT system (Leica Microsystems. Wetzlar, Germany) Mice were first anesthetized with ketamine (70-100 mg/kg) and xylazine (5-10 mg/kg), followed by dilation with a single drop of tropicamide and phenylephrine (Alcon. Ft. Worth TX). Cornea clarity was maintained using GenTeal lubricating eye gel (Novartis Pharmaceuticals Corp., Basil. Switzerland). The mice were placed on a custom-built stage secured with a bite bar to allow free rotation for imaging. The stage was adjusted manually to center the image of the retina at the optic nerve head. Cross-sectional images were generated using 425 rectangular volume scans, which were averaged every five scans prior to image analysis. Images were acquired for both eyes. Averaged OCT images were analyzed using InVivoVue 2.4 (Leica Microsystems, Wetzlar, Germany). Outer nuclear layer (ONL) thickness was measured using the linear caliper function in the software by a masked observer using a pre-established uniform grid. The OCT images were also graded utilizing a scoring system ranging from 1 to 3 by a masked observer (FIG. 7C). A score of 3 was used to indicate a healthy-normal retina with no thinning and little to no inflammation. A scoring of 2 indicated a retina with slight edema, minor thinning, or inflammation. A scoring of 1 signified a degenerated retina with severe thinning, severe inflammation, missing external limiting membrane, or visible retinal detachment.

### Immunohistochemistry

To obtain retinal tissue sections, mouse eyes were enucleated with forceps and placed in cold PBS. Under a dissecting microscope, the cornea and lens were dissected away and the remaining eye cup was processed for cryosectioning. In additional eyes, retinal flat-mounts were made by gently dissecting the retina from the eyecup. Four incisions were made from the periphery to the center of the retina, to flatten the retina unto a microscope slide. Eyecups and flat mounts were fixed in 4% paraformaldehyde for 30 minutes at room temperature. The fixed eyecups were cryopreserved in 30% sucrose in PBS overnight at 4° C. The sample, were embedded in Tissue-Tek OCT Compound (Sakura Fintek USA Inc., Torrance, CA) and immediately frozen in liquid nitrogen. Tissue sections were cut at a thickness of 16 μm on a cryostat and placed onto glass slides. Slides were stored at −20° C. until needed for immunostaining. For IHC, the flat mount retinas or eyecup sectioned slides were washed with PBS containing 1% Triton X-100 (PBS-T). To prevent nonspecific binding, the tissue was blocked with 10% horse serum in PBS-T for at least 1 hour at room temperature. Primary antibody was applied to each sample for incubation overnight at 4° C. Samples were washed with PBS-T before incubating with secondary antibody for 1 hour at room temperature. Nuclei were counter-stained with 4-6-Diamidino-2-phenylindole (DAPI) before mounting on glass coverslip with 60% glycerol. Imaging was performed using the BZ-9000 fluorescence microscope (Keyence Corp., Osaka, Japan). The exposure time of each fluorescent channel was kept constant between samples for a given antibody. For flat mount counting of cone outer segments, a 250 mm² grid was superimposed over five different areas of each flat mount image. Cells within the grid were counted. All data collection was done by a masked observer.

### Light Damage

Mice 2-3 months old were exposed to varying intensities of light for a set amount of time. For light experiments, mice are placed (2 mice per cage) in a cage with a lid equipped with LED lights (white light). Light intensity can be adjusted using a connected dimmer switch, and a timer is set for the appropriate amount of time for each experiment. A light meter is used to measure the desired light intensity inside the cage. The mice are kept in a ventilated rack for the entire length of the experiment. At the end of the experiment, mice cages are replaced with a normal cage lid and moved back into dim light conditions for a one week wait until downstream structural and functional analysis.

### OptoMotry

To test the visual acuity of mice the OptoMotry system was used. The system consists of 4 computer screens that display alternating vertical light and dark bar that appear as a virtual rotating 3D cylinder. The mouse is placed on a non-rotating pedestal in the middle of the enclosure. The 3D cylinder pattern was rotated at a fixed speed of 12 degrees per second and the contrast was maintained at 100%. Head tracking was monitored using an overhead camera, and recorded by a masked observer. The staircase method was used to determine visual acuity by establishing the threshold of spatial frequency that the animal can track. An animal was considered to be tracking if its head followed the direction of the stimulus with a speed similar to the pattern movement.

### Fundus Imaging

For fundus imaging, a Micron III camera (Phoenix Research Labs. Pleasanton, CA) was used. Mice were anesthetized with ketamine (70-100 mg/kg) and xylazine (5-10 mg/kg) and dilated with a single drop of tropicamide and phenylephrine (Alcon. Ft. Worth TX). Corneal clarity was maintained using GenTeal lubricating eye gel (Novartis Pharmaceuticals Corp., Basil. Switzerland). The mice were stabilized on a custom holder that allowed adjustments to center the image of the retina at the optic nerve head. Imaging was performed in under brightfield illumination, and using filtered red, and green channels. Images were acquired for both eyes.

### Statistical Analysis

Statistical analysis was carried out using Prism 6 (GraphPad Software Inc., La Jolla. CA). Statistical significance between multiple groups was determined by using Analysis of Variance (ANOVA) followed by Holm-Sidak post hoc test. p-value <0.05 was considered to be significant.

Unregulated. Long-Term Cytokine Expression is Detrimental to the Retina

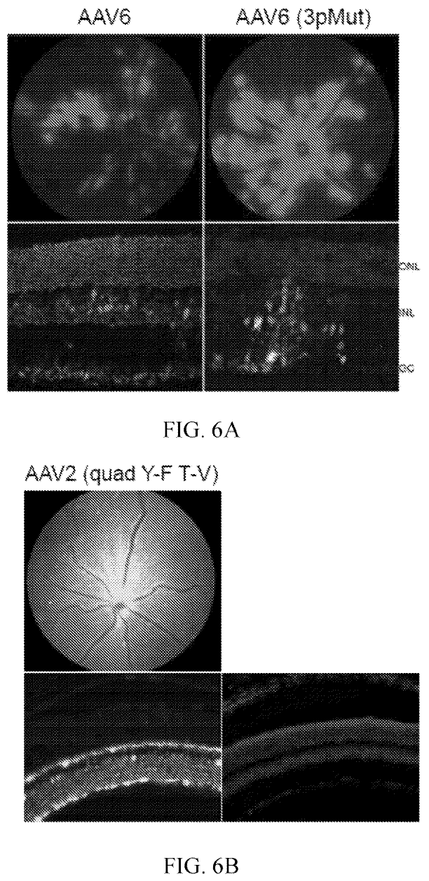

Two AAV serotypes were used to deliver the human LIF (hLIF) transgene to the retina by intravitreal injection, each with a unique tropism for retinal cells. AAV2 quadYF+TV (referred to as AAV2*) was selected since it can efficiently transduce a broad range of cells types in the murine retina by intravitreal injections. A triple mutant AAV6 serotype (referred to as AAV6*) was also investigated. This mutant serotype, in some embodiments, has increased transduction of cells in the inner nuclear layer, particularly Müller glial cells, compared to the wild-type AAV6 serotype via intravitreal delivery (FIGS. 6A-6B).

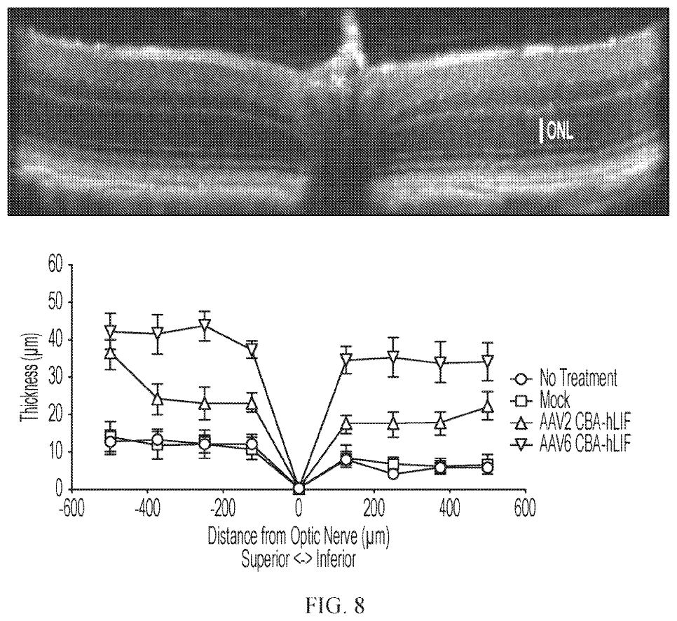

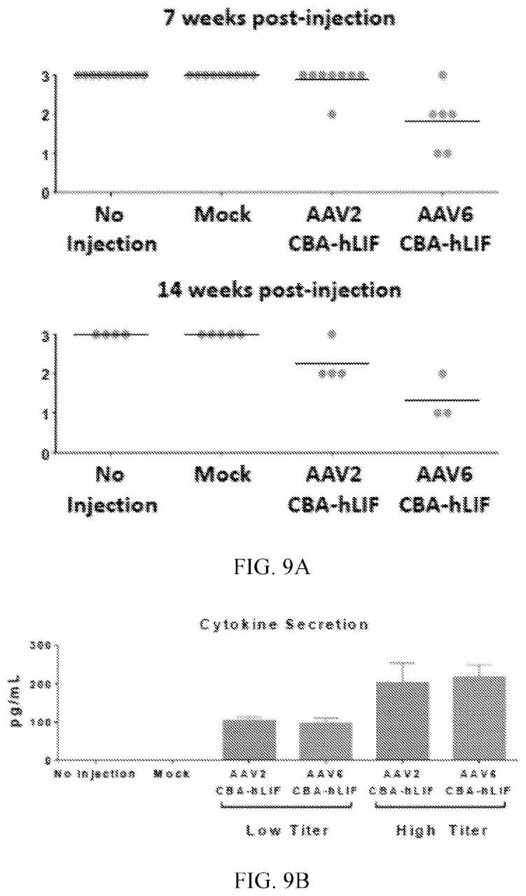

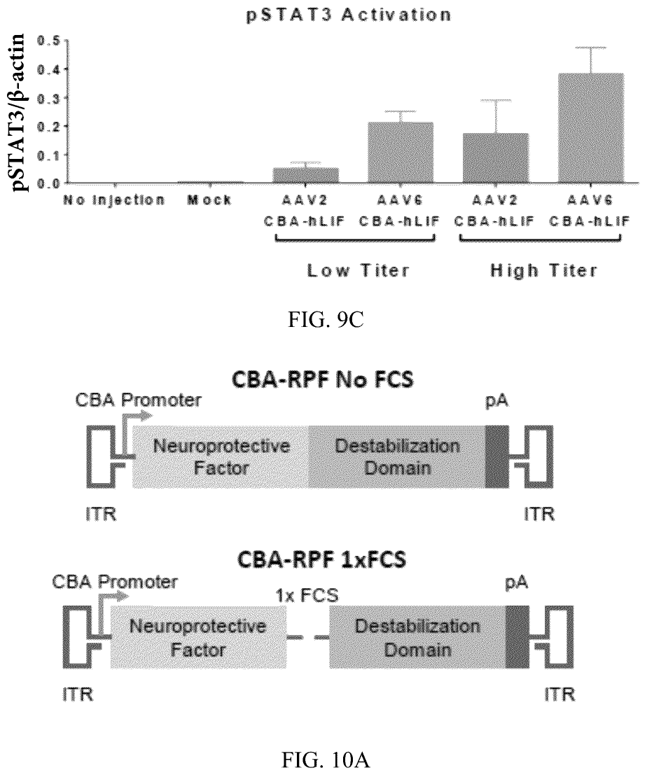

AAV2* and AAV6* vectors expressing the human leukemia inhibitory factor (hLIF) transgene under the control of the chicken beta-actin promoter (CBA) were injected into the vitreous of Balb/cJ mice at two titers (1×10⁸ viral genomes/μL for low titer, 1×10⁹ viral genomes/μL for high titer) (FIG. 7A). After seven weeks, mice were exposed to damaging bright light that was calibrated to synchronously kill approximately 50% of photoreceptors in a well-established light damage model (LD). Mice injected with either serotype of AAV-hLIF had significant preservation of photoreceptors in the outer nuclear (ONL) compared to controls (FIG. 8). However, despite this protective activity, long-term studies on treated mice not exposed to damaging light had progressive abnormal retinal structures consistent with edema, as seen by optical coherence tomography (OCT) imaging (FIGS. 7B-7D). Long-term expression also caused a reduction in photoreceptor light responses as measured by electroretinography (ERG) (FIGS. 7E-7F). The edema in AAV6*-treated mice was slightly worse than in AAV2*-treated mice at low titers (FIGS. 7D-7F). At high titers, the edema was significantly worse in both serotype groups. (FIG. 9A). The severity of adverse retinal changes (edema and loss of function) correlated with the level of LIF protein expression and activation of STAT3 (FIGS. 9B-9C). Overall, these results indicate that unregulated, high LIF expression is detrimental to the retina.

### Retinal Protective Factor 2

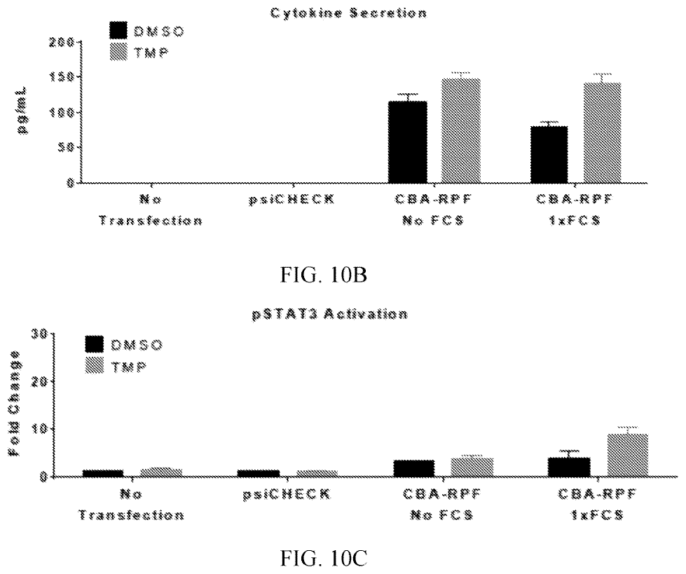

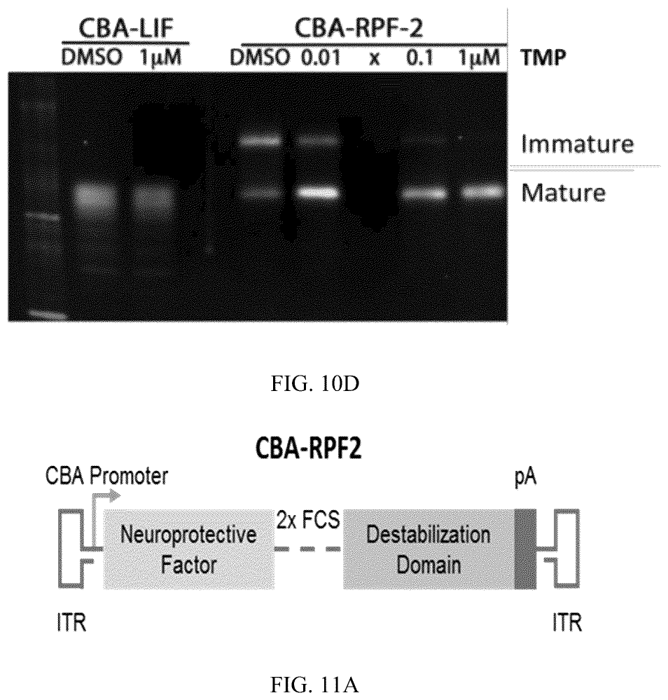

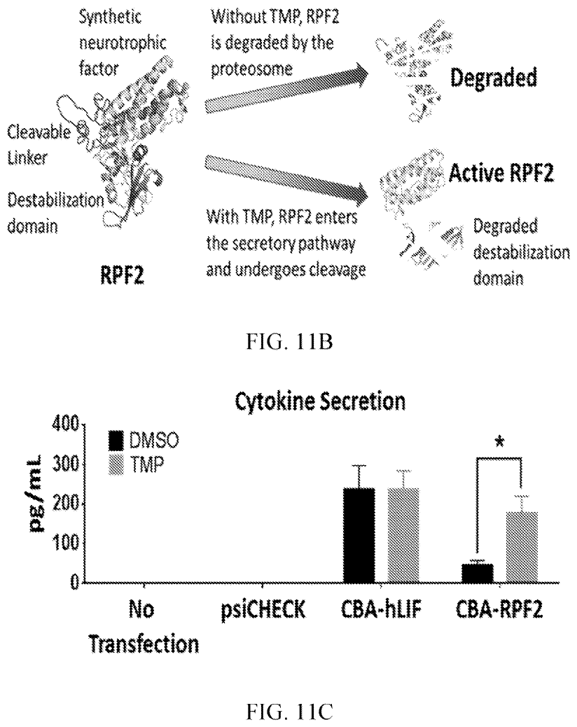

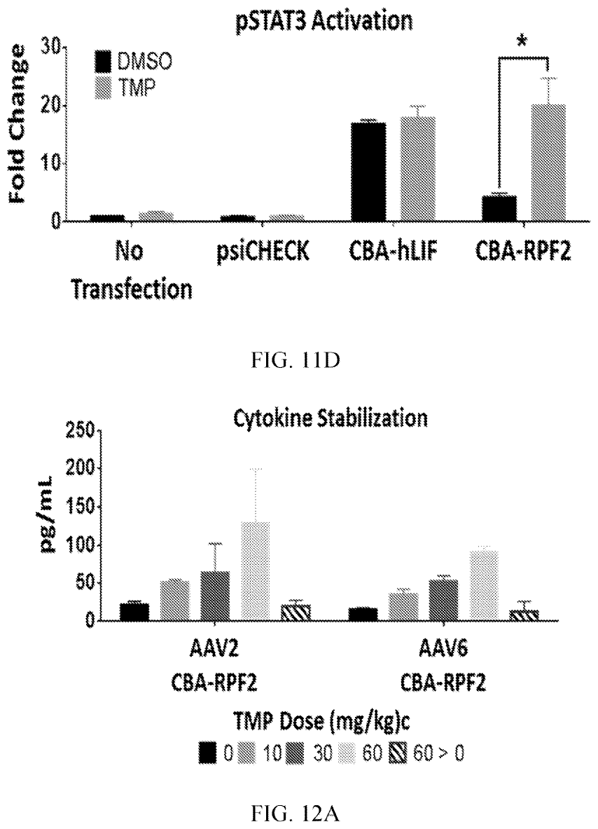

This example describes a synthetic and codon optimized E. coli dihydrofolate reductase cDNA (e.g., a destabilization domain. “DD”) fused to an hLIF cDNA (FIG. 10A). While the resulting fusion protein was expressed in cell culture, it was not regulated by TMP and was unable to activate STAT3 downstream of its receptor (FIG. 10C), likely because each domain in the fusion protein inhibited the activity of the other. Sequences encoding an eight amino acid linker with one furin cleavage site (FCS) were added between LIF and DD, or sequences encoding a twelve amino acid linker with two FCS (FIG. 10; FIG. 11A). This strategy takes advantage of the furin protease system which will cleave the domains as they transit the secretory pathway (FIG. 11B). The fusion proteins were tested in vitro, and the 12 amino acid linker containing two FCS produced the highest levels of cleaved cytokine with TMP treatment and had minimally detectable levels of cytokine in the absence of TMP (FIGS. 11C; 10B and 10D). In addition, the expression of cleaved and secreted cytokine was regulated by TMP in a dose-dependent manner (FIG. 10). The effects of TMP required the DD fusion since TMP did not alter the expression of cytokine in the hLIF-transfected group.

To determine whether the secreted RPF2 was functional, the conditioned media from transfected cells were collected and used to stimulate STAT3 in untransfected rMC-1 cells. Only conditioned media from TMP treated cells led to a significant increase in STAT3 activation (FIG. 11D). These levels were comparable to that of the hLIF-transduced group. In vitro data indicate that RPF2 is regulated in a dose-dependent manner and is secreted in its functional form to activate STAT3. The fusion protein with two FCS was used in AAV vector development and was named Retina Protective Factor 2(RPF2).

### AAV Expression of RPF2 is Regulated by IMP in a Dose-Dependent Manner In Vivo

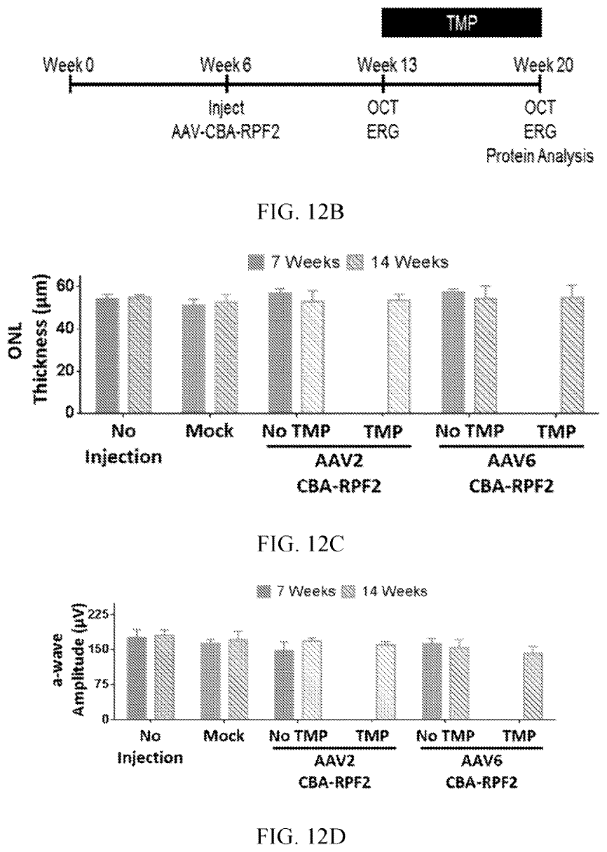

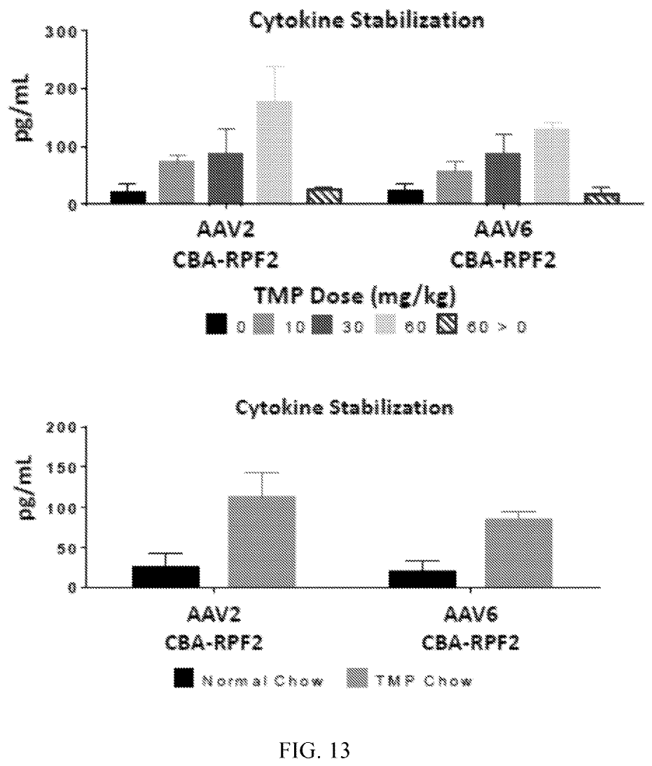

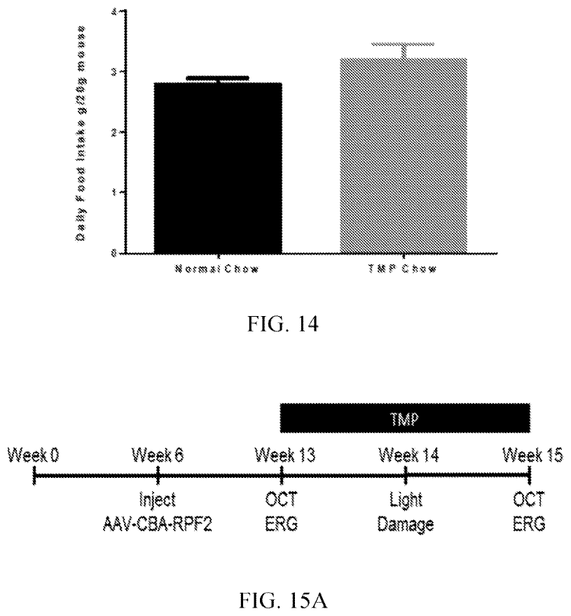

RPF2 was packaged into both AAV2* and AAV6* serotypes and intravitreally injected into Balb/cJ mice at high and low titers. Six weeks after AAV injections, mice were given six daily IP injections of the indicated doses of TMP. RPF2 levels rose dramatically with increasing doses of TMP and fell to baseline following a 7 day TMP withdrawal (FIG. 12A). Cytokine levels for both the 0 mg/kg dose and TMP withdrawal groups were below the limit of detection for the assay. The expressed cytokine was also functional since STAT3 activation in the retina was higher in animals treated with TMP. To characterize the long-term effects of the AAV-RPF2 therapies, retinal structure and function were monitored over time following viral delivery (FIG. 12B). Animals were placed on chow containing TMP starting 7 weeks post-injection. This route of drug administration yielded RPF2 stabilization at levels comparable to that of the 60 mg/kg systemic TMP delivery and did not affect the animal's daily food intake (FIGS. 13 and 14). At the highest dose of TMP, the cytokine levels were comparable but lower than the AAV-hLIF injected group (FIG. 8). After 7 weeks of TMP treatment, mice given AAV-RPF2 had normal retinal morphology and clearly lacked the edema observed in the AAV-hLIF treated mice (FIG. 12C; FIG. 13). Mice also had normal photoreceptor function as reflected in scotopic ERG responses (FIG. 12D). These data indicate that AAV-RPF2 therapies are regulated by TMP in a dose-dependent manner in vivo, and long-term therapy did not result in the negative outcomes observed using high expression of AAV-hLIF.

AAV-Expressed RPF2 can Protect Photoreceptors from Acute Light Injury

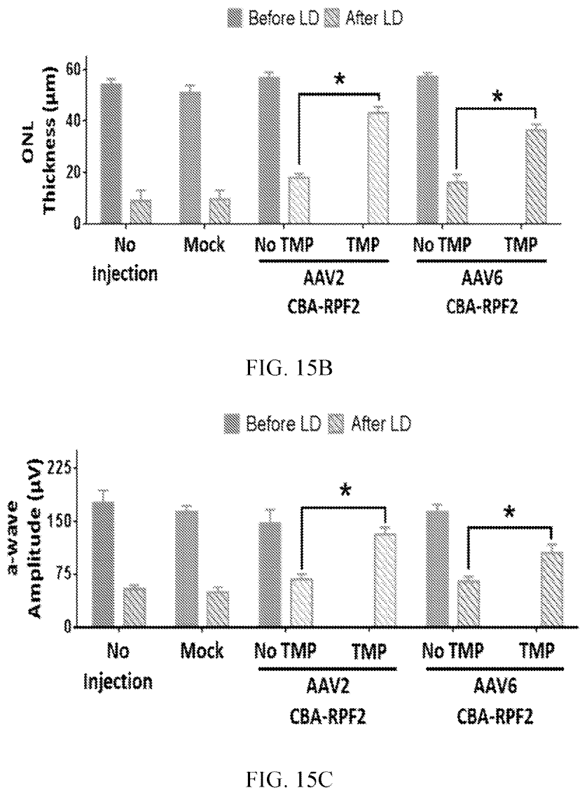

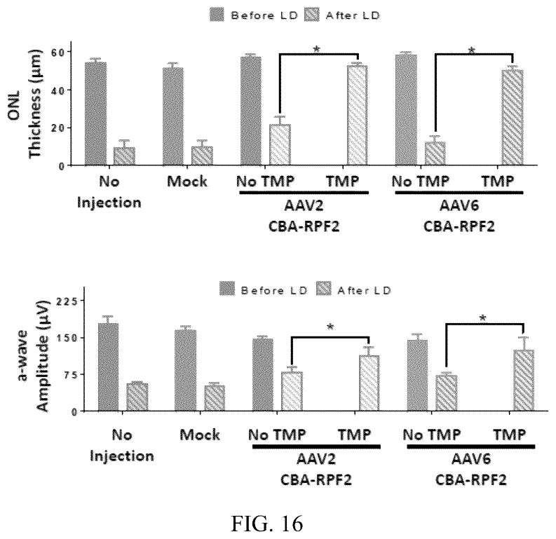

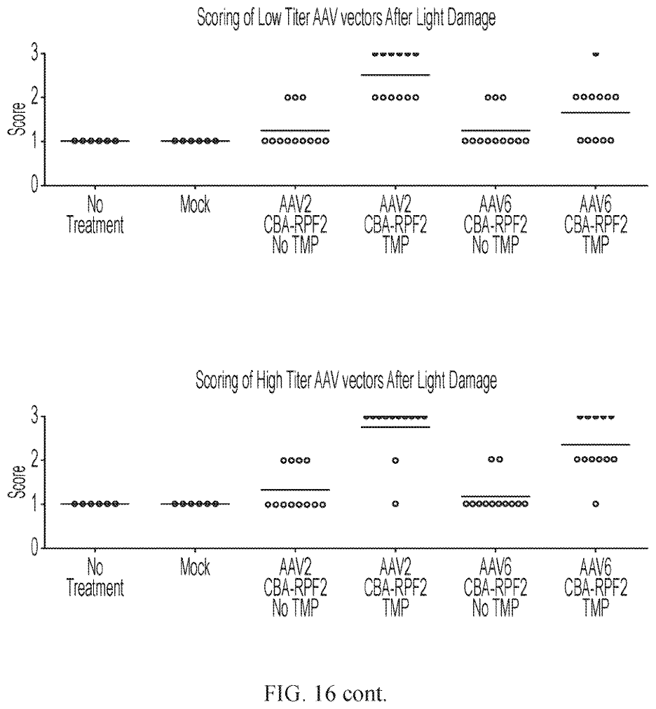

To determine whether AAV-RPF2 therapy can protect photoreceptors from light damage (LD), six-week-old mice were injected with AAV-RPF2 containing virus at low and high titers. After 7 weeks, mice were given TMP in mouse chow for 7 days prior to exposure to damaging bright light (FIG. 15A). In the presence of TMP, animals injected with either AAV-RPF2 serotypes showed significant preservation of ONL thickness compared to mock-injected or no TMP control groups, indicating that TMP-induced cytokine protected photoreceptors (FIG. 15B; FIG. 16). TMP induced RPF2 not only preserved structure but also preserved photoreceptor function as well (FIG. 15C; FIG. 16). High-titer AAV-RPF2 groups had greater preservation of the ONL than the low titer groups (FIG. 16).

### AAV-RPF2 Protects Photoreceptors in a Model of Inherited Retinal Degeneration

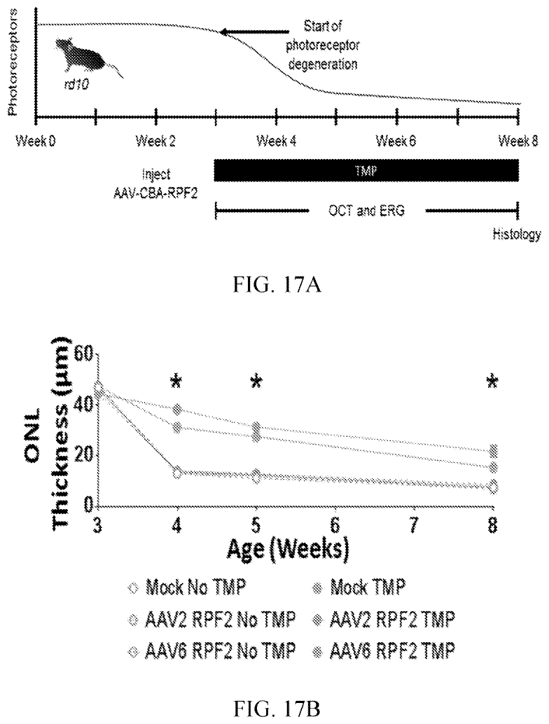

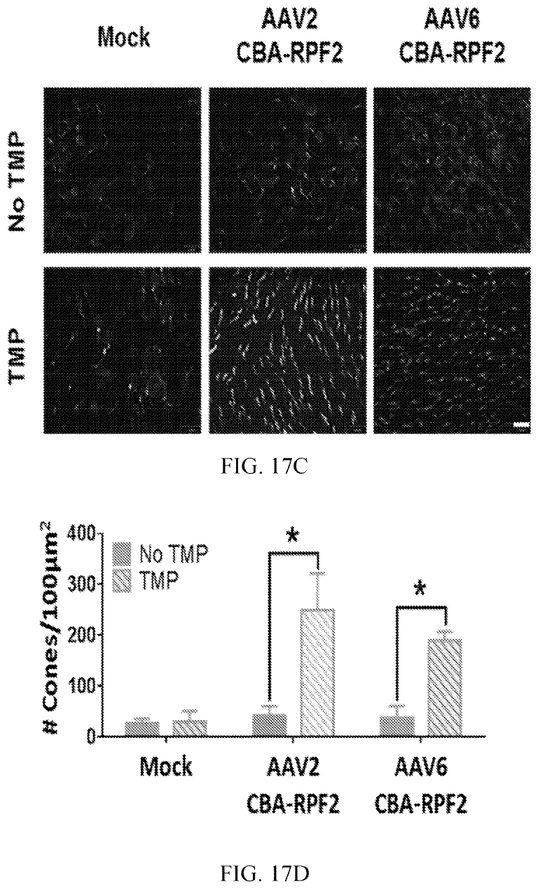

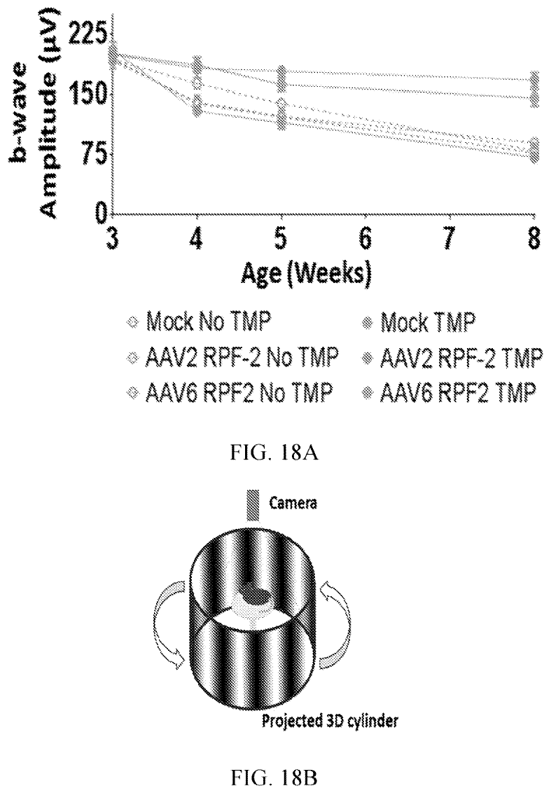

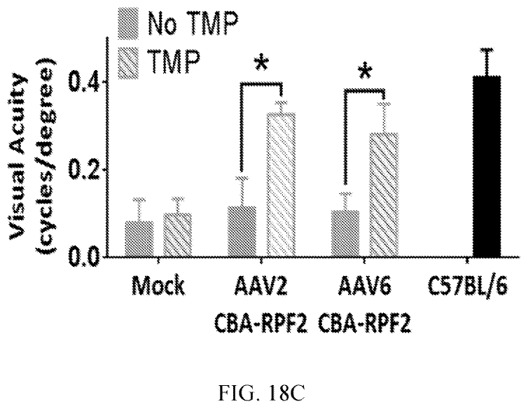

The rd10 mouse is a widely used model to study inherited retinal degeneration. To determine whether TMP-induced RPF2 expression can preserve photoreceptors in rd10 retinas. AAV-RPF2 vectors were delivered at high titer to the animals prior to any degeneration at week 2 (FIG. 17A). At week 3 (weaning age). TMP was administered and the animals were monitored by OCT and ERG over time (FIG. 17A). The TMP-treated AAV-RPF2 groups had significant preservation of the ONL compared to no TMP controls (FIG. 17B). Retinas were also collected at week 8 to measure preservation of cones, which normally die subsequent to rod loss. In the AAV-RPF2 groups treated with TMP, cones were markedly preserved compared to mock-infected mice or no TMP controls (FIGS. 17C-17D). The preserved cones maintained 80% of their function in the TMP-treated AAV-RPF2 groups, as measured by photopic ERG responses (FIG. 18A). To quantify functional vision in treated mice. OptoMotry was used to measure visual acuity (FIG. 18B). AAV-RPF2 groups treated with TMP showed significant preservation of visual acuity compared to mock-injected or no-TMP groups. The visual acuity in IMP treated mice was comparable to that of C57BL/6J wild-type control mice (FIG. 18C). Overall, data indicate AAV-RPF2 therapy protects rods and cones and preserves cone vision in an aggressive model of inherited retinal degeneration.

### Example 3: Additional Expression Vectors

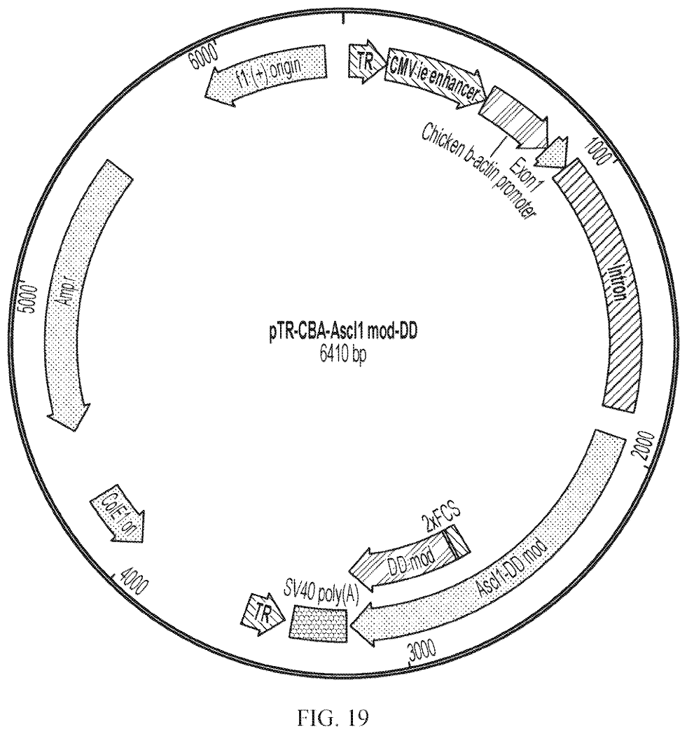

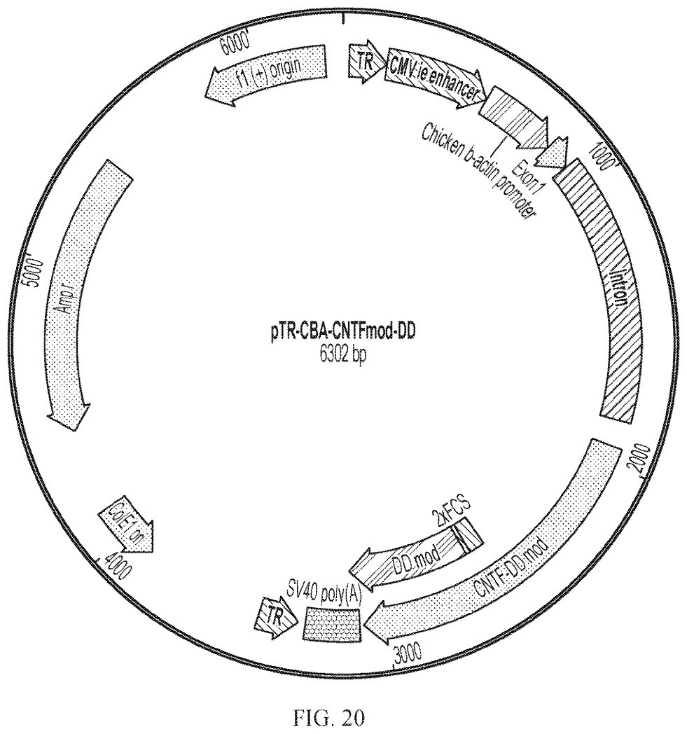

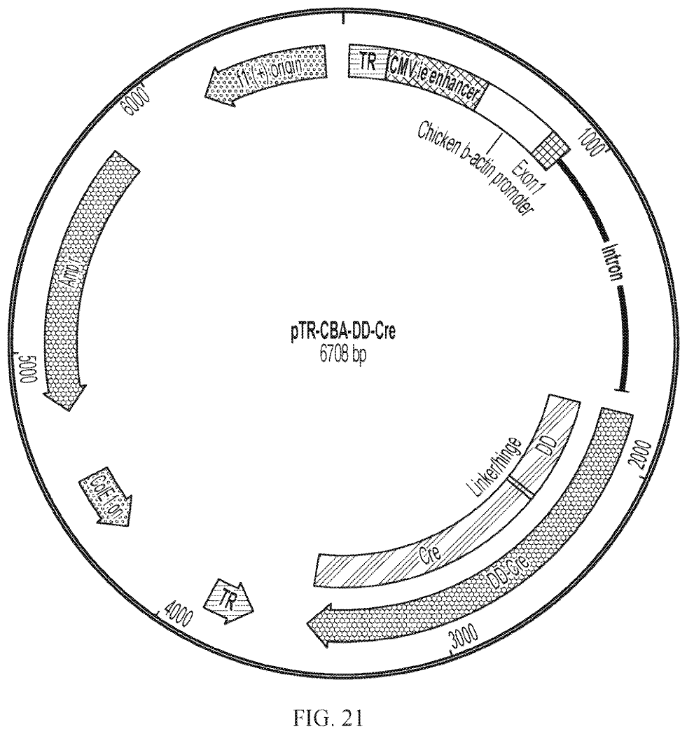

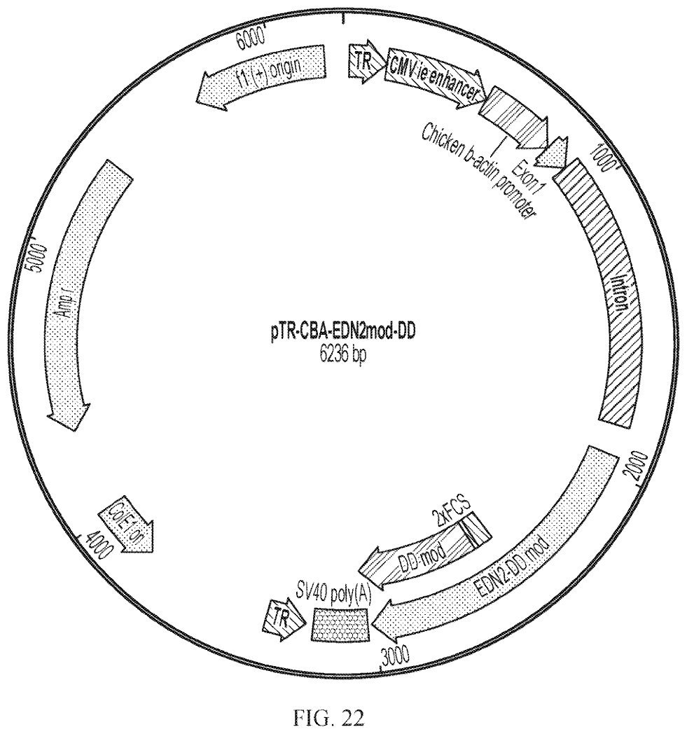

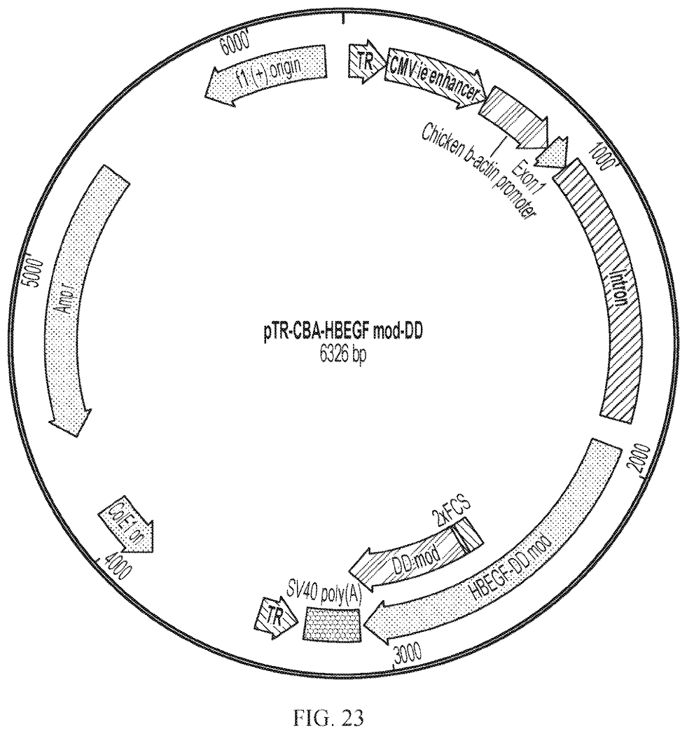

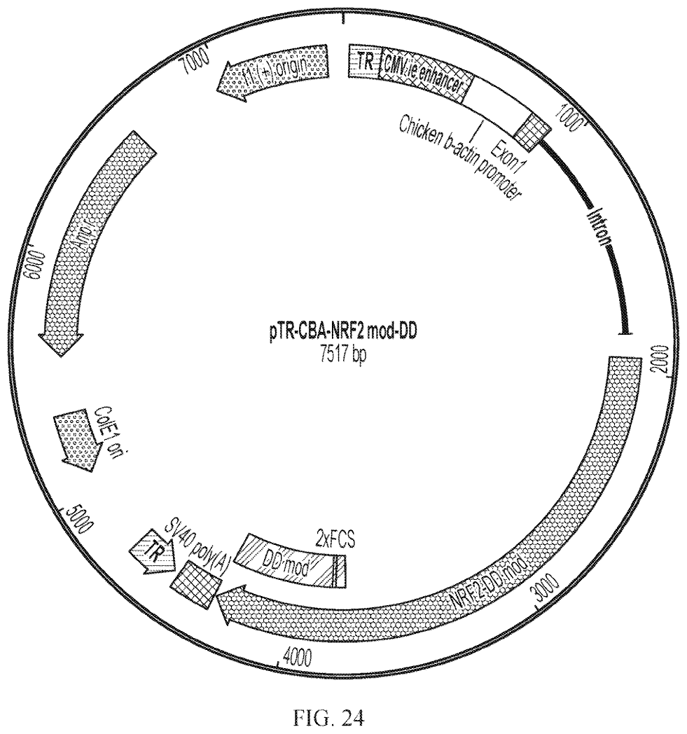

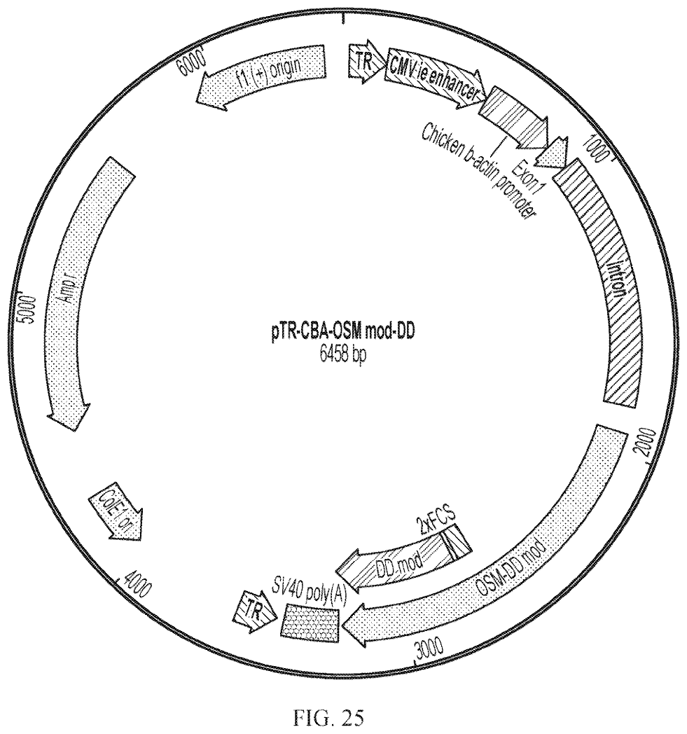

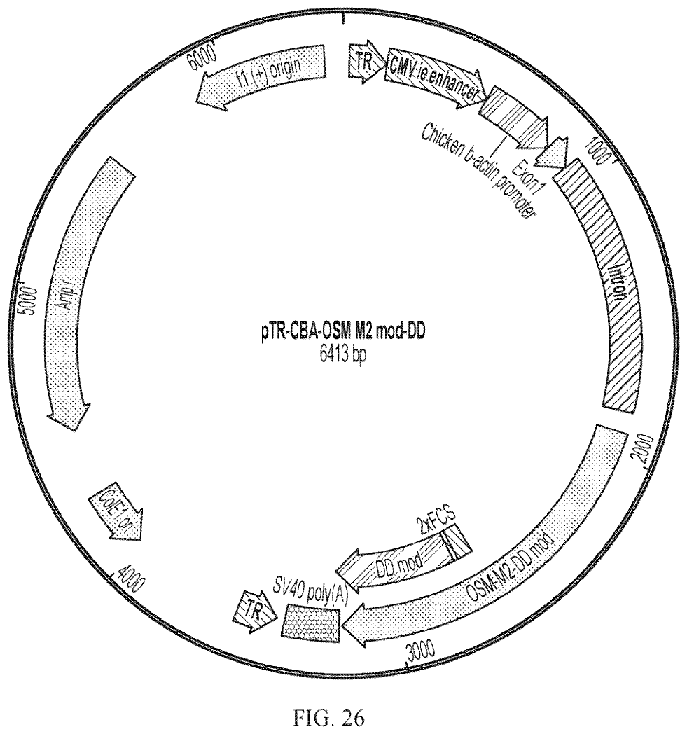

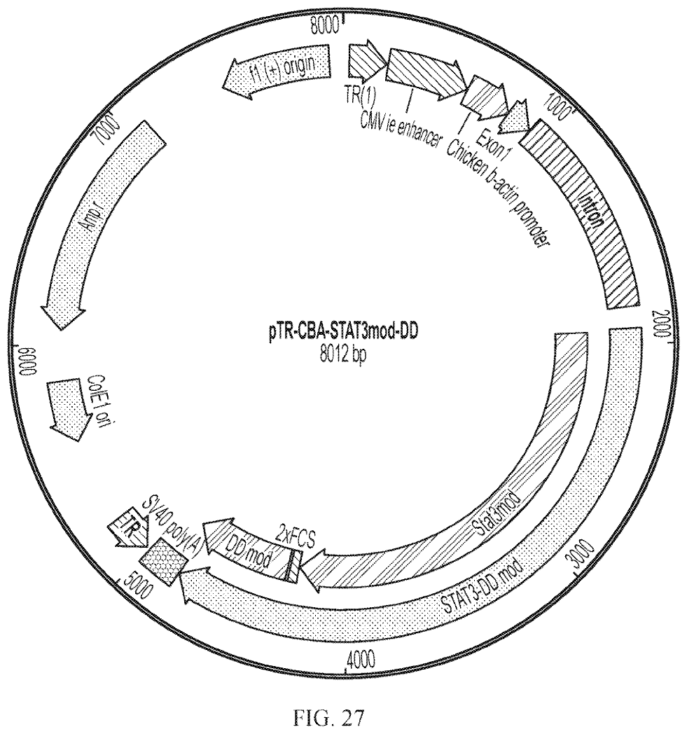

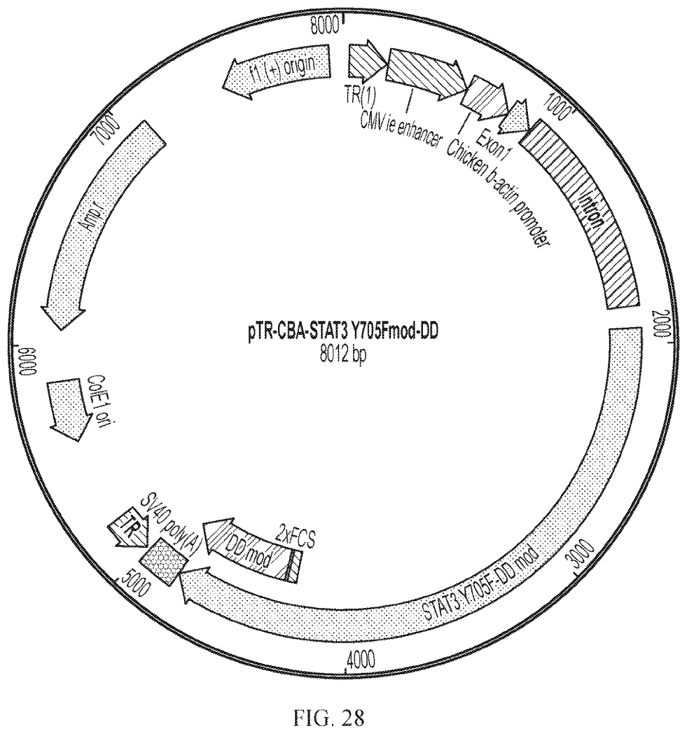

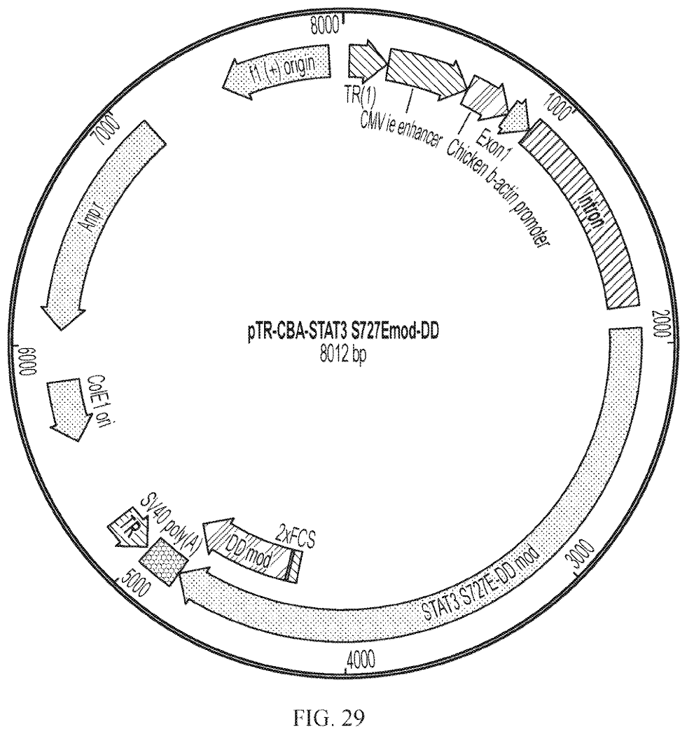

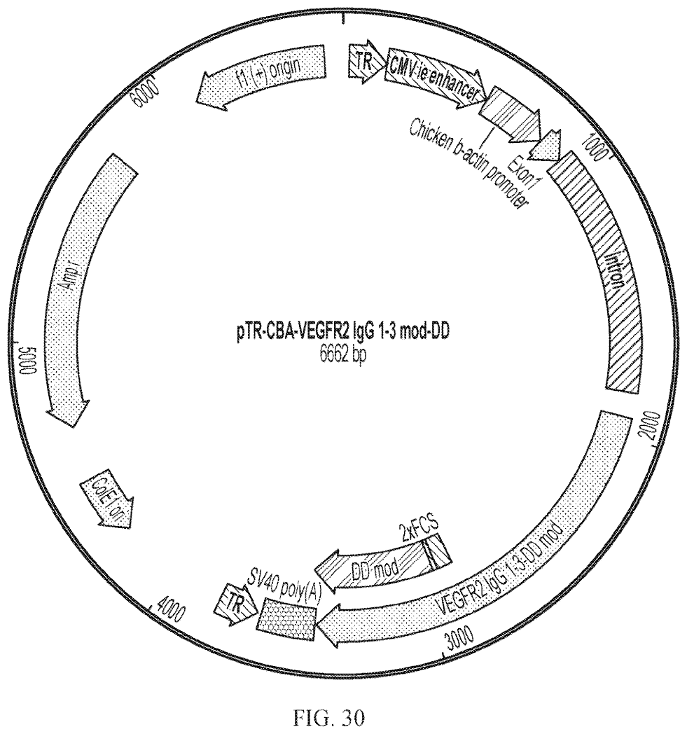

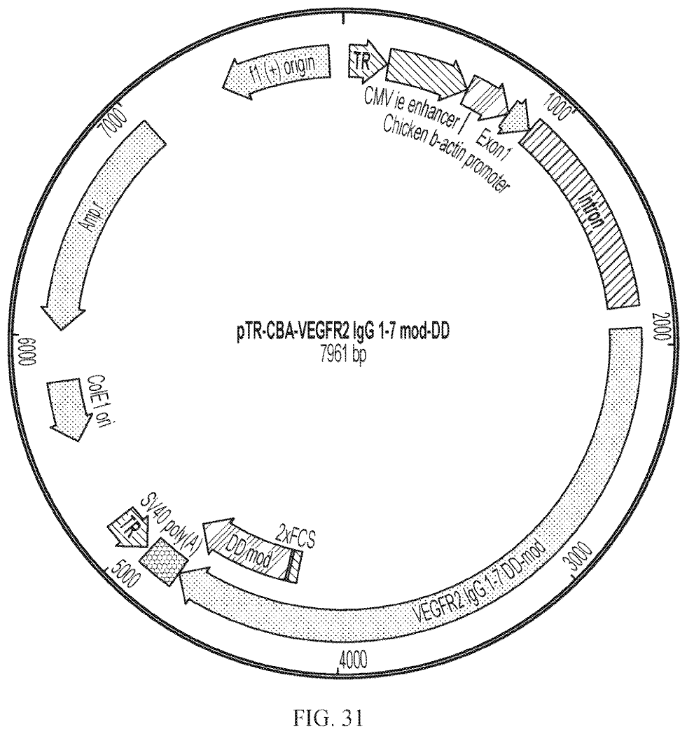

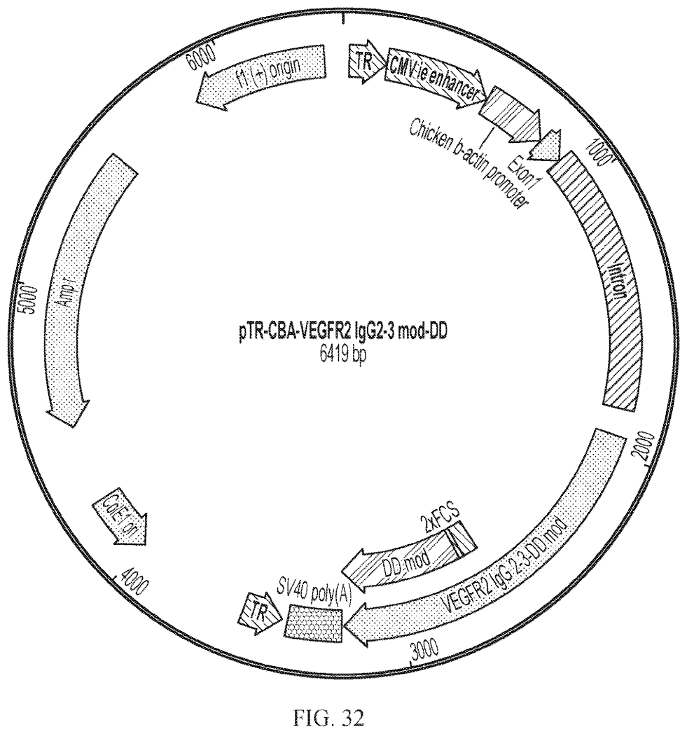

Additional rAAV vectors targeting ASCL1, CNTF, EDN2, HBEGF, NRF2, OSM, OSM-M2, STAT3, STAT3 Y705F, STAT3 S727E, Cre recombinase, VEGFR2 IgG loops 1-3, VEGFR2 IgG loops 1-7, and VEGFR2 IgG loops 2-3 of VEGFR2 were produced. Each rAAV vector comprises a DHFR destabilization domain (DD), which allows for tunable expression based on the presence or absence of TMP in a cell. FIGS. 19-32 show schematics depicting plasmids encoding the rAAV vectors.

SEQUENCES
>Human Leukemia Inhibitory Factor (hLIF) Nucleic Acid Sequence
(SEQ ID NO: 1)
ATGAAGGTCTTGGCGGCAGGAGTTGTGCCCCTGCTGTTGGTTCTGCACTGGAAACATGGGGCGGGGAGCCCCCTCCCCATCACCCCTGTC
AACGCCACCTGTGCCATACGCCACCCATGTCACAACAACCTCATGAACCAGATCAGGAGCCAACTGGCACAGCTCAATGGCAGTGCCAAT
GCCCTCTTTATTCTCTATTACACAGCCCAGGGGGAGCCGTTCCCCAACAACCTGGACAAGCTATGTGGCCCCAACGTGACGGACTTCCCG
CCCTTCCACGCCAACGGCACGGAGAAGGCCAAGCTGGTGGAGCTGTACCGCATAGTCGTGTACCTTGGCACCTCCCTGGGCAACATCACC
CGGGACCAGAAGATCCTCAACCCCAGTGCCCTCAGCCTCCACAGCAAGCTCAACGCCACCGCCGACATCCTGCGAGGCCTCCTTAGCAAC
GTGCTGTGCCGCCTGTGCAGCAAGTACCACGTGGGCCATGTGGACGTGACCTACGGCCCTGACACCTCGGGTAAGGATGTCTTCCAGAAG
AAGAAGCTGGGCTGTCAACTCCTGGGGAAGTATAAGCAGATCATCGCCGTGTTGGCCCAGGCCTTCTAG
>Human Leukemia Inhibitory Factor (hLIF) Amino Acid Sequence
(SEQ ID NO: 2)
MKVLAAGVVPLLLVLHWKHGAGSPLPITPVNATCAIRHPCHNNLMNQIRSQLAQLNGSANALFILYYTAQGEPFPNNLDKLCGPNVTDFP
PFHANGTEKAKLVELYRIVVYLGTSLGNITRDQKILNPSALSLHSKLNATADILRGLLSNVLCRLCSKYHVGHVDVTYGPDTSGKDVFQK
KKLGCQLLGKYKQIIAVLAQAF
>RPF2 Nucleic Acid Sequence; (Bold: Destabilization domain; Italic: 2xFCS linker)
(SEQ ID NO: 3)
atgaaagtgctggctgccggcgtggttcctctgctgctggtacttcactggaagcacggtgccggctcaccactccccatcaccccagta
aacgcgacctgcgctattagacacccatgtcacaacaacctgatgaaccagattcgctcccaactggcccagctgaacgggagtgctaat
gctctgttcatcttgtattacacagcccaaggagaaccctttcccaataaccttgataaactgtgcggccccaatgtgaccgatttcccc
cccttccatgctaacgggacggagaaagctaaactggtcgagctgtaccggatagttgtgtacttgggcacatcactcggcaacattaca
cgggatcagaagatcctgaacccttctgccctttctttgcatagcaagctgaacgccacggctgacatactgaggggtctgctttctaat
gtgttgtgccggctttgctctaagtaccatgtaggccatgtagatgtgacgtacggtccggatacttcagggaaagacgtattccagaaa
aagaagttggggtgccagctgctggggaaatataagcagatcatagccgttctggcccaggcttttaccagatctcgcaagaagcgcagc
accagatctcgcaagaagcgcagcggggcccccatgatatctctgatcgctgctcttgctgtggactacgtgattggtatggagaatgct
atgccctggaatctgcctgcagacctggcatggtttaagaggaacacccttaataaacctgtaatcatgggacgacatacatgggagagt
atcggcaggccattgcccgggaggaagaatataattctgagttcccagccttctactgatgatagggtaacttgggtcaagagcgtcgac
gaggccatcgccgcttgcggggatgtgcctgaaatcatggttatcgggggcggacgggtgattgagcaatttctgcccaaggcacagaag
ctgtatctgacccatatagacgccgaggtggagggagatacacactttcccgactatgagcccgatgattgggagagtgtctttagcgaa
tttcatgacgccgacgcacagaattcccatagctactgcttcgaaattctcgaacggcgctag
>RPF2 Amino Acid Sequence; (Bold: Destabilization domain; Italic: 2xFCS linker)
(SEQ ID NO: 4)
MKVLAAGVVPLLLVLHWKHGAGSPLPITPVNATCAIRHPCHNNLMNQIRSQLAQLNGSANALFILYYTAQGEPFPNNLDKLCGPNVTDFP
PFHANGTEKAKLVELYRIVVYLGTSLGNITRDQKILNPSALSLHSKLNATADILRGLLSNVLCRLCSKYHVGHVDVTYGPDTSGKDVFQK
KKLGCQLLGKYKQIIAVLAQAFTRSRKKRSTRSRKKRSGAPMISLIAALAVDYVIGMENAMPWNLPADLAWFKRNTLNKPVIMGRHTWES
IGRPLPGRKNIILSSQPSTDDRVTWVKSVDEAIAACGDVPEIMVIGGGRVIEQFLPKAQKLYLTHIDAEVEGDTHFPDYEPDDWESVFSE
FHDADAQNSHSYCFEILERR*
> ASCL1-DD amino acid sequence
(SEQ ID NO: 5)
MESSAKMESGGAGQQPQPQPQQPFLPPAACFFATAAAAAAAAAAAAAQSAQQQQQQQQQQQQAPQLRPAADGQPSGGGHKSAPKQVKRQR
SSSPELMRCKRRLNFSGFGYSLPQQQPAAVARRNERERNRVKLVNLGFATLREHVPNGAANKKMSKVETLRSAVEYlRALQQLLDEHDAV
SAAFQAGVLSPTISPNYSNDLNSMAGSPVSSYSSDEGSYDPLSPEEQELLDFTNWFTRSRKKRSTRSRKKRSGAPMISLIAALAVDYVIG
MENAMPWNLPADLAWFKRNTLNKPVIMGRHTWESIGRPLPGRKNIILSSQPSTDDRVTWVKSVDEAIAACGDVPEIMVIGGGRVIEQFLP
KAQKLYLTHIDAEVEGDTHFPDYEPDDWESVFSEFHDADAQNSHSYCFEILERR*
> rAAV vector encoding ASCL1 (TMP-regulated transcription factor ASCL1)
(SEQ ID NO: 6)
ttggccactccctctctgcgcgctcgctcgctcactgaggccgggcgaccaaaggtcgcccgacgcccgggctttgcccgggcggcctca
gtgagcgagcgagcgcgcagagagggagtggccaactccatcactaggggttcctcagatctgaattcggtacctagttattaatagtaa
tcaattacggggtcattagttcatagcccatatatggagttccgcgttacataacttacggtaaatggcccgcctggctgaccgcccaac
gacccccgcccattgacgtcaataatgacgtatgttcccatagtaacgccaatagggactttccattgacgtcaatgggtggagtattta
cggtaaactgcccacttggcagtacatcaagtgtatcatatgccaagtacgccccctattgacgtcaatgacggtaaatggcccgcctgg
cattatgcccagtacatgaccttatgggactttcctacttggcagtacatctacgtattagtcatcgctattaccatggtcgaggtgagc
cccacgttctgcttcactctccccatctcccccccctccccacccccaattttgtatttatttattttttaattattttgtgcagcgatg
ggggcggggggggggggggggcgcgcgccaggcggggcggggcggggcgaggggcggggcggggcgaggcggagaggtgcggcggcagcc
aatcagagcggcgcgctccgaaagtttccttttatggcgaggcggcggcggcggcggccctataaaaagcgaagcgcgcggcgggcggga
gtcgctgcgcgctgccttcgccccgtgccccgctccgccgccgcctcgcgccgcccgccccggctctgactgaccgcgttactcccacag
gtgagcgggcgggacggcccttctcctccgggctgtaattagcgcttggtttaatgacggcttgtttcttttctgtggctgcgtgaaagc
cttgaggggctccgggagggccctttgtgcggggggagcggctcggggggtgcgtgcgtgtgtgtgtgcgtggggagcgccgcgtgcggc
tccgcgctgcccggcggctgtgagcgctgcgggcgcggcgcggggctttgtgcgctccgcagtgtgcgcgaggggagcgcggccgggggc
ggtgccccgcggtgcggggggggctgcgaggggaacaaaggctgcgtgcggggtgtgtgcgtgggggggtgagcagggggtgtgggcgcg
tcggtcgggctgcaaccccccctgcacccccctccccgagttgctgagcacggcccggcttcgggtgcggggctccgtacggggcgtggc
gcggggctcgccgtgccgggcggggggtggcggcaggtgggggtgccgggcggggcggggccgcctcgggccggggagggctcgggggag
gggcgcggcggcccccggagcgccggcggctgtcgaggcgcggcgagccgcagccattgccttttatggtaatcgtgcgagagggcgcag
ggacttcctttgtcccaaatctgtgcggagccgaaatctgggaggcgccgccgcaccccctctagcgggcgcggggcgaagcggtgcggc
gccggcaggaaggaaatgggcggggagggccttcgtgcgtcgccgcgccgccgtccccttctccctctccagcctcggggctgtccgcgg
ggggacggctgccttcgggggggacggggcagggcggggttcggcttctggcgtgtgaccggcggctctagagcctctgctaaccatgtt
catgccttcttctttttcctacagctcctgggcaacgtgctggttattgtgctgtctcatcattttggcaaagaattcctcgaagatcta
ggcaacgcgtctcgaggcggccgccgccaccatggagtcttctgctaaaatggaaagcggaggagcaggtcagcagccgcagccacagcc
ccaacagccgtttctccctccggccgcatgctttttcgcaacagcagccgcagctgcggccgctgccgctgctgctgccgctcaatcagc
ccaacagcagcaacaacaacagcaacagcagcaacaggccccacaactgagaccagcagctgacgggcaaccctcagggggtggacataa
gagtgccccaaagcaagttaagcgccaacgctcaagttccccggagctgatgcggtgtaagagacgcctgaactttagcgggttcggcta
cagcctgccccaacagcaaccagcagccgtagccaggagaaatgaaagggagcgcaatcgggttaagctcgtcaacctcggcttcgcaac
tctgcgcgaacacgtcccaaatggagcggctaacaagaaaatgagtaaagttgagactctccgcagtgctgtcgaatatattcgggcgct
tcaacaacttctcgatgagcacgatgcagtgtctgccgcgtttcaggccggggtcttgtctccgaccatttccccgaactactccaatga
cctgaactctatggcaggttctcccgtctcttcctattcttctgatgaaggctcatacgatcctctgagtcctgaggaacaggaattgtt
ggatttcacgaactggttcaccagatctcgcaagaagcgcagcaccagatctcgcaagaagcgcagcggggcccccatgatatctctgat
cgctgctcttgctgtggactacgtgattggtatggagaatgctatgccctggaatctgcctgcagacctggcatggtttaagaggaacac
ccttaataaacctgtaatcatgggacgacatacatgggagagtatcggcaggccattgcccgggaggaagaatataattctgagttccca
gccttctactgatgatagggtaacttgggtcaagagcgtcgacgaggccatcgccgcttgcggggatgtgcctgaaatcatggttatcgg
gggcggacgggtgattgagcaatttctgcccaaggcacagaagctgtatctgacccatatagacgccgaggtggagggagatacacactt
tcccgactatgagcccgatgattgggagagtgtctttagcgaatttcatgacgccgacgcacagaattcccatagctactgcttcgaaat
tctcgaacggcgctaggcggccgcgcggatccagacatgataagatacattgatgagtttggacaaaccacaactagaatgcagtgaaaa
aaatgctttatttgtgaaatttgtgatgctattgctttatttgtaaccattataagctgcaataaacaagttaacaacaacaattgcatt
cattttatgtttcaggttcagggggaggtgtgggaggttttttagtcgactggggagagatctgaggaacccctagtgatggagttggcc
actccctctctgcgcgctcgctcgctcactgaggccgcccgggcaaagcccgggcgtcgggcgacctttggtcgcccggcctcagtgagc
gagcgagcgcgcagagagggagtggccaac
>CNTF-DD amino acid sequence
(SEQ ID NO: 7)
MAFTEHSPLTPHRRDLCSRSIWLARKIRSDLTALTESYVKHQGLNKNINLDSADGMPVASTDQWSELTEAERLQENLQAYRTFHVLLARL
LEDQQVHFTPTEGDFHQAIHTLLLQVAAFAYQIEELMILLEYKIPRNEADGMPINVGDGGLFEKKLWGLKVLQELSQWTVRSIHDLRFIS
SHQTGIPARGSHYIANNKKMTRSRKKRSTRSRKKRSGAPMISLIAALAVDYVIGMENAMPWNLPADLAWFKRNTLNKPVIMGRHTWESIG
RPLPGRKNIILSSQPSTDDRVTWVKSVDEAIAACGDVPEIMVIGGGRVIEQFLPKAQKLYLTHIDAEVEGDTHFPDYEPDDWESVFSEFH
DADAQNSHSYCFEILERR*
>rAAV vector encoding CNTF (TMP-regulated protective factor CNTF)
(SEQ ID NO: 8)
ttggccactccctctctgcgcgctcgctcgctcactgaggccgggcgaccaaaggtcgcccgacgcccgggctttgcccgggcggcctca
gtgagcgagcgagcgcgcagagagggagtggccaactccatcactaggggttcctcagatctgaattcggtacctagttattaatagtaa
tcaattacggggtcattagttcatagcccatatatggagttccgcgttacataacttacggtaaatggcccgcctggctgaccgcccaac
gacccccgcccattgacgtcaataatgacgtatgttcccatagtaacgccaatagggactttccattgacgtcaatgggtggagtattta
cggtaaactgcccacttggcagtacatcaagtgtatcatatgccaagtacgccccctattgacgtcaatgacggtaaatggcccgcctgg
cattatgcccagtacatgaccttatgggactttcctacttggcagtacatctacgtattagtcatcgctattaccatggtcgaggtgagc
cccacgttctgcttcactctccccatctcccccccctccccacccccaattttgtatttatttattttttaattattttgtgcagcgatg
ggggcggggggggggggggggcgcgcgccaggcggggcggggcggggcgaggggcggggcggggcgaggcggagaggtgcggcggcagcc
aatcagagcggcgcgctccgaaagtttccttttatggcgaggcggcggcggcggcggccctataaaaagcgaagcgcgcggcgggcggga
gtcgctgcgcgctgccttcgccccgtgccccgctccgccgccgcctcgcgccgcccgccccggctctgactgaccgcgttactcccacag
gtgagcgggcgggacggcccttctcctccgggctgtaattagcgcttggtttaatgacggcttgtttcttttctgtggctgcgtgaaagc
cttgaggggctccgggagggccctttgtgcggggggagcggctcggggggtgcgtgcgtgtgtgtgtgcgtggggagcgccgcgtgcggc
tccgcgctgcccggcggctgtgagcgctgcgggcgcggcgcggggctttgtgcgctccgcagtgtgcgcgaggggagcgcggccgggggc
ggtgccccgcggtgcggggggggctgcgaggggaacaaaggctgcgtgcggggtgtgtgcgtgggggggtgagcagggggtgtgggcgcg
tcggtcgggctgcaaccccccctgcacccccctccccgagttgctgagcacggcccggcttcgggtgcggggctccgtacggggcgtggc
gcggggctcgccgtgccgggcggggggtggcggcaggtgggggtgccgggcggggcggggccgcctcgggccggggagggctcgggggag
gggcgcggcggcccccggagcgccggcggctgtcgaggcgcggcgagccgcagccattgccttttatggtaatcgtgcgagagggcgcag
ggacttcctttgtcccaaatctgtgcggagccgaaatctgggaggcgccgccgcaccccctctagcgggcgcggggcgaagcggtgcggc
gccggcaggaaggaaatgggcggggagggccttcgtgcgtcgccgcgccgccgtccccttctccctctccagcctcggggctgtccgcgg
ggggacggctgccttcgggggggacggggcagggcggggttcggcttctggcgtgtgaccggcggctctagagcctctgctaaccatgtt
catgccttcttctttttcctacagctcctgggcaacgtgctggttattgtgctgtctcatcattttggcaaagaattcctcgaagatcta
ggcaacgcgtctcgaggcggccgccgccaccatggcctttactgagcatagccccttgacaccgcacagaagggatctttgctccaggtc
catctggctcgcacggaagatcaggtcagacctgacagccctgaccgaatcatatgtaaagcaccagggcctgaataaaaacattaacct
cgacagcgcagacggaatgccggtggcctcaacagatcagtggtccgaactgacggaggcggagcggttgcaagaaaacttgcaggcgta
tcgaacgtttcacgtcctgcttgcccggcttcttgaagaccagcaggttcatttcacgcctacagagggagatttccatcaagcaatcca
cactttgctcctgcaagtcgcggcctttgcctatcagattgaggaactcatgattcttctcgaatataagattccacggaatgaggcgga
tgggatgccgattaacgtgggcgacggagggttgtttgagaaaaaactctggggtttgaaagttctgcaagaattgagtcagtggactgt
gcgatctatccacgacttgaggttcatctcatcccatcaaacagggatacctgcaagaggctctcattatatagcgaataataaaaaaat
gaccagatctcgcaagaagcgcagcaccagatctcgcaagaagcgcagcggggcccccatgatatctctgatcgctgctcttgctgtgga
ctacgtgattggtatggagaatgctatgccctggaatctgcctgcagacctggcatggtttaagaggaacacccttaataaacctgtaat
catgggacgacatacatgggagagtatcggcaggccattgcccgggaggaagaatataattctgagttcccagccttctactgatgatag
ggtaacttgggtcaagagcgtcgacgaggccatcgccgcttgcggggatgtgcctgaaatcatggttatcgggggcggacgggtgattga
gcaatttctgcccaaggcacagaagctgtatctgacccatatagacgccgaggtggagggagatacacactttcccgactatgagcccga
tgattgggagagtgtctttagcgaatttcatgacgccgacgcacagaattcccatagctactgcttcgaaattctcgaacggcgctaggc
ggccgcgcggatccagacatgataagatacattgatgagtttggacaaaccacaactagaatgcagtgaaaaaaatgctttatttgtgaa
atttgtgatgctattgctttatttgtaaccattataagctgcaataaacaagttaacaacaacaattgcattcattttatgtttcaggtt
cagggggaggtgtgggaggttttttagtcgactggggagagatctgaggaacccctagtgatggagttggccactccctctctgcgcgct
cgctcgctcactgaggccgcccgggcaaagcccgggcgtcgggcgacctttggtcgcccggcctcagtgagcgagcgagcgcgcagagag
ggagtggccaac
>Cre recombinase-DD amino acid sequence
(SEQ ID NO: 9)
MISLIAALAVDYVIGMENAMPWNLPADLAWFKRNTLNKPVIMGRHTWESIGRPLPGRKNIILSSQPSTDDRVTWVKSVDEAIAACGDVPE
IMVIGGGRVIEQFLPKAQKLYLTHIDAEVEGDTHFPDYEPDDWESVFSEFHDADAQNSHSYCFEILERRGAPKKKRKVSNLLTVHQNLPA
LPVDATSDEVRKNLMDMFRDRQAFSEHTWKMLLSVCRSWAAWCKLNNRKWFPAEPEDVRDYLLYLQARGLAVKTIQQHLGQLNMLHRRSG
LPRPSDSNAVSLVMRRIRKENVDAGERAKQALAFERTDFDQVRSLMENSDRCQDIRNLAFLGIAYNTLLRIAEIARIRVKDISRTDGGRM
LIHIGRTKTLVSTAGVEKALSLGVTKLVERWISVSGVADDPNNYLFCRVRKNGVAAPSATSQLSTRALEGIFEATHRLIYGAKDDSGQRY
LAWSGHSARVGAARDMARAGVSIPEIMQAGGWTNVNIVMNYIRNLDSETGAMVRLLEDGD*
>rAAV vector encoding Cre (TMP-Regulated Cre recombinase)
(SEQ ID NO: 10)
ttggccactccctctctgcgcgctcgctcgctcactgaggccgggcgaccaaaggtcgcccgacgcccgggctttgcccgggcggcctca
gtgagcgagcgagcgcgcagagagggagtggccaactccatcactaggggttcctcagatctgaattcggtacctagttattaatagtaa
tcaattacggggtcattagttcatagcccatatatggagttccgcgttacataacttacggtaaatggcccgcctggctgaccgcccaac
gacccccgcccattgacgtcaataatgacgtatgttcccatagtaacgccaatagggactttccattgacgtcaatgggtggagtattta
cggtaaactgcccacttggcagtacatcaagtgtatcatatgccaagtacgccccctattgacgtcaatgacggtaaatggcccgcctgg
cattatgcccagtacatgaccttatgggactttcctacttggcagtacatctacgtattagtcatcgctattaccatggtcgaggtgagc
cccacgttctgcttcactctccccatctcccccccctccccacccccaattttgtatttatttattttttaattattttgtgcagcgatg
ggggcggggggggggggggggcgcgcgccaggcggggcggggcggggcgaggggcggggcggggcgaggcggagaggtgcggcggcagcc
aatcagagcggcgcgctccgaaagtttccttttatggcgaggcggcggcggcggcggccctataaaaagcgaagcgcgcggcgggcggga
gtcgctgcgcgctgccttcgccccgtgccccgctccgccgccgcctcgcgccgcccgccccggctctgactgaccgcgttactcccacag
gtgagcgggcgggacggcccttctcctccgggctgtaattagcgcttggtttaatgacggcttgtttcttttctgtggctgcgtgaaagc
cttgaggggctccgggagggccctttgtgcggggggagcggctcggggggtgcgtgcgtgtgtgtgtgcgtggggagcgccgcgtgcggc
tccgcgctgcccggcggctgtgagcgctgcgggcgcggcgcggggctttgtgcgctccgcagtgtgcgcgaggggagcgcggccgggggc
ggtgccccgcggtgcggggggggctgcgaggggaacaaaggctgcgtgcggggtgtgtgcgtgggggggtgagcagggggtgtgggcgcg
tcggtcgggctgcaaccccccctgcacccccctccccgagttgctgagcacggcccggcttcgggtgcggggctccgtacggggcgtggc
gcggggctcgccgtgccgggcggggggtggcggcaggtgggggtgccgggcggggcggggccgcctcgggccggggagggctcgggggag
gggcgcggcggcccccggagcgccggcggctgtcgaggcgcggcgagccgcagccattgccttttatggtaatcgtgcgagagggcgcag
ggacttcctttgtcccaaatctgtgcggagccgaaatctgggaggcgccgccgcaccccctctagcgggcgcggggcgaagcggtgcggc
gccggcaggaaggaaatgggcggggagggccttcgtgcgtcgccgcgccgccgtccccttctccctctccagcctcggggctgtccgcgg
ggggacggctgccttcgggggggacggggcagggcggggttcggcttctggcgtgtgaccggcggctctagagcctctgctaaccatgtt
catgccttcttctttttcctacagctcctgggcaacgtgctggttattgtgctgtctcatcattttggcaaagaattcctcgaagatcta
ggcctgcaggcggccgccgccaccatgatctctctgattgccgctctggccgtggactacgtgatcgggatggaaaacgctatgccatgg
aatctgcccgccgatctggcttggttcaagaggaacaccctgaacaagccagtgatcatgggcagacacacttgggagtccattggccgg
cccctgcctggacgcaagaacatcattctgagctcccagccctctaccgacgacagggtgacatgggtgaaaagtgtggacgaagccatt
gccgcttgcggagatgtgcccgagatcatggtcatcggcggagggagagtgatcgagcagttcctgcctaaggcccagaaactgtacctg
actcacattgacgctgaggtggaaggggacacccattttcctgattatgagccagacgattgggaaagcgtgttctccgagtttcacgac
gccgatgctcagaattctcatagttattgctttgagatcctggaaaggagaggcgcgcctaagaagaagaggaaggtgtccaatttactg
accgtacaccaaaatttgcctgcattaccggtcgatgcaacgagtgatgaggttcgcaagaacctgatggacatgttcagggatcgccag
gcgttttctgagcatacctggaaaatgcttctgtccgtttgccggtcgtgggcggcatggtgcaagttgaataaccggaaatggtttccc
gcagaacctgaagatgttcgcgattatcttctatatcttcaggcgcgcggtctggcagtaaaaactatccagcaacatttgggccagcta
aacatgcttcatcgtcggtccgggctgccacgaccaagtgacagcaatgctgtttcactggttatgcggcggatccgaaaagaaaacgtt
gatgccggtgaacgtgcaaaacaggctctagcgttcgaacgcactgatttcgaccaggttcgttcactcatggaaaatagcgatcgctgc
caggatatacgtaatctggcatttctggggattgcttataacaccctgttacgtatagccgaaattgccaggatcagggttaaagatatc
tcacgtactgacggtgggagaatgttaatccatattggcagaacgaaaacgctggttagcaccgcaggtgtagagaaggcacttagcctg
ggggtaactaaactggtcgagcgatggatttccgtctctggtgtagctgatgatccgaataactacctgttttgccgggtcagaaaaaat
ggtgttgccgcgccatctgccaccagccagctatcaactcgcgccctggaagggatttttgaagcaactcatcgattgatttacggcgct
aaggatgactctggtcagagatacctggcctggtctggacacagtgcccgtgtcggagccgcgcgagatatggcccgcgctggagtttca
ataccggagatcatgcaagctggtggctggaccaatgtaaatattgtcatgaactatatccgtaacctggatagtgaaacaggggcaatg
gtgcgcctgctggaagatggcgattaggtcgactagagctcgctgatcagcctcgactgtgccttctagttgccagccatctgttgtttg
cccctcccccgtgccttccttgaccctggaaggtgccactcccactgtcctttcctaataaaatgaggaaattgcatcgcattgtctgag
taggtgtcattctattctggggggtggggtggggcaggacagcaagggggaggattgggaagacaatagcaggcatgctggggagagatc
tgaggaacccctagtgatggagttggccactccctctctgcgcgctcgctcgctcactgaggccgcccgggcaaagcccgggcgtcgggc
gacctttggtcgcccggcctcagtgagcgagcgagcgcgcagagagggagtggccaac
>EDN2-DD amino acid sequence
(SEQ ID NO: 11)
MVSVPTTWCSVALALLVALHEGKGQAAATLEQPASSSHAQGTHLRLRRCSCSSWLDKECVYFCHLDIIWVNTPEQTAPYGLGNPPRRRRR
SLPRRCQCSSARDPACATFCLRRPWTEAGAVPSRKSPADVFQTGKTGATTGELLQRLRDISTVKSLFAKRQQEAMREPRSTHSRWRKRTR
SRKKRSTRSRKKRSGAPMISLIAALAVDYVIGMENAMPWNLPADLAWFKRNTLNKPVIMGRHTWESIGRPLPGRKNIILSSQPSTDDRVT
WVKSVDEAIAACGDVPEIMVIGGGRVIEQFLPKAQKLYLTHIDAEVEGDTHFPDYEPDDWESVFSEFHDADAQNSHSYCFEILERR*
>rAAV vector encoding EDN2 (TMP-regulated protective factor EDN2)
(SEQ ID NO: 12)
ttggccactccctctctgcgcgctcgctcgctcactgaggccgggcgaccaaaggtcgcccgacgcccgggctttgcccgggcggcctca
gtgagcgagcgagcgcgcagagagggagtggccaactccatcactaggggttcctcagatctgaattcggtacctagttattaatagtaa
tcaattacggggtcattagttcatagcccatatatggagttccgcgttacataacttacggtaaatggcccgcctggctgaccgcccaac
gacccccgcccattgacgtcaataatgacgtatgttcccatagtaacgccaatagggactttccattgacgtcaatgggtggagtattta
cggtaaactgcccacttggcagtacatcaagtgtatcatatgccaagtacgccccctattgacgtcaatgacggtaaatggcccgcctgg
cattatgcccagtacatgaccttatgggactttcctacttggcagtacatctacgtattagtcatcgctattaccatggtcgaggtgagc
cccacgttctgcttcactctccccatctcccccccctccccacccccaattttgtatttatttattttttaattattttgtgcagcgatg
ggggcggggggggggggggggcgcgcgccaggcggggcggggcggggcgaggggcggggcggggcgaggcggagaggtgcggcggcagcc
aatcagagcggcgcgctccgaaagtttccttttatggcgaggcggcggcggcggcggccctataaaaagcgaagcgcgcggcgggcggga
gtcgctgcgcgctgccttcgccccgtgccccgctccgccgccgcctcgcgccgcccgccccggctctgactgaccgcgttactcccacag
gtgagcgggcgggacggcccttctcctccgggctgtaattagcgcttggtttaatgacggcttgtttcttttctgtggctgcgtgaaagc
cttgaggggctccgggagggccctttgtgcggggggagcggctcggggggtgcgtgcgtgtgtgtgtgcgtggggagcgccgcgtgcggc
tccgcgctgcccggcggctgtgagcgctgcgggcgcggcgcggggctttgtgcgctccgcagtgtgcgcgaggggagcgcggccgggggc
ggtgccccgcggtgcggggggggctgcgaggggaacaaaggctgcgtgcggggtgtgtgcgtgggggggtgagcagggggtgtgggcgcg
tcggtcgggctgcaaccccccctgcacccccctccccgagttgctgagcacggcccggcttcgggtgcggggctccgtacggggcgtggc
gcggggctcgccgtgccgggcggggggtggcggcaggtgggggtgccgggcggggcggggccgcctcgggccggggagggctcgggggag
gggcgcggcggcccccggagcgccggcggctgtcgaggcgcggcgagccgcagccattgccttttatggtaatcgtgcgagagggcgcag
ggacttcctttgtcccaaatctgtgcggagccgaaatctgggaggcgccgccgcaccccctctagcgggcgcggggcgaagcggtgcggc
gccggcaggaaggaaatgggcggggagggccttcgtgcgtcgccgcgccgccgtccccttctccctctccagcctcggggctgtccgcgg
ggggacggctgccttcgggggggacggggcagggcggggttcggcttctggcgtgtgaccggcggctctagagcctctgctaaccatgtt
catgccttcttctttttcctacagctcctgggcaacgtgctggttattgtgctgtctcatcattttggcaaagaattcctcgaagatcta
ggcaacgcgtctcgaggcggccgccgccaccatggtgagcgttcctacgacatggtgctcagtagctcttgcgcttctggtagcgctcca
tgaaggcaaaggccaagcggccgcaaccctcgagcagccggcttcatcctcccacgcccaaggcacgcacctcagactcagaaggtgttc
ctgttcctcatggctggataaagaatgtgtttatttttgccatttggatattatttgggtcaatacgccagaacaaacggccccttacgg
gcttggtaatccgccacgacggagaaggaggagtctccctcgcagatgccaatgctcatctgcacgcgaccctgcatgcgctacgttctg
cctcagacgaccgtggactgaagctggtgcagtgccaagccgcaaatcccctgccgatgtgttccaaacggggaagaccggggctaccac
tggagaacttctgcaacgcttgcgggacatttctacagtcaaatccctgtttgcaaaaaggcaacaggaggcgatgcgcgagccccgatc
tacccatagccggtggcggaaaagaaccagatctcgcaagaagcgcagcaccagatctcgcaagaagcgcagcggggcccccatgatatc
tctgatcgctgctcttgctgtggactacgtgattggtatggagaatgctatgccctggaatctgcctgcagacctggcatggtttaagag
gaacacccttaataaacctgtaatcatgggacgacatacatgggagagtatcggcaggccattgcccgggaggaagaatataattctgag
ttcccagccttctactgatgatagggtaacttgggtcaagagcgtcgacgaggccatcgccgcttgcggggatgtgcctgaaatcatggt
tatcgggggcggacgggtgattgagcaatttctgcccaaggcacagaagctgtatctgacccatatagacgccgaggtggagggagatac
acactttcccgactatgagcccgatgattgggagagtgtctttagcgaatttcatgacgccgacgcacagaattcccatagctactgctt
cgaaattctcgaacggcgctaggcggccgcgcggatccagacatgataagatacattgatgagtttggacaaaccacaactagaatgcag
tgaaaaaaatgctttatttgtgaaatttgtgatgctattgctttatttgtaaccattataagctgcaataaacaagttaacaacaacaat
tgcattcattttatgtttcaggttcagggggaggtgtgggaggttttttagtcgactggggagagatctgaggaacccctagtgatggag
ttggccactccctctctgcgcgctcgctcgctcactgaggccgcccgggcaaagcccgggcgtcgggcgacctttggtcgcccggcctca
gtgagcgagcgagcgcgcagagagggagtggccaac
>HBEGF-DD amino acid sequence
(SEQ ID NO: 13)
MKLLPSVVLKLFLAAVLSALVTGESLERLRRGLAAGTSNPDPPTVSTDQLLPLGGGRDRKVRDLQEADLDLLRVTLSSKPQALATPNKEE
HGKRKKKGKGLGKKRDPCLRKYKDFCIHGECKYVKELRAPSCICHPGYHGERCHGLSLPVENRLYTYDHTTILAVVAVVLSSVCLLVIVG
LLMFRYHRRGGYDVENEEKVKLGMTNSHTRSRKKRSTRSRKKRSGAPMISLIAALAVDYVIGMENAMPWNLPADLAWFKRNTLNKPVIMG
RHTWESIGRPLPGRKNIILSSQPSTDDRVTWVKSVDEAIAACGDVPEIMVIGGGRVIEQFLPKAQKLYLTHIDAEVEGDTHFPDYEPDDW
ESVFSEFHDADAQNSHSYCFEILERR*
>rAAV vector encoding HBEGF(TMP-regulated growth factor HBEGF)
(SEQ ID NO: 14)
ttggccactccctctctgcgcgctcgctcgctcactgaggccgggcgaccaaaggtcgcccgacgcccgggctttgcccgggcggcctca
gtgagcgagcgagcgcgcagagagggagtggccaactccatcactaggggttcctcagatctgaattcggtacctagttattaatagtaa
tcaattacggggtcattagttcatagcccatatatggagttccgcgttacataacttacggtaaatggcccgcctggctgaccgcccaac
gacccccgcccattgacgtcaataatgacgtatgttcccatagtaacgccaatagggactttccattgacgtcaatgggtggagtattta
cggtaaactgcccacttggcagtacatcaagtgtatcatatgccaagtacgccccctattgacgtcaatgacggtaaatggcccgcctgg
cattatgcccagtacatgaccttatgggactttcctacttggcagtacatctacgtattagtcatcgctattaccatggtcgaggtgagc
cccacgttctgcttcactctccccatctcccccccctccccacccccaattttgtatttatttattttttaattattttgtgcagcgatg
ggggcggggggggggggggggcgcgcgccaggcggggcggggcggggcgaggggcggggcggggcgaggcggagaggtgcggcggcagcc
aatcagagcggcgcgctccgaaagtttccttttatggcgaggcggcggcggcggcggccctataaaaagcgaagcgcgcggcgggcggga
gtcgctgcgcgctgccttcgccccgtgccccgctccgccgccgcctcgcgccgcccgccccggctctgactgaccgcgttactcccacag
gtgagcgggcgggacggcccttctcctccgggctgtaattagcgcttggtttaatgacggcttgtttcttttctgtggctgcgtgaaagc
cttgaggggctccgggagggccctttgtgcggggggagcggctcggggggtgcgtgcgtgtgtgtgtgcgtggggagcgccgcgtgcggc
tccgcgctgcccggcggctgtgagcgctgcgggcgcggcgcggggctttgtgcgctccgcagtgtgcgcgaggggagcgcggccgggggc
ggtgccccgcggtgcggggggggctgcgaggggaacaaaggctgcgtgcggggtgtgtgcgtgggggggtgagcagggggtgtgggcgcg
tcggtcgggctgcaaccccccctgcacccccctccccgagttgctgagcacggcccggcttcgggtgcggggctccgtacggggcgtggc
gcggggctcgccgtgccgggcggggggtggcggcaggtgggggtgccgggcggggcggggccgcctcgggccggggagggctcgggggag
gggcgcggcggcccccggagcgccggcggctgtcgaggcgcggcgagccgcagccattgccttttatggtaatcgtgcgagagggcgcag
ggacttcctttgtcccaaatctgtgcggagccgaaatctgggaggcgccgccgcaccccctctagcgggcgcggggcgaagcggtgcggc
gccggcaggaaggaaatgggcggggagggccttcgtgcgtcgccgcgccgccgtccccttctccctctccagcctcggggctgtccgcgg
ggggacggctgccttcgggggggacggggcagggcggggttcggcttctggcgtgtgaccggcggctctagagcctctgctaaccatgtt
catgccttcttctttttcctacagctcctgggcaacgtgctggttattgtgctgtctcatcattttggcaaagaattcctcgaagatcta
ggcaacgcgtctcgaggcggccgccgccaccatgaaactcctgccgtctgtagtcctgaaactgtttctggctgctgtgttgagtgctct
cgttacgggagagtccttggagaggcttcgacgcggtttggcagcaggtacgtcaaacccagatccccctaccgtgagtacggatcagct
tctgccgctcggcggaggaagggaccgcaaggtgcgcgatctgcaggaggcggaccttgacctgcttcgagttacattgtcctctaagcc
tcaagctctggcgacgccgaacaaagaggagcatggtaagcgaaagaagaaaggcaaagggctgggaaaaaaacgcgacccctgtcttcg
caagtataaggacttctgtattcatggagagtgtaagtatgttaaagagcttcgagctcccagttgcatttgccaccctgggtatcacgg
ggaacgctgtcatggcttgtcattgccagttgaaaatcgcttgtatacgtatgaccataccactatcctcgcagtagtagctgttgtcct
ttccagcgtttgtttgctggtcatcgtcggcttgctgatgtttcggtaccaccgacggggaggttacgatgtagagaacgaggagaaagt
caagctgggcatgacaaatagccataccagatctcgcaagaagcgcagcaccagatctcgcaagaagcgcagcggggcccccatgatatc
tctgatcgctgctcttgctgtggactacgtgattggtatggagaatgctatgccctggaatctgcctgcagacctggcatggtttaagag
gaacacccttaataaacctgtaatcatgggacgacatacatgggagagtatcggcaggccattgcccgggaggaagaatataattctgag
ttcccagccttctactgatgatagggtaacttgggtcaagagcgtcgacgaggccatcgccgcttgcggggatgtgcctgaaatcatggt
tatcgggggcggacgggtgattgagcaatttctgcccaaggcacagaagctgtatctgacccatatagacgccgaggtggagggagatac
acactttcccgactatgagcccgatgattgggagagtgtctttagcgaatttcatgacgccgacgcacagaattcccatagctactgctt
cgaaattctcgaacggcgctaggcggccgcgcggatccagacatgataagatacattgatgagtttggacaaaccacaactagaatgcag
tgaaaaaaatgctttatttgtgaaatttgtgatgctattgctttatttgtaaccattataagctgcaataaacaagttaacaacaacaat
tgcattcattttatgtttc aggttcagggggaggtgtgggaggttattagtcgactggggagagatctgaggaacccctagtgatggag
ttggccactccctctctgcgcgctcgctcgctcactgaggccgcccgggcaaagcccgggcgtcgggcgacctttggtcgcccggcctca
gtgagcgagcgagcgcgcagagagggagtggccaac
>NRF2-DD amino acid sequence
(SEQ ID NO: 15)
MMDLELPPPGLPSQQDMDLIDILWRQDIDLGVSREVFDFSQRRKEYELEKQKKLEKERQEQLQKEQEKAFFAQLQLDEETGEFLPIQPAQ
HIQSETSGSANYSQVAHIPKSDALYFDDCMQLLAQTFPFVDDNEVSSATFQSLVPDIPGHIESPVFIATNQAQSPETSVAQVAPVDLDGM
QQDIEQVWEELLSIPELQCLNIENDKLVETTMVPSPEAKLTEVDNYHFYSSIPSMEKEVGNCSPHFLNAFEDSFSSILSTEDPNQLTVNS
LNSDATVNTDFGDEFYSAFIAEPSISNSMPSPATLSHSLSELLNGPIDVSDLSLCKAFNQNHPESTAEFNDSDSGISLNTSPSVASPEHS
VESSSYGDTLLGLSDSEVEELDSAPGSVKQNGPKTPVHSSGDMVQPLSPSQGQSTHVHDAQCENTPEKELPVSPGHRKTPFTKDKHSSRL
EAHLTRDELRAKALHIPFPVEKIINLPVVDFNEMMSKEQFNEAQLALIRDIRRRGKNKVAAQNCRKRKLENIVELEQDLDHLKDEKEKLL
KEKGENDKSLHLLKKQLSTLYLEVFSMLRDEDGKPYSPSEYSLQQTRDGNVFLVPKSKKPDVKKNTRSRKKRSTRSRKKRSGAPMISLIA
ALAVDYVIGMENAMPWNLPADLAWFKRNTLNKPVIMGRHTWESIGRPLPGRKNIILSSQPSTDDRVTWVKSVDEAIAACGDVPEIMVIGG
GRVIEQFLPKAQKLYLTHIDAEVEGDTHFPDYEPDDWESVFSEFHDADAQNSHSYCFEILERR*
>rAAV vector encoding NRF2 (TMP-regulated transcription factor NRF2)
(SEQ ID NO: 16)
ttggccactccctctctgcgcgctcgctcgctcactgaggccgggcgaccaaaggtcgcccgacgcccgggctttgcccgggcggcctca
gtgagcgagcgagcgcgcagagagggagtggccaactccatcactaggggttcctcagatctgaattcggtacctagttattaatagtaa
tcaattacggggtcattagttcatagcccatatatggagttccgcgttacataacttacggtaaatggcccgcctggctgaccgcccaac
gacccccgcccattgacgtcaataatgacgtatgttcccatagtaacgccaatagggactttccattgacgtcaatgggtggagtattta
cggtaaactgcccacttggcagtacatcaagtgtatcatatgccaagtacgccccctattgacgtcaatgacggtaaatggcccgcctgg
cattatgcccagtacatgaccttatgggactttcctacttggcagtacatctacgtattagtcatcgctattaccatggtcgaggtgagc
cccacgttctgcttcactctccccatctcccccccctccccacccccaattttgtatttatttattttttaattattttgtgcagcgatg
ggggcggggggggggggggggcgcgcgccaggcggggcggggcggggcgaggggcggggcggggcgaggcggagaggtgcggcggcagcc
aatcagagcggcgcgctccgaaagtttccttttatggcgaggcggcggcggcggcggccctataaaaagcgaagcgcgcggcgggcggga
gtcgctgcgcgctgccttcgccccgtgccccgctccgccgccgcctcgcgccgcccgccccggctctgactgaccgcgttactcccacag
gtgagcgggcgggacggcccttctcctccgggctgtaattagcgcttggtttaatgacggcttgtttcttttctgtggctgcgtgaaagc
cttgaggggctccgggagggccctttgtgcggggggagcggctcggggggtgcgtgcgtgtgtgtgtgcgtggggagcgccgcgtgcggc
tccgcgctgcccggcggctgtgagcgctgcgggcgcggcgcggggctttgtgcgctccgcagtgtgcgcgaggggagcgcggccgggggc
ggtgccccgcggtgcggggggggctgcgaggggaacaaaggctgcgtgcggggtgtgtgcgtgggggggtgagcagggggtgtgggcgcg
tcggtcgggctgcaaccccccctgcacccccctccccgagttgctgagcacggcccggcttcgggtgcggggctccgtacggggcgtggc
gcggggctcgccgtgccgggcggggggtggcggcaggtgggggtgccgggcggggcggggccgcctcgggccggggagggctcgggggag
gggcgcggcggcccccggagcgccggcggctgtcgaggcgcggcgagccgcagccattgccttttatggtaatcgtgcgagagggcgcag
ggacttcctttgtcccaaatctgtgcggagccgaaatctgggaggcgccgccgcaccccctctagcgggcgcggggcgaagcggtgcggc
gccggcaggaaggaaatgggcggggagggccttcgtgcgtcgccgcgccgccgtccccttctccctctccagcctcggggctgtccgcgg
ggggacggctgccttcgggggggacggggcagggcggggttcggcttctggcgtgtgaccggcggctctagagcctctgctaaccatgtt
catgccttcttctttttcctacagctcctgggcaacgtgctggttattgtgctgtctcatcattttggcaaagaattcctcgaagatcta
ggcaacgcgtctcgaggcggccgccgccaccatgatggatctcgaacttccccctccaggcctgcctagccagcaagacatggaccttat
cgacatcctgtggcgccaggatattgatctcggtgtgtctagggaagtctttgacttttcccaaaggaggaaggaatatgagcttgagaa
acaaaagaagcttgaaaaggaacggcaagagcagcttcaaaaggagcaggaaaaagcattctttgcgcaattgcagcttgatgaagaaac
cggcgaattccttccaatacaaccagctcaacatatccaatccgagacctcaggttccgcaaattactcccaagtggcacatataccgaa
atccgacgcgttgtacttcgacgattgtatgcagctgctcgcacaaacgttccctttcgtggacgataacgaagtgagttctgctacttt
ccagtcacttgttccagacatccccggacatattgaatccccagtgtttatagcgacaaaccaagcccaatcacctgagacgagtgtcgc
ccaagtagccccagtcgatctggatggtatgcaacaggacattgaacaagtctgggaagagctgcttagcattcctgaactgcagtgcct
gaacatcgaaaacgacaaattggtagagacaactatggtaccctcacctgaggccaagttgaccgaggtggacaattatcatttttactc
ttcaatacctagtatggagaaagaagtcggtaattgttcccctcatttcctgaacgcgttcgaagacagctttagcagcatcttgtccac
agaagacccaaatcaattgacggtaaactccttgaactcagacgcgacagtgaataccgattttggtgatgaattttattcagcatttat
agccgagccaagcatcagtaattctatgcccagccccgcaactctctcccacagtctttcagaattgctcaacggacccatcgatgtgag
tgatctgtccctctgcaaagcgtttaaccaaaaccatccggagagtacggccgagttcaacgacagtgatagtggtatttcactcaatac
ctcaccctccgtggccagtccggagcacagtgttgaatcaagctcctacggggacacattgctgggcctctcagacagcgaagtcgaaga
acttgacagcgcccccggatccgtaaagcaaaatgggccgaaaactccggtgcattcatcaggtgatatggtacagccactttcaccaag
tcagggacaaagtacgcacgtccatgacgcgcaatgtgaaaatactcctgagaaagagctcccggtatcaccggggcaccggaagacccc
ttttactaaggacaaacatagtagtcgcttggaggctcatttgactcgagatgagctccgcgcaaaagcactccatattccatttcccgt
tgaaaagattattaacctcccggtagtggacttcaacgagatgatgtctaaggagcagtttaatgaggcgcagcttgcactgataaggga
catacgacgcagaggtaagaataaggtggctgcccaaaactgcaggaagcgaaagctcgagaacattgtagaacttgagcaggaccttga
tcacctgaaagatgagaaggaaaaattgcttaaagagaaaggtgagaatgataaaagcttgcatcttctcaaaaaacagctgagcacatt
gtatcttgaggtcttcagcatgctcagagatgaagatgggaaaccgtattctccgagcgaatacagtctgcagcagacccgggatggcaa
tgtgttcctcgtacccaaaagcaaaaaacctgatgttaagaagaataccagatctcgcaagaagcgcagcaccagatctcgcaagaagcg
cagcggggcccccatgatatctctgatcgctgctcttgctgtggactacgtgattggtatggagaatgctatgccctggaatctgcctgc
agacctggcatggtttaagaggaacacccttaataaacctgtaatcatgggacgacatacatgggagagtatcggcaggccattgcccgg
gaggaagaatataattctgagttcccagccttctactgatgatagggtaacttgggtcaagagcgtcgacgaggccatcgccgcttgcgg
ggatgtgcctgaaatcatggttatcgggggcggacgggtgattgagcaatttctgcccaaggcacagaagctgtatctgacccatataga
cgccgaggtggagggagatacacactttcccgactatgagcccgatgattgggagagtgtctttagcgaatttcatgacgccgacgcaca
gaattcccatagctactgcttcgaaattctcgaacggcgctaggcggccgcgcggatccagacatgataagatacattgatgagtttgga
caaaccacaactagaatgcagtgaaaaaaatgctttatttgtgaaatttgtgatgctattgctttatttgtaaccattataagctgcaat
aaacaagttaacaacaacaattgcattcattttatgtttcaggttcagggggaggtgtgggaggttttttagtcgactggggagagatct
gaggaacccctagtgatggagttggccactccctctctgcgcgctcgctcgctcactgaggccgcccgggcaaagcccgggcgtcgggcg
acctttggtcgcccggcctcagtgagcgagcgagcgcgcagagagggagtggccaac
>OSM-DD amino acid sequence
(SEQ ID NO: 17)
MGVLLTQRTLLSLVLALLFPSMASMAAIGSCSKEYRVLLGQLQKQTDLMQDTSRLLDPYIRIQGLDVPKLREHCRERPGAFPSEETLRGL
GRRGFLQTLNATLGCVLHRLADLEQRLPKAQDLERSGLNIEDLEKLQMARPNILGLRNNIYCMAQLLDNSDTAEPTKAGRGASQPPTPTP
ASDAFQRKLEGCRFLHGYHRFMHSVGRVFSKWGESPNRSRRHSPHQALRKGVRRTRPSRKGKRLMTRGQLPRTRSRKKRSTRSRKKRSGA
PMISLIAALAVDYVIGMENAMPWNLPADLAWFKRNTLNKPVIMGRHTWESIGRPLPGRKNIILSSQPSTDDRVTWVKSVDEAIAACGDVP
EIMVIGGGRVIEQFLPKAQKLYLTHIDAEVEGDTHFPDYEPDDWESVFSEFHDADAQNSHSYCFEILERR*
>rAAV vector encoding OSM (TMP-regulated protective factor OSM)
(SEQ ID NO: 18)
ttggccactccctctctgcgcgctcgctcgctcactgaggccgggcgaccaaaggtcgcccgacgcccgggctttgcccgggcggcctca
gtgagcgagcgagcgcgcagagagggagtggccaactccatcactaggggttcctcagatctgaattcggtacctagttattaatagtaa
tcaattacggggtcattagttcatagcccatatatggagttccgcgttacataacttacggtaaatggcccgcctggctgaccgcccaac
gacccccgcccattgacgtcaataatgacgtatgttcccatagtaacgccaatagggactttccattgacgtcaatgggtggagtattta
cggtaaactgcccacttggcagtacatcaagtgtatcatatgccaagtacgccccctattgacgtcaatgacggtaaatggcccgcctgg
cattatgcccagtacatgaccttatgggactttcctacttggcagtacatctacgtattagtcatcgctattaccatggtcgaggtgagc
cccacgttctgcttcactctccccatctcccccccctccccacccccaattttgtatttatttattttttaattattttgtgcagcgatg
ggggcggggggggggggggggcgcgcgccaggcggggcggggcggggcgaggggcggggcggggcgaggcggagaggtgcggcggcagcc
aatcagagcggcgcgctccgaaagtttccttttatggcgaggcggcggcggcggcggccctataaaaagcgaagcgcgcggcgggcggga
gtcgctgcgcgctgccttcgccccgtgccccgctccgccgccgcctcgcgccgcccgccccggctctgactgaccgcgttactcccacag
gtgagcgggcgggacggcccttctcctccgggctgtaattagcgcttggtttaatgacggcttgtttcttttctgtggctgcgtgaaagc
cttgaggggctccgggagggccctttgtgcggggggagcggctcggggggtgcgtgcgtgtgtgtgtgcgtggggagcgccgcgtgcggc
tccgcgctgcccggcggctgtgagcgctgcgggcgcggcgcggggctttgtgcgctccgcagtgtgcgcgaggggagcgcggccgggggc
ggtgccccgcggtgcggggggggctgcgaggggaacaaaggctgcgtgcggggtgtgtgcgtgggggggtgagcagggggtgtgggcgcg
tcggtcgggctgcaaccccccctgcacccccctccccgagttgctgagcacggcccggcttcgggtgcggggctccgtacggggcgtggc
gcggggctcgccgtgccgggcggggggtggcggcaggtgggggtgccgggcggggcggggccgcctcgggccggggagggctcgggggag
gggcgcggcggcccccggagcgccggcggctgtcgaggcgcggcgagccgcagccattgccttttatggtaatcgtgcgagagggcgcag
ggacttcctttgtcccaaatctgtgcggagccgaaatctgggaggcgccgccgcaccccctctagcgggcgcggggcgaagcggtgcggc
gccggcaggaaggaaatgggcggggagggccttcgtgcgtcgccgcgccgccgtccccttctccctctccagcctcggggctgtccgcgg
ggggacggctgccttcgggggggacggggcagggcggggttcggcttctggcgtgtgaccggcggctctagagcctctgctaaccatgtt
catgccttcttctttttcctacagctcctgggcaacgtgctggttattgtgctgtctcatcattttggcaaagaattcctcgaagatcta
ggcaacgcgtctcgaggcggccgccgccaccatgggggtgcttttgacacagcgcacactcctgagcttggttctggctctcctgttccc
gtcaatggcttctatggcggccataggctcatgttctaaagaataccgagtgcttctgggccaacttcagaagcagacggacctcatgca
ggatacaagccgccttttggacccatatataaggatccaagggttggatgtgccgaagctgagggaacactgtcgagaacgacctggagc
ttttccaagcgaagaaaccctcagaggtcttgggaggaggggctttttgcagacgctcaacgcgaccctggggtgtgtgctccaccgact
tgcagatctcgagcagcgcctgccaaaagcacaggatctggagcggagtggacttaacatcgaagacctggaaaaattgcagatggctcg
accgaacattcttggccttcgaaacaacatctactgtatggcacagctgctggataactcagatacagccgaacctacaaaagcagggcg
gggcgcgtcccaaccacctactcccacaccagcctccgatgcctttcagagaaagcttgaagggtgtaggtttctccacggctatcatcg
cttcatgcattcagtagggagggtgttctcaaaatggggagagtcacctaatcgatcaaggaggcactcaccccatcaggctcttcggaa
gggcgttcggcgcactcgcccgagcaggaagggtaaaaggctgatgactcggggtcaactgccaaggaccagatctcgcaagaagcgcag
caccagatctcgcaagaagcgcagcggggcccccatgatatctctgatcgctgctcttgctgtggactacgtgattggtatggagaatgc
tatgccctggaatctgcctgcagacctggcatggtttaagaggaacacccttaataaacctgtaatcatgggacgacatacatgggagag
tatcggcaggccattgcccgggaggaagaatataattctgagttcccagccttctactgatgatagggtaacttgggtcaagagcgtcga
cgaggccatcgccgcttgcggggatgtgcctgaaatcatggttatcgggggcggacgggtgattgagcaatttctgcccaaggcacagaa
gctgtatctgacccatatagacgccgaggtggagggagatacacactttcccgactatgagcccgatgattgggagagtgtctttagcga
atttcatgacgccgacgcacagaattcccatagctactgcttcgaaattctcgaacggcgctaggcggccgcgcggatccagacatgata
agatacattgatgagtttggacaaaccacaactagaatgcagtgaaaaaaatgctttatttgtgaaatttgtgatgctattgctttattt
gtaaccattataagctgcaataaacaagttaacaacaacaattgcattcattttatgtttcaggttcagggggaggtgtgggaggttttt
tagtcgactggggagagatctgaggaacccctagtgatggagttggccactccctctctgcgcgctcgctcgctcactgaggccgcccgg
gcaaagcccgggcgtcgggcgacctttggtcgcccggcctcagtgagcgagcgagcgcgcagagagggagtggccaac
>OSM M2-DD variant amino acid sequence
(SEQ ID NO: 19)
MGVLLTQRTLLSLVLALLFPSMASMAAIGSCSKEYRVLLGQLQKQTDLMQDTSRLLDPYIRIQGLDVPKLREHCRERPGAFPSEETLRGL
GRRGFLQTLNATLGCVLHRLADLEQRLGGGNIEDLEKLQMARPNILGLRNNIYCMAQLLDNSDTAEPTKAGRGASQPPTPTPASDAFQRK
LEGCRFLHGYHRFMHSVGRVFSKWGESPNRSRRHSPHQALRKGVRRTRPSRKGKRLMTRSRKKRSTRSRKKRSGAPMISLIAALAVDYVI
GMENAMPWNLPADLAWFKRNTLNKPVIMGRHTWESIGRPLPGRKNIILSSQPSTDDRVTWVKSVDEAIAACGDVPEIMVIGGGRVIEQFL
PKAQKLYLTHIDAEVEGDTHFPDYEPDDWESVFSEFHDADAQNSHSYCFEILERR*
>rAAV vector encoding OSM-M2 (TMP regulated protective factor OSM-M2)
(SEQ ID NO: 20)
ttggccactccctctctgcgcgctcgctcgctcactgaggccgggcgaccaaaggtcgcccgacgcccgggctagcccgggcggcctcag
tgagcgagcgagcgcgcagagagggagtggccaactccatcactaggggacctcagatctgaattcggtacctagttattaatagtaatc
aattacggggtcattagacatagcccatatatggagaccgcgttacataacttacggtaaatggcccgcctggctgaccgcccaacgacc
cccgcccattgacgtcaataatgacgtatgacccatagtaacgccaatagggactaccattgacgtcaatgggtggagtatttacggtaa
actgcccacaggcagtacatcaagtgtatcatatgccaagtacgccccctattgacgtcaatgacggtaaatggcccgcctggcattatg
cccagtacatgaccttatgggactacctacaggcagtacatctacgtattagtcatcgctattaccatggtcgaggtgagccccacgact
gcacactctccccatctcccccccctccccacccccaattagtatttatttattattaattattagtgcagcgatgggggcggggggggg
gggggggcgcgcgccaggcggggcggggcggggcgaggggcggggcggggcgaggcggagaggtgcggcggcagccaatcagagcggcgc
gctccgaaagtaccattatggcgaggcggcggcggcggcggccctataaaaagcgaagcgcgcggcgggcgggagtcgctgcgcgctgcc
acgccccgtgccccgctccgccgccgcctcgcgccgcccgccccggctctgactgaccgcgttactcccacaggtgagcgggcgggacgg
cccactcctccgggctgtaattagcgcaggataatgacggcttgatcattctgtggctgcgtgaaagccagaggggctccgggagggccc
tagtgcggggggagcggctcggggggtgcgtgcgtgtgtgtgtgcgtggggagcgccgcgtgcggctccgcgctgcccggcggctgtgag
cgctgcgggcgcggcgcggggctagtgcgctccgcagtgtgcgcgaggggagcgcggccgggggcggtgccccgcggtgcggggggggct
gcgaggggaacaaaggctgcgtgcggggtgtgtgcgtgggggggtgagcagggggtgtgggcgcgtcggtcgggctgcaaccccccctgc
acccccctccccgagagctgagcacggcccggcttcgggtgcggggctccgtacggggcgtggcgcggggctcgccgtgccgggcggggg
gtggcggcaggtgggggtgccgggcggggcggggccgcctcgggccggggagggctcgggggaggggcgcggcggcccccggagcgccgg
cggctgtcgaggcgcggcgagccgcagccattgccattatggtaatcgtgc1gagagggcgcagggacacctagtcccaaatctgtgcgg
agccgaaatctgggaggcgccgccgcaccccctctagcgggcgcggggcgaagcggtgcggcgccggcaggaaggaaatgggcggggagg
gccacgtgcgtcgccgcgccgccgtccccactccctctccagcctcggggctgtccgcggggggacggctgccacgggggggacggggca
gggcggggacggcactggcgtgtgaccggcggctctagagcctctgctaaccatgacatgccacttcatacctacagctcctgggcaacg
tgctggttattgtgctgtctcatcattaggcaaagaattcctcgaagatctaggcaacgcgtctcgaggcggccgccgccaccatgggtg
tgagctcactcaaaggaccagagtccctggtcctggcactcagacccatctatggcgagtatggcggctattggctcatgctctaaagag
tacagagactgctcggtcaactgcaaaagcaaacggacctgatgcaagatacgtccaggagctcgacccttatattaggatccagggact
cgacgaccgaagcttagagagcactgtcgcgagcgaccaggtgccatcccagtgaagaaacactgaggggtagggcaggcgcggatcagc
agacccttaatgctacattggggtgcgtattgcatcgcctggcagatctcgagcaaagactcggaggtggcaatatcgaggatcagaaaa
gctccagatggctcgcccgaacatactcgggctgaggaataatatatattgtatggctcaactcctcgacaactcagacactgcggagcc
tacgaaagcaggaaggggcgcatcacaaccaccta1cacctactcctgccagtgatgccatcagcgcaagcttgagggctgccgatactc
cacggctatcatcggacatgcactccgaggccgagtgattcaaaatggggggaatctcctaaccgatctagaaggcactcacctcaccag
gcactgcggaaaggcgttaggagaacccggccaagccgcaaaggaaagcgcctgatgaccagatctcgcaagaagcgcagcaccagatct
cgcaagaagcgcagcggggcccccatgatatctctgatcgctgctcttgctgtggactacgtgattggtatggagaatgctatgccctgg
aatctgcctgcagacctggcatggtttaagaggaacacccttaataaacctgtaatcatgggacgacatacatgggagagtatcggcagg
ccattgcccgggaggaagaatataattctgagttcccagccttctactgatgatagggtaacttgggtcaagagcgtcgacgaggccatc
gccgcttgcggggatgtgcctgaaatcatggttatcgggggcggacgggtgattgagcaatttctgcccaaggcacagaagctgtatctg
acccatatagacgccgaggtggagggagatacacactttcccgactatgagcccgatgattgggagagtgtctttagcgaatttcatgac
gccgacgcacagaattcccatagctactgcttcgaaattctcgaacggcgctaggcggccgcgcggatccagacatgataagatacattg
atgagtttggacaaaccacaactagaatgcagtgaaaaaaatgctttatttgtgaaatttgtgatgctattgctttatttgtaaccatta
taagctgcaataaacaagttaacaacaacaattgcattcattttatgtttcaggttcagggggaggtgtgggaggttttttagtcgactg
gggagagatctgaggaacccctagtgatggagttggccactccctctctgcgcgctcgctcgctcactgaggccgcccgggcaaagcccg
ggcgtcgggcgacctttggtcgcccggcctcagtgagcgagcgagcgcgcagagagggagtggccaac
>STAT3-DD amino acid sequence
(SEQ ID NO: 21)
MAQWNQLQQLDTRYLEQLHQLYSDSFPMELRQFLAPWIESQDWAYAASKESHATLVFHNLLGEIDQQYSRFLQESNVLYQHNLRRIKQFL
QSRYLEKPMEIARIVARCLWEESRLLQTAATAAQQGGQANHPTAAVVTEKQQMLEQHLQDVRKRVQDLEQKMKVVENLQDDFDFNYKTLK
SQGDMQDLNGNNQSVTRQKMQQLEQMLTALDQMRRSIVSELAGLLSAMEYVQKTLTDEELADWKRRQQIACIGGPPNICLDRLENWITSL
AESQLQTRQQIKKLEELQQKVSYKGDPIVQHRPMLEERIVELFRNLMKSAFVVERQPCMPMHPDRPLVIKTGVQFTTKVRLLVKFPELNY
QLKIKVCIDKDSGDVAALRGSRKFNILGTNTKVMNMEESNNGSLSAEFKHLTLREQRCGNGGRANCDASLIVTEELHLITFETEVYHQGL
KIDLETHSLPVVVISNICQMPNAWASILWYNMLTNNPKNVNFFTKPPIGTWDQVAEVLSWQFSSTTKRGLSIEQLTTLAEKLLGPGVNYS
GCQITWAKFCKENMAGKGFSFWVWLDNIIDLVKKYILALWNEGYIMGFISKERERAILSTKPPGTFLLRFSESSKEGGVTFTWVEKDISG
KTQIQSVEPYTKQQLNNMSFAEIIMGYKIMDATNILVSPLVYLYPDIPKEEAFGKYCRPESQEHPEADPGSAAPYLKTKFICVTPTTCSN
TIDLPMSPRTLDSLMQFGNNGEGAEPSAGGQFESLTFDMELTSECATSPMTRSRKKRSTRSRKKRSGAPMISLIAALAVDYVIGMENAMP
WNLPADLAWFKRNTLNKPVIMGRHTWESIGRPLPGRKNIILSSQPSTDDRVTWVKSVDEAIAACGDVPEIMVIGGGRVIEQFLPKAQKLY
LTHIDAEVEGDTHFPDYEPDDWESVFSEFHDADAQNSHSYCFEILERR*
>rAAV vector encoding STAT3 (TMP-regulated transcription factor STAT3)
(SEQ ID NO: 22)
ttggccactccctctctgcgcgctcgctcgctcactgaggccgggcgaccaaaggtcgcccgacgcccgggctttgcccgggcggcctca
gtgagcgagcgagcgcgcagagagggagtggccaactccatcactaggggttcctcagatctgaattcggtacctagttattaatagtaa
tcaattacggggtcattagttcatagcccatatatggagttccgcgttacataacttacggtaaatggcccgcctggctgaccgcccaac
gacccccgcccattgacgtcaataatgacgtatgttcccatagtaacgccaatagggactttccattgacgtcaatgggtggagtattta
cggtaaactgcccacttggcagtacatcaagtgtatcatatgccaagtacgccccctattgacgtcaatgacggtaaatggcccgcctgg
cattatgcccagtacatgaccttatgggactttcctacttggcagtacatctacgtattagtcatcgctattaccatggtcgaggtgagc
cccacgttctgcttcactctccccatctcccccccctccccacccccaattttgtatttatttattttttaattattttgtgcagcgatg
ggggcggggggggggggggggcgcgcgccaggcggggcggggcggggcgaggggcggggcggggcgaggcggagaggtgcggcggcagcc
aatcagagcggcgcgctccgaaagtttccttttatggcgaggcggcggcggcggcggccctataaaaagcgaagcgcgcggcgggcggga
gtcgctgcgcgctgccttcgccccgtgccccgctccgccgccgcctcgcgccgcccgccccggctctgactgaccgcgttactcccacag
gtgagcgggcgggacggcccttctcctccgggctgtaattagcgcttggtttaatgacggcttgtttcttttctgtggctgcgtgaaagc
cttgaggggctccgggagggccctttgtgcggggggagcggctcggggggtgcgtgcgtgtgtgtgtgcgtggggagcgccgcgtgcggc
tccgcgctgcccggcggctgtgagcgctgcgggcgcggcgcggggctttgtgcgctccgcagtgtgcgcgaggggagcgcggccgggggc
ggtgccccgcggtgcggggggggctgcgaggggaacaaaggctgcgtgcggggtgtgtgcgtgggggggtgagcagggggtgtgggcgcg
tcggtcgggctgcaaccccccctgcacccccctccccgagttgctgagcacggcccggcttcgggtgcggggctccgtacggggcgtggc
gcggggctcgccgtgccgggcggggggtggcggcaggtgggggtgccgggcggggcggggccgcctcgggccggggagggctcgggggag
gggcgcggcggcccccggagcgccggcggctgtcgaggcgcggcgagccgcagccattgccttttatggtaatcgtgcgagagggcgcag
ggacttcctttgtcccaaatctgtgcggagccgaaatctgggaggcgccgccgcaccccctctagcgggcgcggggcgaagcggtgcggc
gccggcaggaaggaaatgggcggggagggccttcgtgcgtcgccgcgccgccgtccccttctccctctccagcctcggggctgtccgcgg
ggggacggctgccttcgggggggacggggcagggcggggttcggcttctggcgtgtgaccggcggctctagagcctctgctaaccatgtt
catgccttcttctttttcctacagctcctgggcaacgtgctggttattgtgctgtctcatcattttggcaaagaattcctcgaagatcta
ggcaacgcgtctcgaggcggccgccgccaccatggctcaatggaaccaattgcaacaacttgatactcgatacctggagcaactccatca
gctgtactcagattcatttccgatggagctccggcagtttctggctccatggatagaatcacaggattgggcctacgcggcttcaaagga
aagtcacgcgaccctcgtatttcacaacctcctgggggaaattgatcagcaatatagtcggtttctgcaagaaagcaacgtcttgtacca
acataacttgaggaggattaagcaatttctccaaagccggtacttggagaagcctatggaaatcgctcgcattgtagcgaggtgcctgtg
ggaggagagtcgccttctgcagaccgccgccacagccgctcaacaaggtgggcaggctaatcatcccaccgctgctgtggtcaccgaaaa
gcagcagatgctcgagcaacaccttcaggacgtcagaaagagggttcaggatctggaacaaaagatgaaagtcgtggagaatttgcagga
tgattttgattttaactacaagaccttgaaatcccaaggcgatatgcaagatcttaatggaaacaaccagtcagtgaccaggcagaagat
gcaacaacttgaacaaatgttgactgccctcgatcaaatgagacgcagtatagtaagcgagttggccggtcttctctcagcgatggagta
cgtacagaaaacgctgactgatgaagagctggctgactggaagcggcggcagcagatagcttgcatcggtgggcccccaaatatatgcct
ggatcgcctggagaactggataacgagtctggccgagagtcagctgcagactcgccagcagatcaaaaaactggaagaactccagcaaaa
agtttcatacaagggagatccgattgtacagcatcgccccatgctggaagagcgaattgttgaactctttagaaatttgatgaagagcgc
gttcgttgtagagcggcagccgtgtatgcctatgcacccggaccggccacttgtgataaagaccggagttcagtttacaacaaaggtccg
actgcttgtcaaatttccggaattgaactatcaattgaagatcaaggtttgcatcgataaggattctggcgatgttgcggcgttgcgcgg
cagccgcaagtttaacatacttggcactaataccaaggttatgaacatggaggagtccaacaacggctcactcagtgctgaattcaagca
ccttacgctgagagaacagagatgcggtaacgggggacgcgccaactgtgatgcctcacttattgtcactgaagagcttcacctgataac
gttcgaaactgaggtttaccatcaaggcctgaaaatagatctggagactcattctctgcccgttgtcgttatttctaacatttgccagat
gcccaatgcatgggcgagcattctgtggtataatatgctgacaaacaatccgaagaacgtcaacttcttcacgaagccgccgatcggtac
ctgggatcaggtggcggaagtccttagctggcagttcagctccaccaccaagaggggtttgtcaatagagcaattgactacgttggctga
gaagctcctgggacctggagtaaattacagtgggtgtcagatcacatgggctaaattttgcaaggaaaacatggcaggtaagggtttttc
cttttgggtctggcttgataacataatagatctcgtcaaaaagtacatactcgcgctgtggaacgagggttacataatgggttttatatc
taaagaaagggagagagcgattttgtccactaaaccaccgggcacgtttcttctccgattttccgagtcaagcaaagaaggtggcgtaac
atttacctgggtcgaaaaggacattagtggaaagacgcaaattcaaagtgtggaaccttatacgaaacagcaactgaataacatgtcctt
tgcggagataataatggggtacaaaattatggatgctactaatatactcgttagccctctggtctacctttacccagacatccccaagga
ggaggcgttcggaaagtattgccgaccggaatcccaagagcatcccgaagcggaccccggtagtgccgcaccctaccttaaaactaagtt
catttgcgtcaccccaacgacgtgcagcaacacaatagacttgccgatgagtccccgaacgctcgatagtctcatgcagttcggaaacaa
tggcgagggagctgagccctccgccggcggacaatttgagagcctcacctttgacatggagcttacatcagagtgcgcgacaagcccgat
gaccagatctcgcaagaagcgcagcaccagatctcgcaagaagcgcagcggggcccccatgatatctctgatcgctgctcttgctgtgga
ctacgtgattggtatggagaatgctatgccctggaatctgcctgcagacctggcatggtttaagaggaacacccttaataaacctgtaat
catgggacgacatacatgggagagtatcggcaggccattgcccgggaggaagaatataattctgagttcccagccttctactgatgatag
ggtaacttgggtcaagagcgtcgacgaggccatcgccgcttgcggggatgtgcctgaaatcatggttatcgggggcggacgggtgattga
gcaatttctgcccaaggcacagaagctgtatctgacccatatagacgccgaggtggagggagatacacactttcccgactatgagcccga
tgattgggagagtgtctttagcgaatttcatgacgccgacgcacagaattcccatagctactgcttcgaaattctcgaacggcgctaggc
ggccgcgcggatccagacatgataagatacattgatgagtttggacaaaccacaactagaatgcagtgaaaaaaatgctttatttgtgaa
atttgtgatgctattgctttatttgtaaccattataagctgcaataaacaagttaacaacaacaattgcattcattttatgtttcaggtt
cagggggaggtgtgggaggttttttagtcgactggggagagatctgaggaacccctagtgatggagttggccactccctctctgcgcgct
cgctcgctcactgaggccgcccgggcaaagcccgggcgtcgggcgacctttggtcgcccggcctcagtgagcgagcgagcgcgcagagag
ggagtggccaac
>STAT3 Y705F-DD amino acid sequence
(SEQ ID NO: 23)
MAQWNQLQQLDTRYLEQLHQLYSDSFPMELRQFLAPWIESQDWAYAASKESHATLVFHNLLGEIDQQYSRFLQESNVLYQHNLRRIKQFL
QSRYLEKPMEIARIVARCLWEESRLLQTAATAAQQGGQANHPTAAVVTEKQQMLEQHLQDVRKRVQDLEQKMKVVENLQDDFDFNYKTLK
SQGDMQDLNGNNQSVTRQKMQQLEQMLTALDQMRRSIVSELAGLLSAMEYVQKTLTDEELADWKRRQQIACIGGPPNICLDRLENWITSL
AESQLQTRQQIKKLEELQQKVSYKGDPIVQHRPMLEERIVELFRNLMKSAFVVERQPCMPMHPDRPLVIKTGVQFTTKVRLLVKFPELNY
QLKIKVCIDKDSGDVAALRGSRKFNILGTNTKVMNMEESNNGSLSAEFKHLTLREQRCGNGGRANCDASLIVTEELHLITFETEVYHQGL
KIDLETHSLPVVVISNICQMPNAWASILWYNMLTNNPKNVNFFTKPPIGTWDQVAEVLSWQFSSTTKRGLSIEQLTTLAEKLLGPGVNYS
GCQITWAKFCKENMAGKGFSFWVWLDNIIDLVKKYILALWNEGYIMGFISKERERAILSTKPPGTFLLRFSESSKEGGVTFTWVEKDISG
KTQIQSVEPYTKQQLNNMSFAEIIMGYKIMDATNILVSPLVYLYPDIPKEEAFGKYCRPESQEHPEADPGSAAPFLKTKFICVTPTTCSN
TIDLPMSPRTLDSLMQFGNNGEGAEPSAGGQFESLTFDMELTSECATSPMTRSRKKRSTRSRKKRSGAPMISLIAALAVDYVIGMENAMP
WNLPADLAWFKRNTLNKPVIMGRHTWESIGRPLPGRKNIILSSQPSTDDRVTWVKSVDEAIAACGDVPEIMVIGGGRVIEQFLPKAQKLY
LTHIDAEVEGDTHFPDYEPDDWESVFSEFHDADAQNSHSYCFEILERR*
>rAAV vector encoding STAT3 Y705F (TMP-regulated phosphomemetic of the transcription
factor STAT3 at amino acid 705)
(SEQ ID NO: 24)
ttggccactccctctctgcgcgctcgctcgctcactgaggccgggcgaccaaaggtcgcccgacgcccgggctttgcccgggcggcctca
gtgagcgagcgagcgcgcagagagggagtggccaactccatcactaggggttcctcagatctgaattcggtacctagttattaatagtaa
tcaattacggggtcattagttcatagcccatatatggagttccgcgttacataacttacggtaaatggcccgcctggctgaccgcccaac
gacccccgcccattgacgtcaataatgacgtatgttcccatagtaacgccaatagggactttccattgacgtcaatgggtggagtattta
cggtaaactgcccacttggcagtacatcaagtgtatcatatgccaagtacgccccctattgacgtcaatgacggtaaatggcccgcctgg
cattatgcccagtacatgaccttatgggactttcctacttggcagtacatctacgtattagtcatcgctattaccatggtcgaggtgagc
cccacgttctgcttcactctccccatctcccccccctccccacccccaattttgtatttatttattttttaattattttgtgcagcgatg
ggggcggggggggggggggggcgcgcgccaggcggggcggggcggggcgaggggcggggcggggcgaggcggagaggtgcggcggcagcc
aatcagagcggcgcgctccgaaagtttccttttatggcgaggcggcggcggcggcggccctataaaaagcgaagcgcgcggcgggcggga
gtcgctgcgcgctgccttcgccccgtgccccgctccgccgccgcctcgcgccgcccgccccggctctgactgaccgcgttactcccacag
gtgagcgggcgggacggcccttctcctccgggctgtaattagcgcttggtttaatgacggcttgtttcttttctgtggctgcgtgaaagc
cttgaggggctccgggagggccctttgtgcggggggagcggctcggggggtgcgtgcgtgtgtgtgtgcgtggggagcgccgcgtgcggc
tccgcgctgcccggcggctgtgagcgctgcgggcgcggcgcggggctttgtgcgctccgcagtgtgcgcgaggggagcgcggccgggggc
ggtgccccgcggtgcggggggggctgcgaggggaacaaaggctgcgtgcggggtgtgtgcgtgggggggtgagcagggggtgtgggcgcg
tcggtcgggctgcaaccccccctgcacccccctccccgagttgctgagcacggcccggcttcgggtgcggggctccgtacggggcgtggc
gcggggctcgccgtgccgggcggggggtggcggcaggtgggggtgccgggcggggcggggccgcctcgggccggggagggctcgggggag
gggcgcggcggcccccggagcgccggcggctgtcgaggcgcggcgagccgcagccattgccttttatggtaatcgtgcgagagggcgcag
ggacttcctttgtcccaaatctgtgcggagccgaaatctgggaggcgccgccgcaccccctctagcgggcgcggggcgaagcggtgcggc
gccggcaggaaggaaatgggcggggagggccttcgtgcgtcgccgcgccgccgtccccttctccctctccagcctcggggctgtccgcgg
ggggacggctgccttcgggggggacggggcagggcggggttcggcttctggcgtgtgaccggcggctctagagcctctgctaaccatgtt
catgccttcttctttttcctacagctcctgggcaacgtgctggttattgtgctgtctcatcattttggcaaagaattcctcgaagatcta
ggcaacgcgtctcgaggcggccgccgccaccatggcccagtggaatcaacttcaacaacttgatacccgctacctggaacaacttcatca
actctacagtgatagctttcctatggagctcagacagttcctcgcgccgtggattgaaagccaggactgggcgtatgctgcttcaaagga
gagtcatgcaactctcgtattccataatctgctcggagagatcgatcaacaatacagcaggtttctgcaggaatctaacgtgctttatca
acacaatctgaggcggattaaacaattcctgcaatctcgatatcttgaaaaacccatggagattgcccggatcgtcgcacgctgtctgtg
ggaagagagtcgactgttgcagacggcagctactgctgcccagcagggcggacaggcgaatcatccgactgccgctgtagtgacggaaaa
acaacaaatgctggagcaacatctgcaagacgtcagaaaacgagtacaggacctcgaacagaagatgaaagtggtcgaaaacttgcaaga
cgacttcgattttaattataaaacgctgaaatcccagggggatatgcaagaccttaacggcaataatcagtctgttaccagacagaaaat
gcagcagctcgagcaaatgctgacggctcttgaccagatgagaagaagcatagtctcagaattggcaggcctcctctctgccatggagta
cgtacagaagacgttgacagatgaggaactcgctgattggaagagacgccaacagattgcttgcatagggggtccacctaatatatgcct
cgaccggttggaaaactggataacatctttggctgaatctcagctccaaacacgccagcaaataaaaaagcttgaggagttgcagcagaa
ggttagttacaagggagatcccattgtgcagcataggccaatgctggaagagaggattgttgagctgtttaggaatctgatgaaaagtgc
cttcgtagtggagagacagccatgcatgccaatgcatcctgacagaccactggttataaagactggagttcaatttacaacaaaagttag
actcctggtcaagttcccggagctcaactatcaactgaagataaaagtctgtattgacaaggatagtggcgatgttgcagccttgagggg
tagccgaaaatttaacattctcggaacgaacacaaaagtgatgaacatggaggaatcaaataatggttctctctccgcagagtttaagca
ccttactctgcgcgaacaacgctgcggaaacggcggccgggccaattgcgatgccagccttatagtgacggaagagctgcatctgataac
tttcgagacggaagtctatcatcaaggcctcaaaattgacttggagactcactccttgcccgtagtggttatttcaaatatatgccaaat
gcctaatgcatgggcatcaatcctttggtacaatatgcttactaataacccaaagaacgttaatttcttcactaaacctccaattggtac
ttgggaccaagttgccgaggtattgagttggcagttttcctcaacaactaagagagggttgagcatagaacagctgaccacactggcgga
gaaacttctgggacccggcgtcaactattctggatgtcagattacctgggccaaattctgcaaagaaaatatggctggcaagggtttcag
cttctgggtatggcttgacaatataattgacctcgtaaaaaagtacatcctcgctctttggaatgaaggttatattatgggctttataag
taaagagcgagagcgggctatcttgtcaacgaagcccccgggtacgttcctgttgcgattctcagagagctctaaggagggtggcgtgac
tttcacgtgggtagagaaagacatctctggtaagactcaaatccaatcagttgaaccgtacacaaagcaacaactgaataacatgtcctt
cgcagaaattatcatggggtacaagattatggacgcgaccaacatcctggtcagtccgctcgtttatctctatcccgatatacctaaaga
agaagcatttggaaagtactgccggccagaatcacaagagcatcctgaggctgatcctggttccgctgcaccgttcttgaaaacgaaatt
catatgtgtcactcctacaacctgttcaaacacgatagaccttccaatgtctcctaggacgttggacagccttatgcagtttggtaataa
tggggagggggccgagccgtccgcggggggtcagtttgagagccttacattcgatatggagctgacctctgagtgtgcaacttctccgat
gaccagatctcgcaagaagcgcagcaccagatctcgcaagaagcgcagcggggcccccatgatatctctgatcgctgctcttgctgtgga
ctacgtgattggtatggagaatgctatgccctggaatctgcctgcagacctggcatggtttaagaggaacacccttaataaacctgtaat
catgggacgacatacatgggagagtatcggcaggccattgcccgggaggaagaatataattctgagttcccagccttctactgatgatag
ggtaacttgggtcaagagcgtcgacgaggccatcgccgcttgcggggatgtgcctgaaatcatggttatcgggggcggacgggtgattga
gcaatttctgcccaaggcacagaagctgtatctgacccatatagacgccgaggtggagggagatacacactttcccgactatgagcccga
tgattgggagagtgtctttagcgaatttcatgacgccgacgcacagaattcccatagctactgcttcgaaattctcgaacggcgctaggc
ggccgcgcggatccagacatgataagatacattgatgagtttggacaaaccacaactagaatgcagtgaaaaaaatgctttatttgtgaa
atttgtgatgctattgctttatttgtaaccattataagctgcaataaacaagttaacaacaacaattgcattcattttatgtttcaggtt
cagggggaggtgtgggaggttttttagtcgactggggagagatctgaggaacccctagtgatggagttggccactccctctctgcgcgct
cgctcgctcactgaggccgcccgggcaaagcccgggcgtcgggcgacctttggtcgcccggcctcagtgagcgagcgagcgcgcagagag
ggagtggccaac
>STAT3 S727E-DD amino acid sequence
(SEQ ID NO: 25)
MAQWNQLQQLDTRYLEQLHQLYSDSFPMELRQFLAPWIESQDWAYAASKESHATLVFHNLLGEIDQQYSRFLQESNVLYQHNLRRIKQFL
QSRYLEKPMEIARIVARCLWEESRLLQTAATAAQQGGQANHPTAAVVTEKQQMLEQHLQDVRKRVQDLEQKMKVVENLQDDFDFNYKTLK
SQGDMQDLNGNNQSVTRQKMQQLEQMLTALDQMRRSIVSELAGLLSAMEYVQKTLTDEELADWKRRQQIACIGGPPNICLDRLENWITSL
AESQLQTRQQIKKLEELQQKVSYKGDPIVQHRPMLEERIVELFRNLMKSAFVVERQPCMPMHPDRPLVIKTGVQFTTKVRLLVKFPELNY
QLKIKVCIDKDSGDVAALRGSRKFNILGTNTKVMNMEESNNGSLSAEFKHLTLREQRCGNGGRANCDASLIVTEELHLITFETEVYHQGL
KIDLETHSLPVVVISNICQMPNAWASILWYNMLTNNPKNVNFFTKPPIGTWDQVAEVLSWQFSSTTKRGLSIEQLTTLAEKLLGPGVNYS
GCQITWAKFCKENMAGKGFSFWVWLDNIIDLVKKYILALWNEGYIMGFISKERERAILSTKPPGTFLLRFSESSKEGGVTFTWVEKDISG
KTQIQSVEPYTKQQLNNMSFAEIIMGYKIMDATNILVSPLVYLYPDIPKEEAFGKYCRPESQEHPEADPGSAAPYLKTKFICVTPTTCSN
TIDLPMEPRTLDSLMQFGNNGEGAEPSAGGQFESLTFDMELTSECATSPMTRSRKKRSTRSRKKRSGAPMISLIAALAVDYVIGMENAMP
WNLPADLAWFKRNTLNKPVIMGRHTWESIGRPLPGRKNIILSSQPSTDDRVTWVKSVDEAIAACGDVPEIMVIGGGRVIEQFLPKAQKLY
LTHIDAEVEGDTHFPDYEPDDWESVFSEFHDADAQNSHSYCFEILERR*
>rAAV vector encoding STAT3 S727E (TMP-regulated phosphomemetic of the transcription
factor STAT3 at amino acid 727)
(SEQ ID NO: 26)
ttggccactccctctctgcgcgctcgctcgctcactgaggccgggcgaccaaaggtcgcccgacgcccgggctttgcccgggcggcctca
gtgagcgagcgagcgcgcagagagggagtggccaactccatcactaggggttcctcagatctgaattcggtacctagttattaatagtaa
tcaattacggggtcattagttcatagcccatatatggagttccgcgttacataacttacggtaaatggcccgcctggctgaccgcccaac
gacccccgcccattgacgtcaataatgacgtatgttcccatagtaacgccaatagggactttccattgacgtcaatgggtggagtattta
cggtaaactgcccacttggcagtacatcaagtgtatcatatgccaagtacgccccctattgacgtcaatgacggtaaatggcccgcctgg
cattatgcccagtacatgaccttatgggactttcctacttggcagtacatctacgtattagtcatcgctattaccatggtcgaggtgagc
cccacgttctgcttcactctccccatctcccccccctccccacccccaattttgtatttatttattttttaattattttgtgcagcgatg
ggggcggggggggggggggggcgcgcgccaggcggggcggggcggggcgaggggcggggcggggcgaggcggagaggtgcggcggcagcc
aatcagagcggcgcgctccgaaagtttccttttatggcgaggcggcggcggcggcggccctataaaaagcgaagcgcgcggcgggcggga
gtcgctgcgcgctgccttcgccccgtgccccgctccgccgccgcctcgcgccgcccgccccggctctgactgaccgcgttactcccacag
gtgagcgggcgggacggcccttctcctccgggctgtaattagcgcttggtttaatgacggcttgtttcttttctgtggctgcgtgaaagc
cttgaggggctccgggagggccctttgtgcggggggagcggctcggggggtgcgtgcgtgtgtgtgtgcgtggggagcgccgcgtgcggc
tccgcgctgcccggcggctgtgagcgctgcgggcgcggcgcggggctttgtgcgctccgcagtgtgcgcgaggggagcgcggccgggggc
ggtgccccgcggtgcggggggggctgcgaggggaacaaaggctgcgtgcggggtgtgtgcgtgggggggtgagcagggggtgtgggcgcg
tcggtcgggctgcaaccccccctgcacccccctccccgagttgctgagcacggcccggcttcgggtgcggggctccgtacggggcgtggc
gcggggctcgccgtgccgggcggggggtggcggcaggtgggggtgccgggcggggcggggccgcctcgggccggggagggctcgggggag
gggcgcggcggcccccggagcgccggcggctgtcgaggcgcggcgagccgcagccattgccttttatggtaatcgtgcgagagggcgcag
ggacttcctttgtcccaaatctgtgcggagccgaaatctgggaggcgccgccgcaccccctctagcgggcgcggggcgaagcggtgcggc
gccggcaggaaggaaatgggcggggagggccttcgtgcgtcgccgcgccgccgtccccttctccctctccagcctcggggctgtccgcgg
ggggacggctgccttcgggggggacggggcagggcggggttcggcttctggcgtgtgaccggcggctctagagcctctgctaaccatgtt
catgccttcttctttttcctacagctcctgggcaacgtgctggttattgtgctgtctcatcattttggcaaagaattcctcgaagatcta
ggcaacgcgtctcgaggcggccgccgccaccatggctcaatggaaccagctccaacagctcgacaccaggtacctggagcaattgcatca
actctatagtgatagcttccccatggaactgagacaattccttgcaccatggatagagtcacaggattgggcgtacgctgcaagcaaaga
atctcatgcaaccctcgtatttcataatctcctcggtgagatcgaccagcaatactcacggttccttcaggagtctaatgtgctttatca
acacaaccttcgccggattaagcagttcttgcagagccgatatctcgagaagcctatggaaatagctaggatcgttgcaaggtgcttgtg
ggaagaaagtagacttctccaaactgcagcaacggctgcgcagcagggtggccaggccaaccacccgacagccgccgtggtaacggaaaa
acagcagatgcttgaacaacaccttcaggacgtccgcaaaagagtacaggacctggaacaaaaaatgaaggtcgtcgaaaaccttcagga
tgacttcgattttaactataaaaccctcaaatctcagggagatatgcaagacctcaacggtaataatcagagcgtcactcgacagaagat
gcagcaacttgagcaaatgcttacggcactggatcagatgcgaaggtccattgtctccgaacttgccgggctcctgtcagccatggagta
tgtacagaaaactttgaccgacgaggagttggcagactggaagaggaggcaacagatagcctgcattggcgggccccctaacatttgcct
tgacaggttggaaaactggattacatctctggcggagagtcaactgcagacacggcagcaaataaaaaaactggaagagctccagcagaa
ggtgagttacaagggggacccaattgtgcagcatcgcccaatgctggaagaacggattgttgaattgtttaggaacctcatgaaatccgc
gtttgtcgtagagagacagccctgtatgccgatgcatcctgatcgacccctcgtgatcaaaactggtgtacaattcacaacaaaagtgag
gttgctggtcaagttccctgaactcaactaccaactgaagattaaagtgtgcattgacaaggattctggagacgtggcggcacttcgcgg
ttcaagaaagtttaacatactcggcactaatacaaaagttatgaatatggaggagtccaacaacgggtcactgtccgccgaatttaagca
cttgacgcttcgcgagcaacgatgtggcaatgggggcagagcaaattgcgacgcttctttgattgtcactgaggaacttcacctgattac
cttcgaaaccgaggtataccaccaagggctgaaaattgacttggaaacccatagtctccccgtggtggtcattagtaacatttgccagat
gcctaatgcttgggcaagcatcctttggtacaacatgcttacaaacaatccgaagaacgtcaacttctttaccaagccgcccattggaac
atgggatcaagtggcggaagtcctcagttggcagttttccagtaccacgaagcgaggactttctatcgagcaactgacgacgctggcgga
aaaactcttgggacctggagttaattactccgggtgtcagataacatgggccaaattctgcaaggagaacatggcggggaaaggtttcag
cttttgggtttggttggataacataattgatctcgtaaagaaatacatactggccctctggaatgaaggttatatcatgggcttcattag
taaagaacgggagagggccatacttagcactaagcctccaggaacgtttcttctcagattttctgagtcttcaaaggagggaggcgtgac
cttcacctgggttgaaaaagatatctcaggaaagacacagattcagtccgtcgaaccatatacgaaacagcaactcaataacatgagttt
cgcagaaataatcatgggatataagattatggacgcaactaatatactggtgagtccactcgtttatctctaccccgatatcccgaagga
ggaagcattcggaaaatactgcagacctgaatcccaggaacatccagaggccgacccaggctctgccgctccatatttgaaaactaaatt
tatctgcgtaacacctacaacttgcagcaatacgatcgatttgcccatggaaccacgcaccctcgatagccttatgcaattcgggaataa
tggggaaggagcagaaccctctgcaggaggacaatttgagagcctcacattcgatatggagttgacgtcagaatgcgcaaccagtccaat
gaccagatctcgcaagaagcgcagcaccagatctcgcaagaagcgcagcggggcccccatgatatctctgatcgctgctcttgctgtgga
ctacgtgattggtatggagaatgctatgccctggaatctgcctgcagacctggcatggtttaagaggaacacccttaataaacctgtaat
catgggacgacatacatgggagagtatcggcaggccattgcccgggaggaagaatataattctgagttcccagccttctactgatgatag
ggtaacttgggtcaagagcgtcgacgaggccatcgccgcttgcggggatgtgcctgaaatcatggttatcgggggcggacgggtgattga
gcaatttctgcccaaggcacagaagctgtatctgacccatatagacgccgaggtggagggagatacacactttcccgactatgagcccga
tgattgggagagtgtctttagcgaatttcatgacgccgacgcacagaattcccatagctactgcttcgaaattctcgaacggcgctaggc
ggccgcgcggatccagacatgataagatacattgatgagtttggacaaaccacaactagaatgcagtgaaaaaaatgctttatttgtgaa
atttgtgatgctattgctttatttgtaaccattataagctgcaataaacaagttaacaacaacaattgcattcattttatgtttcaggtt
cagggggaggtgtgggaggttttttagtcgactggggagagatctgaggaacccctagtgatggagttggccactccctctctgcgcgct
cgctcgctcactgaggccgcccgggcaaagcccgggcgtcgggcgacctttggtcgcccggcctcagtgagcgagcgagcgcgcagagag
ggagtggccaac
>VEGFR2 IgG 1-3-DD amino acid sequence
(SEQ ID NO: 27)
MQSKVLLAVALWLCVETRAASVGLPSVSLDLPRLSIQKDILTIKANTTLQITCRGQRDLDWLWPNNQSGSEQRVEVTECSDGLFCKTLTI
PKVIGNDTGAYKCFYRETDLASVIYVYVQDYRSPFIASVSDQHGVVYITENKNKTVVIPCLGSISNLNVSLCARYPEKRFVPDGNRISWD
SKKGFTIPSYMISYAGMVFCEAKINDESYQSIMYIVVVVGYRIYDVVLSPSHGIELSVGEKLVLNCTARTELNVGIDFNWEYPSSKHQHK
KLVNRDLKTQSGSEMKKFLSTLTIDGVTRSDQGLYTCAASSGLMTKKNSTTRSRKKRSTRSRKKRSGAPMISLIAALAVDYVIGMENAMP
WNLPADLAWFKRNTLNKPVIMGRHTWESIGRPLPGRKNIILSSQPSTDDRVTWVKSVDEAIAACGDVPEIMVIGGGRVIEQFLPKAQKLY
LTHIDAEVEGDTHFPDYEPDDWESVFSEFHDADAQNSHSYCFEILERR*
>rAAV vector encoding VEGFR2 IgG 1-3 (TMP-regulated IgG loops 1-3 of VEGFR2)
(SEQ ID NO: 28)
ttggccactccctctctgcgcgctcgctcgctcactgaggccgggcgaccaaaggtcgcccgacgcccgggctttgcccgggcggcctca
gtgagcgagcgagcgcgcagagagggagtggccaactccatcactaggggttcctcagatctgaattcggtacctagttattaatagtaa
tcaattacggggtcattagttcatagcccatatatggagttccgcgttacataacttacggtaaatggcccgcctggctgaccgcccaac
gacccccgcccattgacgtcaataatgacgtatgttcccatagtaacgccaatagggactttccattgacgtcaatgggtggagtattta
cggtaaactgcccacttggcagtacatcaagtgtatcatatgccaagtacgccccctattgacgtcaatgacggtaaatggcccgcctgg
cattatgcccagtacatgaccttatgggactttcctacttggcagtacatctacgtattagtcatcgctattaccatggtcgaggtgagc
cccacgttctgcttcactctccccatctcccccccctccccacccccaattttgtatttatttattttttaattattttgtgcagcgatg
ggggcggggggggggggggggcgcgcgccaggcggggcggggcggggcgaggggcggggcggggcgaggcggagaggtgcggcggcagcc
aatcagagcggcgcgctccgaaagtttccttttatggcgaggcggcggcggcggcggccctataaaaagcgaagcgcgcggcgggcggga
gtcgctgcgcgctgccttcgccccgtgccccgctccgccgccgcctcgcgccgcccgccccggctctgactgaccgcgttactcccacag
gtgagcgggcgggacggcccttctcctccgggctgtaattagcgcttggtttaatgacggcttgtttcttttctgtggctgcgtgaaagc
cttgaggggctccgggagggccctttgtgcggggggagcggctcggggggtgcgtgcgtgtgtgtgtgcgtggggagcgccgcgtgcggc
tccgcgctgcccggcggctgtgagcgctgcgggcgcggcgcggggctttgtgcgctccgcagtgtgcgcgaggggagcgcggccgggggc
ggtgccccgcggtgcggggggggctgcgaggggaacaaaggctgcgtgcggggtgtgtgcgtgggggggtgagcagggggtgtgggcgcg
tcggtcgggctgcaaccccccctgcacccccctccccgagttgctgagcacggcccggcttcgggtgcggggctccgtacggggcgtggc
gcggggctcgccgtgccgggcggggggtggcggcaggtgggggtgccgggcggggcggggccgcctcgggccggggagggctcgggggag
gggcgcggcggcccccggagcgccggcggctgtcgaggcgcggcgagccgcagccattgccttttatggtaatcgtgcgagagggcgcag
ggacttcctttgtcccaaatctgtgcggagccgaaatctgggaggcgccgccgcaccccctctagcgggcgcggggcgaagcggtgcggc
gccggcaggaaggaaatgggcggggagggccttcgtgcgtcgccgcgccgccgtccccttctccctctccagcctcggggctgtccgcgg
ggggacggctgccttcgggggggacggggcagggcggggttcggcttctggcgtgtgaccggcggctctagagcctctgctaaccatgtt
catgccttcttctttttcctacagctcctgggcaacgtgctggttattgtgctgtctcatcattttggcaaagaattcctcgaagatcta
ggcaacgcgtctcgaggcggccgccgccaccatgcaatcaaaggtgttgctggcggtggccctttggttgtgtgtcgagacccgagcggc
ctccgtgggtcttccctccgtttctctcgatctccctcggttgagcatccaaaaagacatccttaccataaaagcaaacactaccttgca
gatcacatgcagaggtcagagagatctcgattggttgtggcccaataatcagagcgggtccgaacagcgggttgaggtgaccgagtgctc
agatggacttttctgtaagaccctcacgatacccaaagttataggcaacgatactggtgcctacaagtgcttctaccgagaaacggattt
ggcctcagttatatacgtttatgtgcaagattaccgctccccattcatagcgtcagtcagcgaccaacacggcgttgtctacattacgga
aaacaagaataagacggtcgtgataccgtgtctcggaagcatctcaaatctgaatgtctccctgtgcgctcgctaccctgagaaaaggtt
tgttcctgacggtaacagaatatcctgggatagtaaaaaaggttttacgattccctcctatatgattagttatgctggcatggtgttttg
cgaggctaaaatcaacgatgagtcctaccagtcaataatgtatattgtggtggttgtgggctacaggatctacgatgtggttttgtctcc
cagccatggaatcgagttgtctgtaggggaaaaactggtgcttaactgcacagcgagaaccgaactcaacgttggaatagacttcaactg
ggagtacccgagttccaaacaccaacacaaaaaactcgtaaacagggatctgaagacccagtcagggtctgagatgaaaaaatttctttc
tactctgacaatagatggagtcacccgctccgatcaagggctctacacttgtgcggcttcttcagggctgatgaccaaaaaaaattcaac
taccagatctcgcaagaagcgcagcaccagatctcgcaagaagcgcagcggggcccccatgatatctctgatcgctgctcttgctgtgga
ctacgtgattggtatggagaatgctatgccctggaatctgcctgcagacctggcatggtttaagaggaacacccttaataaacctgtaat
catgggacgacatacatgggagagtatcggcaggccattgcccgggaggaagaatataattctgagttcccagccttctactgatgatag
ggtaacttgggtcaagagcgtcgacgaggccatcgccgcttgcggggatgtgcctgaaatcatggttatcgggggcggacgggtgattga
gcaatttctgcccaaggcacagaagctgtatctgacccatatagacgccgaggtggagggagatacacactttcccgactatgagcccga
tgattgggagagtgtctttagcgaatttcatgacgccgacgcacagaattcccatagctactgcttcgaaattctcgaacggcgctaggc
ggccgcgcggatccagacatgataagatacattgatgagtttggacaaaccacaactagaatgcagtgaaaaaaatgctttatttgtgaa
atttgtgatgctattgctttatttgtaaccattataagctgcaataaacaagttaacaacaacaattgcattcattttatgtttcaggtt
cagggggaggtgtgggaggttttttagtcgactggggagagatctgaggaacccctagtgatggagttggccactccctctctgcgcgct
cgctcgctcactgaggccgcccgggcaaagcccgggcgtcgggcgacctttggtcgcccggcctcagtgagcgagcgagcgcgcagagag
ggagtggccaac
>VEGFR2 IgG 1-7-DD amino acid sequence
(SEQ ID NO: 29)
MQSKVLLAVALWLCVETRAASVGLPSVSLDLPRLSIQKDILTIKANTTLQITCRGQRDLDWLWPNNQSGSEQRVEVTECSDGLFCKTLTI
PKVIGNDTGAYKCFYRETDLASVIYVYVQDYRSPFIASVSDQHGVVYITENKNKTVVIPCLGSISNLNVSLCARYPEKRFVPDGNRISWD
SKKGFTIPSYMISYAGMVFCEAKINDESYQSIMYIVVVVGYRIYDVVLSPSHGIELSVGEKLVLNCTARTELNVGIDFNWEYPSSKHQHK
KLVNRDLKTQSGSEMKKFLSTLTIDGVTRSDQGLYTCAASSGLMTKKNSTFVRVHEKPFVAFGSGMESLVEATVGERVRIPAKYLGYPPP
EIKWYKNGIPLESNHTIKAGHVLTIMEVSERDTGNYTVILTNPISKEKQSHVVSLVVYVPPQIGEKSLISPVDSYQYGTTQTLTCTVYAI
PPPHHIHWYWQLEEECANEPSQAVSVTNPYPCEEWRSVEDFQGGNKIEVNKNQFALIEGKNKTVSTLVIQAANVSALYKCEAVNKVGRGE
RVISFHVTRGPEITLQPDMQPTEQESVSLWCTADRSTFENLTWYKLGPQPLPIHVGELPTPVCKNLDTLWKLNATMFSNSTNDILIMELK
NASLQDQGDYVCLAQDRKTKKRHCVVRQLTVLERVAPTITGNLENQTTSIGESIEVSCTASGNPPPQIMWFKDNETLVEDSGIVLKDGNR
NLTIRRVRKEDEGLYTCQACSVLGCAKVEAFFITRSRKKRSTRSRKKRSGAPMISLIAALAVDYVIGMENAMPWNLPADLAWFKRNTLNK
PVIMGRHTWESIGRPLPGRKNIILSSQPSTDDRVTWVKSVDEAIAACGDVPEIMVIGGGRVIEQFLPKAQKLYLTHIDAEVEGDTHFPDY
EPDDWESVFSEFHDADAQNSHSYCFEILERR*
>rAAV vector encoding VEGFR2 IgG 1-7 (TMP-regulated IgG loops 1-7 of VEGFR2)
(SEQ ID NO: 30
ttggccactccctctctgcgcgctcgctcgctcactgaggccgggcgaccaaaggtcgcccgacgcccgggctttgcccgggcggcctca
gtgagcgagcgagcgcgcagagagggagtggccaactccatcactaggggttcctcagatctgaattcggtacctagttattaatagtaa
tcaattacggggtcattagttcatagcccatatatggagttccgcgttacataacttacggtaaatggcccgcctggctgaccgcccaac
gacccccgcccattgacgtcaataatgacgtatgttcccatagtaacgccaatagggactttccattgacgtcaatgggtggagtattta
cggtaaactgcccacttggcagtacatcaagtgtatcatatgccaagtacgccccctattgacgtcaatgacggtaaatggcccgcctgg
cattatgcccagtacatgaccttatgggactttcctacttggcagtacatctacgtattagtcatcgctattaccatggtcgaggtgagc
cccacgttctgcttcactctccccatctcccccccctccccacccccaattttgtatttatttattttttaattattttgtgcagcgatg
ggggcggggggggggggggggcgcgcgccaggcggggcggggcggggcgaggggcggggcggggcgaggcggagaggtgcggcggcagcc
aatcagagcggcgcgctccgaaagtttccttttatggcgaggcggcggcggcggcggccctataaaaagcgaagcgcgcggcgggcggga
gtcgctgcgcgctgccttcgccccgtgccccgctccgccgccgcctcgcgccgcccgccccggctctgactgaccgcgttactcccacag
gtgagcgggcgggacggcccttctcctccgggctgtaattagcgcttggtttaatgacggcttgtttcttttctgtggctgcgtgaaagc
cttgaggggctccgggagggccctttgtgcggggggagcggctcggggggtgcgtgcgtgtgtgtgtgcgtggggagcgccgcgtgcggc
tccgcgctgcccggcggctgtgagcgctgcgggcgcggcgcggggctttgtgcgctccgcagtgtgcgcgaggggagcgcggccgggggc
ggtgccccgcggtgcggggggggctgcgaggggaacaaaggctgcgtgcggggtgtgtgcgtgggggggtgagcagggggtgtgggcgcg
tcggtcgggctgcaaccccccctgcacccccctccccgagttgctgagcacggcccggcttcgggtgcggggctccgtacggggcgtggc
gcggggctcgccgtgccgggcggggggtggcggcaggtgggggtgccgggcggggcggggccgcctcgggccggggagggctcgggggag
gggcgcggcggcccccggagcgccggcggctgtcgaggcgcggcgagccgcagccattgccttttatggtaatcgtgcgagagggcgcag
ggacttcctttgtcccaaatctgtgcggagccgaaatctgggaggcgccgccgcaccccctctagcgggcgcggggcgaagcggtgcggc
gccggcaggaaggaaatgggcggggagggccttcgtgcgtcgccgcgccgccgtccccttctccctctccagcctcggggctgtccgcgg
ggggacggctgccttcgggggggacggggcagggcggggttcggcttctggcgtgtgaccggcggctctagagcctctgctaaccatgtt
catgccttcttctttttcctacagctcctgggcaacgtgctggttattgtgctgtctcatcattttggcaaagaattcctcgaagatcta
ggcaacgcgtctcgaggcggccgccgccaccatgcagtcaaaggtccttctcgcggtagctctgtggttgtgcgtagagacacgggccgc
ttcagtggggctgccttcagtaagcctcgatttgccccggctttcaatacagaaagatattctcacaatcaaggctaataccactctgca
aattacctgtcgcggccaaagggatctggattggctctggccgaataatcaatcagggagtgaacagcgggtcgaagtgaccgagtgttc
tgacggactatttgcaaaacacttactatcccaaaagtaatcgggaacgataccggggcttacaagtgtttctacagagagacagatctt
gctagtgtgatttacgtatatgtgcaagattaccgcagccctttcatagcatccgtatcagaccaacatggagttgtatacattacagag
aacaagaacaaaactgtagttattccctgtttggggtctataagcaacctcaacgtaagcctctgcgctagatacccagagaaacgattc
gtcccggatgggaatagaatttcctgggatagcaagaagggcttcactattccgagctacatgatatcatacgcaggtatggtgttctgc
gaagcgaagataaacgatgagagttatcagtcaatcatgtacatagtggtggtcgttgggtacagaatttatgatgttgtcctcagtccg
tcccatggcattgaactgagtgtcggggaaaaattggtgctgaattgtaccgctaggacagaattgaacgttggcattgactttaactgg
gagtatccgagctcaaagcaccagcataaaaagttggttaacagagatctgaaaacccagtctggcagtgaaatgaaaaagttcctctct
acgctgacaatagacggtgttacgaggtctgatcagggtttgtacacatgtgctgcatcttccggtttgatgacaaagaaaaactccacg
tttgttcgcgtgcatgagaagccgtttgtggcgttcggctcaggaatggagtccctcgttgaagcgacggtaggcgagcgggtccggatc
cccgctaaatacttgggctacccaccaccggagataaaatggtataagaatggtataccgcttgagagtaatcatacgataaaagcgggt
catgtactcaccataatggaagtaagcgagagagatactggaaattacaccgttatcctgacaaatccaatatccaaagagaagcagtca
cacgttgtatcactcgtggtttatgtcccccctcaaataggagaaaagtcactcatcagtccggtggatagttatcaatacgggacgacc
cagactctcacatgcacagtatatgccatccctccaccccaccatattcattggtattggcagcttgaagaggaatgcgccaatgagcct
tcacaggcagtgtcagttacaaatccttacccttgtgaggaatggaggagcgtcgaggacttccaggggggtaataagatagaagtcaat
aagaatcaatttgcactcatagaaggaaagaacaagacggtttctactttggtgatacaagccgcaaatgtgtccgcactctataagtgc
gaggcagtcaacaaagttggtcgcggcgagcgggtgataagcttccacgtcacgaggggaccggaaatcactcttcaacccgatatgcaa
cccacggaacaggaatctgtttcactttggtgtacggcggataggagtacgtttgagaacctcacatggtataaattgggaccgcagccc
ttgcctatccacgtgggggagcttcccactcctgtttgcaagaatctggatacactctggaaactgaatgctactatgttcagcaattct
accaatgacatactcattatggaattgaaaaatgcgtcactccaagatcagggagattacgtatgcctcgctcaggaccgcaagactaaa
aagaggcactgtgttgtccggcagcttacagttcttgaacgcgtggcgcctaccattaccggcaatctggagaatcaaactacgtcaatc
ggggagagcatcgaggtctcatgcacggccagcggcaatcctccgccccagattatgtggtttaaagacaatgagacgctggtggaggat
agtgggattgtacttaaggatgggaatcgcaacctcaccattcgaagagtgcgcaaggaggacgaagggttgtacacctgccaagcatgc
tccgttctgggatgcgcaaaagttgaagcattctttataaccagatctcgcaagaagcgcagcaccagatctcgcaagaagcgcagcggg
gcccccatgatatctctgatcgctgctcttgctgtggactacgtgattggtatggagaatgctatgccctggaatctgcctgcagacctg
gcatggtttaagaggaacacccttaataaacctgtaatcatgggacgacatacatgggagagtatcggcaggccattgcccgggaggaag
aatataattctgagttcccagccttctactgatgatagggtaacttgggtcaagagcgtcgacgaggccatcgccgcttgcggggatgtg
cctgaaatcatggttatcgggggcggacgggtgattgagcaatttctgcccaaggcacagaagctgtatctgacccatatagacgccgag
gtggagggagatacacactttcccgactatgagcccgatgattgggagagtgtctttagcgaatttcatgacgccgacgcacagaattcc
catagctactgcttcgaaattctcgaacggcgctaggcggccgcgcggatccagacatgataagatacattgatgagtttggacaaacca
caactagaatgcagtgaaaaaaatgctttatttgtgaaatttgtgatgctattgctttatttgtaaccattataagctgcaataaacaag
ttaacaacaacaattgcattcattttatgtttcaggttcagggggaggtgtgggaggttttttagtcgactggggagagatctgaggaac
ccctagtgatggagttggccactccctctctgcgcgctcgctcgctcactgaggccgcccgggcaaagcccgggcgtcgggcgacctttg
gtcgcccggcctcagtgagcgagcgagcgcgcagagagggagtggccaac
>VEGFR2 IgG 2-3-DD amino acid sequence
(SEQ ID NO: 31)
MKVLAAGVVPLLLVLHWKHGAGDYRSPFIASVSDQHGVVYITENKNKTVVIPCLGSISNLNVSLCARYPEKRFVPDGNRISWDSKKGFTI
PSYMISYAGMVFCEAKINDESYQSIMYIVVVVGYRIYDVVLSPSHGIELSVGEKLVLNCTARTELNVGIDFNWEYPSSKHQHKKLVNRDL
KTQSGSEMKKFLSTLTIDGVTRSDQGLYTCAASSGLMTKKNSTFVRVHEKPFVAFGSGMTRSRKKRSTRSRKKRSGAPMISLIAALAVDY
VIGMENAMPWNLPADLAWFKRNTLNKPVIMGRHTWESIGRPLPGRKNIILSSQPSTDDRVTWVKSVDEAIAACGDVPEIMVIGGGRVIEQ
FLPKAQKLYLTHIDAEVEGDTHFPDYEPDDWESVFSEFHDADAQNSHSYCFEILERR*
>rAAV vector encoding VEGFR2 IgG 2-3 (TMP-regulated IgG loops 2-3 of VEGFR2)
(SEQ ID NO: 32)
ttggccactccctctctgcgcgctcgctcgctcactgaggccgggcgaccaaaggtcgcccgacgcccgggctttgcccgggcggcctca
gtgagcgagcgagcgcgcagagagggagtggccaactccatcactaggggttcctcagatctgaattcggtacctagttattaatagtaa
tcaattacggggtcattagttcatagcccatatatggagttccgcgttacataacttacggtaaatggcccgcctggctgaccgcccaac
gacccccgcccattgacgtcaataatgacgtatgttcccatagtaacgccaatagggactttccattgacgtcaatgggtggagtattta
cggtaaactgcccacttggcagtacatcaagtgtatcatatgccaagtacgccccctattgacgtcaatgacggtaaatggcccgcctgg
cattatgcccagtacatgaccttatgggactttcctacttggcagtacatctacgtattagtcatcgctattaccatggtcgaggtgagc
cccacgttctgcttcactctccccatctcccccccctccccacccccaattttgtatttatttattttttaattattttgtgcagcgatg
ggggcggggggggggggggggcgcgcgccaggcggggcggggcggggcgaggggcggggcggggcgaggcggagaggtgcggcggcagcc
aatcagagcggcgcgctccgaaagtttccttttatggcgaggcggcggcggcggcggccctataaaaagcgaagcgcgcggcgggcggga
gtcgctgcgcgctgccttcgccccgtgccccgctccgccgccgcctcgcgccgcccgccccggctctgactgaccgcgttactcccacag
gtgagcgggcgggacggcccttctcctccgggctgtaattagcgcttggtttaatgacggcttgtttcttttctgtggctgcgtgaaagc
cttgaggggctccgggagggccctttgtgcggggggagcggctcggggggtgcgtgcgtgtgtgtgtgcgtggggagcgccgcgtgcggc
tccgcgctgcccggcggctgtgagcgctgcgggcgcggcgcggggctttgtgcgctccgcagtgtgcgcgaggggagcgcggccgggggc
ggtgccccgcggtgcggggggggctgcgaggggaacaaaggctgcgtgcggggtgtgtgcgtgggggggtgagcagggggtgtgggcgcg
tcggtcgggctgcaaccccccctgcacccccctccccgagttgctgagcacggcccggcttcgggtgcggggctccgtacggggcgtggc
gcggggctcgccgtgccgggcggggggtggcggcaggtgggggtgccgggcggggcggggccgcctcgggccggggagggctcgggggag
gggcgcggcggcccccggagcgccggcggctgtcgaggcgcggcgagccgcagccattgccttttatggtaatcgtgcgagagggcgcag
ggacttcctttgtcccaaatctgtgcggagccgaaatctgggaggcgccgccgcaccccctctagcgggcgcggggcgaagcggtgcggc
gccggcaggaaggaaatgggcggggagggccttcgtgcgtcgccgcgccgccgtccccttctccctctccagcctcggggctgtccgcgg
ggggacggctgccttcgggggggacggggcagggcggggttcggcttctggcgtgtgaccggcggctctagagcctctgctaaccatgtt
catgccttcttctttttcctacagctcctgggcaacgtgctggttattgtgctgtctcatcattttggcaaagaattcctcgaagatcta
ggcaacgcgtctcgaggcggccgccgccaccatgaaagtccttgccgccggagttgtccctctcttgcttgtactccactggaagcacgg
tgcaggagattatcgctcaccatttatagcgagcgtgtcagatcaacatggcgtggtctatataactgagaataagaataaaacggtcgt
tatcccctgtttgggtagcatatctaacctcaacgttagtttgtgcgctcgataccctgaaaagcggttcgtacccgatggcaatcggat
cagttgggacagtaaaaaaggcttcactattccgtcatacatgatcagctatgcagggatggttttctgcgaagctaaaatcaatgacga
gagttatcagagcattatgtacatcgtagttgtcgttgggtatcgaatttacgacgttgtgctgtccccttctcacggaattgaactctc
cgttggagaaaaactcgtattgaactgtacggcacgcacggaattgaatgtagggatagatttcaactgggagtatccatcatctaaaca
tcaacataaaaaacttgtaaatcgagacctgaagacacagagtgggtccgaaatgaaaaaatttcttagtactctgactatcgacggggt
aacaagatctgatcaagggctgtacacctgtgcggccagctcaggtcttatgacaaaaaaaaactccacttttgtcagagtgcatgaaaa
gccgttcgtggcatttggttcaggtatgaccagatctcgcaagaagcgcagcaccagatctcgcaagaagcgcagcggggcccccatgat
atctctgatcgctgctcttgctgtggactacgtgattggtatggagaatgctatgccctggaatctgcctgcagacctggcatggtttaa
gaggaacacccttaataaacctgtaatcatgggacgacatacatgggagagtatcggcaggccattgcccgggaggaagaatataattct
gagttcccagccttctactgatgatagggtaacttgggtcaagagcgtcgacgaggccatcgccgcttgcggggatgtgcctgaaatcat
ggttatcgggggcggacgggtgattgagcaatttctgcccaaggcacagaagctgtatctgacccatatagacgccgaggtggagggaga
tacacactttcccgactatgagcccgatgattgggagagtgtctttagcgaatttcatgacgccgacgcacagaattcccatagctactg
cttcgaaattctcgaacggcgctaggcggccgcgcggatccagacatgataagatacattgatgagtttggacaaaccacaactagaatg
cagtgaaaaaaatgctttatttgtgaaatttgtgatgctattgctttatttgtaaccattataagctgcaataaacaagttaacaacaac
aattgcattcattttatgtttcaggttcagggggaggtgtgggaggttttttagtcgactggggagagatctgaggaacccctagtgatg
gagttggccactccctctctgcgcgctcgctcgctcactgaggccgcccgggcaaagcccgggcgtcgggcgacctttggtcgcccggcc
tcagtgagcgagcgagcgcgcagagagggagtggccaac

1-36. (canceled)

37. A recombinant adeno-associated virus (rAAV) comprising:
(i) an isolated nucleic acid comprising an expression cassette flanked by adeno-associated virus (AAV) inverted terminal repeats (ITRs), wherein the expression cassette encodes a fusion protein comprising a therapeutic protein, a destabilization domain (DD), and an amino acid linker comprising one or more furin cleavage sites; and
(ii) one or more AAV capsid proteins.

38. The rAAV of claim 37, wherein the one or more AAV capsid proteins are AAV2, AAV3, AAV4, AAV5, AAV6, AAV7, AAV8, AAV9, or AAV10 capsid proteins.

39. The rAAV of claim 37, wherein the one or more AAV capsid proteins are AAV2, AAV2*, AAV6, or AAV6* capsid proteins.

40. The rAAV of claim 37, wherein the destabilization domain comprises a DHFR destabilization domain.

41. The rAAV of claim 37, wherein the AAV ITRs are AAV2 ITRs.

42. The rAAV of claim 37, wherein at least one of the AAV ITRs comprises a mutated terminal resolution site.

43. The rAAV of claim 37, wherein the therapeutic protein is a neurotrophic factor, transcription factor, growth factor, protective factor, a growth factor receptor subunit, or recombinase.

44. The rAAV of claim 43, wherein: the neurotrophic factor is a leukemia inhibitory factor (LIF), ciliary neurotrophic factor (CNTF), Endothelin 2 (EDN2), or Oncostatin M (OSM).

45. The rAAV of claim 43, wherein the transcription factor is Achaete-scute homolog 1 (ASCL1), Nuclear factor (erythroid-derived 2)-like 2 growth factor (NRF2), or Signal transducer and activator of transcription 3 (STAT3).

46. The rAAV of claim 43, wherein the growth factor is Heparin-binding EGF-like growth factor (HBEGF).

47. The rAAV of claim 43, wherein the protective factor is Retinal protective factor 2 (RPF2).

48. The rAAV of claim 43, wherein the growth factor receptor subunit is a vascular endothelial growth factor receptor 2 (VEGFR2) protein or a fragment thereof.

49. The rAAV of claim 43, wherein the recombinase is Cre recombinase.

50. The rAAV of claim 37, wherein the destabilization domain comprises an FK506 binding protein (FKBP) destabilization domain or a dihydrofolate reductase (DHFR) destabilization domain.

51. The rAAV of claim 50, wherein the DHFR destabilization domain binds to trimethoprim (TMP).

52. The rAAV of claim 50, wherein the DHFR destabilization domain is an E. coli destabilization domain.

53. The rAAV of claim 37, wherein the linker comprises 1, 2, or 3 furin cleavage sites.
